SEQUENCE LISTING (35 sequences)

SEQ ID NO: 1 - protein, 750 aa, Homo sapiens
MGKSESQMDITDINTPKPKKKQRWTPLEISLSVLVLLLTIIAVTMIALYATYDDGICKSSDCIKSAARLIQNMDATTEPCTDFFKYACGGWLKRNVIPETSSRYGNFDILRDELEVVLKDVLQEPKTEDIVAVQKAKALYRSCINESAIDSRGGEPLLKLLPDIYGWPVATENWEQKYGASWTAEKAIAQLNSKYGKKVLINLFVGTDDKNSVNHVIHIDQPRLGLPSRDYYECTGIYKEACTAYVDFMISVARLIRQEERLPIDENQLALEMNKVMELEKEIANATAKPEDRNDPMLLYNKMTLAQIQNNFSLEINGKPFSWLNFTNEIMSTVNISITNEEDVVVYAPEYLTKLKPILTKYSARDLQNLMSWRFIMDLVSSLSRTYKESRNAFRKALYGTTSETATWRRCANYVNGNMENAVGRLYVEAAFAGESKHVVEDLIAQIREVFIQTLDDLTWMDAETKKRAEEKALAIKERIGYPDDIVSNDNKLNNEYLELNYKEDEYFENIIQNLKFSQSKQLKKLREKVDKDEWISGAAVVNAFYSSGRNQIVFPAGILQPPFFSAQQSNSLNYGGIGMVIGHEITHGFDDNGRNFNKDGDLVDWWTQQSASNFKEQSQCMVYQYGNFSWDLAGGQHLNGINTLGENIADNGGLGQAYRAYQNYIKKNGEEKLLPGLDLNHKQLFFLNFAQVWCGTYRPEYAVNSIKTDVHSPGNFRIIGTLQNSAEFSEAFHCRKNSYMNPEKKCRVW

SEQ ID NO: 2 - protein, 699 aa, Homo sapiens
YDDGICKSSDCIKSAARLIQNMDATTEPCTDFFKYACGGWLKRNVIPETSSRYGNFDILRDELEVVLKDVLQEPKTEDIVAVQKAKALYRSCINESAIDSRGGEPLLKLLPDIYGWPVATENWEQKYGASWTAEKAIAQLNSKYGKKVLINLFVGTDDKNSVNHVIHIDQPRLGLPSRDYYECTGIYKEACTAYVDFMISVARLIRQEERLPIDENQLALEMNKVMELEKEIANATAKPEDRNDPMLLYNKMTLAQIQNNFSLEINGKPFSWLNFTNEIMSTVNISITNEEDVVVYAPEYLTKLKPILTKYSARDLQNLMSWRFIMDLVSSLSRTYKESRNAFRKALYGTTSETATWRRCANYVNGNMENAVGRLYVEAAFAGESKHVVEDLIAQIREVFIQTLDDLTWMDAETKKRAEEKALAIKERIGYPDDIVSNDNKLNNEYLELNYKEDEYFENIIQNLKFSQSKQLKKLREKVDKDEWISGAAVVNAFYSSGRNQIVFPAGILQPPFFSAQQSNSLNYGGIGMVIGHEITHGFDDNGRNFNKDGDLVDWWTQQSASNFKEQSQCMVYQYGNFSWDLAGGQHLNGINTLGENIADNGGLGQAYRAYQNYIKKNGEEKLLPGLDLNHKQLFFLNFAQVWCGTYRPEYAVNSIKTDVHSPGNFRIIGTLQNSAEFSEAFHCRKNSYMNPEKKCRVW

SEQ ID NO: 3 - protein, 116 aa, Artificial
  humanized variable heavy
EVQLQESGGGVVQPGGSLRLSCAASGFTFSNYGMHWIRQAPGKGLEWVSYISSGSSTIYYADSVKGRFTISRDNSKNTLYLQMNSLRSEDTAVYYCARRGLLLDYWGQGTLVTVSS

SEQ ID NO: 4 - protein, 111 aa, Artificial
  humanized variable light chain
DIQMTQSPSSLSASVGDRVTISCRASKSVSTSSYSYMHWYQQKPEKAPKLLIKYASYLQSGVPSRFSGSGSGTDFTLTISSLQPEDVATYYCQHSREFPWTFGAGTKLELK

SEQ ID NO: 5 - protein, 13 aa, Artificial
  glycine-serine linker
GGSGGGSGGGSGG

SEQ ID NO: 6 - protein, 224 aa, Artificial
  humanized heavy chain Fd
EVQLQESGGGVVQPGGSLRLSCAASGFTFSNYGMHWIRQAPGKGLEWVSYISSGSSTIYYADSVKGRFTISRDNSKNTLYLQMNSLRSEDTAVYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 7 - protein, 218 aa, Artificial
  humanized light chain
DIQMTQSPSSLSASVGDRVTISCRASKSVSTSSYSYMHWYQQKPEKAPKLLIKYASYLQSGVPSRFSGSGSGTDFTLTISSLQPEDVATYYCQHSREFPWTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 108 aa, Artificial
  heavy chain IgG1 CH1 and truncated hinge constant domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 9 - protein, 107 aa, Artificial [sequence omitted]

SEQ ID NO: 10 - protein, 8 aa, Artificial
  heavy chain variable (VH) domain CDR1
GFTFSNYG

SEQ ID NO: 11 - protein, 8 aa, Artificial
  heavy chain variable (VH) domain CDR2
ISSGSSTI

SEQ ID NO: 12 - protein, 9 aa, Artificial
  heavy chain variable (VH) domain CDR3
ARRGLLLDY

SEQ ID NO: 13 - protein, 10 aa, Artificial
  light chain variable (VL) domain CDR1
KSVSTSSYSY

SEQ ID NO: 14 - protein, 3 aa, Artificial
  light chain variable (VL) domain CDR2
YAS

SEQ ID NO: 15 - protein, 9 aa, Artificial
  light chain variable (VL) domain CDR3
QHSREFPWT

SEQ ID NO: 16 - protein, 15 aa, Artificial
  GS3 linker
GGGGSGGGGSGGGGS

SEQ ID NO: 17 - protein, 116 aa, Artificial
  murine 3E10 heavy chain variable domain (VH)
EVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSS

SEQ ID NO: 18 - protein, 111 aa, Artificial
  murine 3E10 light chain variable domain (VL)
DIVLTQSPASLAVSLGQRATISCRASKSVSTSSYSYMHWYQQKPGQPPKLLIKYASYLESGVPARFSGSGSGTDFHLNIHPVEEEDAATYYCQHSREFPWTFGGGTKLELK

SEQ ID NO: 19 - protein, 5 aa, Artificial
  heavy chain variable domain CDR1 of murine 3E10 VH
NYGMH

SEQ ID NO: 20 - protein, 17 aa, Artificial
  heavy chain variable domain CDR2 of murine 3E10 VH
YISSGSSTIYYADTVKG

SEQ ID NO: 21 - protein, 7 aa, Artificial
  heavy chain variable domain CDR3 of murine 3E10 VH
RGLLLDY

SEQ ID NO: 22 - protein, 15 aa, Artificial
  light chain variable domain CDR1 of murine 3E10 VL
RASKSVSTSSYSYMH

SEQ ID NO: 23 - protein, 7 aa, Artificial
  light chain variable domain CDR2 of murine 3E10 VL
YASYLES

SEQ ID NO: 24 - protein, 9 aa, Artificial
  light chain variable domain CDR3 of murine 3E10 VL
QHSREFPWT

SEQ ID NO: 25 - protein, 17 aa, Artificial
  heavy chain variable domain CDR2
YISSGSSTIYYADSVKG

SEQ ID NO: 26 - protein, 15 aa, Artificial
  light chain variable domain CDR1
RASKSVSTSSYSYLA

SEQ ID NO: 27 - protein, 7 aa, Artificial
  light chain variable domain CDR2
YASYLQS

SEQ ID NO: 28 - protein, 242 aa, Artificial
  humanized Fv3E10
DIQMTQSPSSLSASVGDRVTISCRASKSVSTSSYSYMHWYQQKPEKAPKLLIKYASYLQSGVPSRFSGSGSGTDFTLTISSLQPEDVATYYCQHSREFPWTFGAGTKLELKGGGGSGGGGSGGGGSEVQLQESGGGVVQPGGSLRLSCAASGFTFSNYGMHWIRQAPGKGLEWVSYISSGSSTIYYADSVKGRFTISRDNSKNTLYLQMNSLRSEDTAVYYCARRGLLLDYWGQGTLVTVSS

SEQ ID NO: 29 - protein, 936 aa, Artificial
  heavy chain + neprilysin ectodomain polypeptide
EVQLQESGGGVVQPGGSLRLSCAASGFTFSNYGMHWIRQAPGKGLEWVSYISSGSSTIYYADSVKGRFTISRDNSKNTLYLQMNSLRSEDTAVYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGSGGGSGGGSGGYDDGICKSSDCIKSAARLIQNMDATTEPCTDFFKYACGGWLKRNVIPETSSRYGNFDILRDELEVVLKDVLQEPKTEDIVAVQKAKALYRSCINESAIDSRGGEPLLKLLPDIYGWPVATENWEQKYGASWTAEKAIAQLNSKYGKKVLINLFVGTDDKNSVNHVIHIDQPRLGLPSRDYYECTGIYKEACTAYVDFMISVARLIRQEERLPIDENQLALEMNKVMELEKEIANATAKPEDRNDPMLLYNKMTLAQIQNNFSLEINGKPFSWLNFTNEIMSTVNISITNEEDVVVYAPEYLTKLKPILTKYSARDLQNLMSWRFIMDLVSSLSRTYKESRNAFRKALYGTTSETATWRRCANYVNGNMENAVGRLYVEAAFAGESKHVVEDLIAQIREVFIQTLDDLTWMDAETKKRAEEKALAIKERIGYPDDIVSNDNKLNNEYLELNYKEDEYFENIIQNLKFSQSKQLKKLREKVDKDEWISGAAVVNAFYSSGRNQIVFPAGILQPPFFSAQQSNSLNYGGIGMVIGHEITHGFDDNGRNFNKDGDLVDWWTQQSASNFKEQSQCMVYQYGNFSWDLAGGQHLNGINTLGENIADNGGLGQAYRAYQNYIKKNGEEKLLPGLDLNHKQLFFLNFAQVWCGTYRPEYAVNSIKTDVHSPGNFRIIGTLQNSAEFSEAFHCRKNSYMNPEKKCRVW

SEQ ID NO: 30 - protein, 954 aa, Artificial
  scFv-neprilysin
DIQMTQSPSSLSASVGDRVTISCRASKSVSTSSYSYMHWYQQKPEKAPKLLIKYASYLQSGVPSRFSGSGSGTDFTLTISSLQPEDVATYYCQHSREFPWTFGAGTKLELKGGGGSGGGGSGGGGSEVQLQESGGGVVQPGGSLRLSCAASGFTFSNYGMHWIRQAPGKGLEWVSYISSGSSTIYYADSVKGRFTISRDNSKNTLYLQMNSLRSEDTAVYYCARRGLLLDYWGQGTLVTVSSGGSGGGSGGGSGGYDDGICKSSDCIKSAARLIQNMDATTEPCTDFFKYACGGWLKRNVIPETSSRYGNFDILRDELEVVLKDVLQEPKTEDIVAVQKAKALYRSCINESAIDSRGGEPLLKLLPDIYGWPVATENWEQKYGASWTAEKAIAQLNSKYGKKVLINLFVGTDDKNSVNHVIHIDQPRLGLPSRDYYECTGIYKEACTAYVDFMISVARLIRQEERLPIDENQLALEMNKVMELEKEIANATAKPEDRNDPMLLYNKMTLAQIQNNFSLEINGKPFSWLNFTNEIMSTVNISITNEEDVVVYAPEYLTKLKPILTKYSARDLQNLMSWRFIMDLVSSLSRTYKESRNAFRKALYGTTSETATWRRCANYVNGNMENAVGRLYVEAAFAGESKHVVEDLIAQIREVFIQTLDDLTWMDAETKKRAEEKALAIKERIGYPDDIVSNDNKLNNEYLELNYKEDEYFENIIQNLKFSQSKQLKKLREKVDKDEWISGAAVVNAFYSSGRNQIVFPAGILQPPFFSAQQSNSLNYGGIGMVIGHEITHGFDDNGRNFNKDGDLVDWWTQQSASNFKEQSQCMVYQYGNFSWDLAGGQHLNGINTLGENIADNGGLGQAYRAYQNYIKKNGEEKLLPGLDLNHKQLFFLNFAQVWCGTYRPEYAVNSIKTDVHSPGNFRIIGTLQNSAEFSEAFHCRKNSYMNPEKKCRVW

SEQ ID NO: 31 - protein, 699 aa, Artificial
  G399V/G714K human neprilysin variant
YDDGICKSSDCIKSAARLIQNMDATTEPCTDFFKYACGGWLKRNVIPETSSRYGNFDILRDELEVVLKDVLQEPKTEDIVAVQKAKALYRSCINESAIDSRGGEPLLKLLPDIYGWPVATENWEQKYGASWTAEKAIAQLNSKYGKKVLINLFVGTDDKNSVNHVIHIDQPRLGLPSRDYYECTGIYKEACTAYVDFMISVARLIRQEERLPIDENQLALEMNKVMELEKEIANATAKPEDRNDPMLLYNKMTLAQIQNNFSLEINGKPFSWLNFTNEIMSTVNISITNEEDVVVYAPEYLTKLKPILTKYSARDLQNLMSWRFIMDLVSSLSRTYKESRNAFRKALYVTTSETATWRRCANYVNGNMENAVGRLYVEAAFAGESKHVVEDLIAQIREVFIQTLDDLTWMDAETKKRAEEKALAIKERIGYPDDIVSNDNKLNNEYLELNYKEDEYFENIIQNLKFSQSKQLKKLREKVDKDEWISGAAVVNAFYSSGRNQIVFPAGILQPPFFSAQQSNSLNYGGIGMVIGHEITHGFDDNGRNFNKDGDLVDWWTQQSASNFKEQSQCMVYQYGNFSWDLAGGQHLNGINTLGENIADNGGLGQAYRAYQNYIKKNGEEKLLPGLDLNHKQLFFLNFAQVWCGTYRPEYAVNSIKTDVHSPKNFRIIGTLQNSAEFSEAFHCRKNSYMNPEKKCRVW

SEQ ID NO: 32 - protein, 19 aa, Artificial
  Native human immunoglobulin signal sequence (VH3-49)
MEFGLSWLFLVAILKGVQC

SEQ ID NO: 33 - protein, 955 aa, Artificial
  H3 Fab-linker-Neprilysin heavy chain
MEFGLSWLFLVAILKGVQCEVQLQESGGGVVQPGGSLRLSCAASGFTFSNYGMHWIRQAPGKGLEWVSYISSGSSTIYYADSVKGRFTISRDNSKNTLYLQMNSLRSEDTAVYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGSGGGSGGGSGGYDDGICKSSDCIKSAARLIQNMDATTEPCTDFFKYACGGWLKRNVIPETSSRYGNFDILRDELEVVLKDVLQEPKTEDIVAVQKAKALYRSCINESAIDSRGGEPLLKLLPDIYGWPVATENWEQKYGASWTAEKAIAQLNSKYGKKVLINLFVGTDDKNSVNHVIHIDQPRLGLPSRDYYECTGIYKEACTAYVDFMISVARLIRQEERLPIDENQLALEMNKVMELEKEIANATAKPEDRNDPMLLYNKMTLAQIQNNFSLEINGKPFSWLNFTNEIMSTVNISITNEEDVVVYAPEYLTKLKPILTKYSARDLQNLMSWRFIMDLVSSLSRTYKESRNAFRKALYGTTSETATWRRCANYVNGNMENAVGRLYVEAAFAGESKHVVEDLIAQIREVFIQTLDDLTWMDAETKKRAEEKALAIKERIGYPDDIVSNDNKLNNEYLELNYKEDEYFENIIQNLKFSQSKQLKKLREKVDKDEWISGAAVVNAFYSSGRNQIVFPAGILQPPFFSAQQSNSLNYGGIGMVIGHEITHGFDDNGRNFNKDGDLVDWWTQQSASNFKEQSQCMVYQYGNFSWDLAGGQHLNGINTLGENIADNGGLGQAYRAYQNYIKKNGEEKLLPGLDLNHKQLFFLNFAQVWCGTYRPEYAVNSIKTDVHSPGNFRIIGTLQNSAEFSEAFHCRKNSYMNPEKKCRVW

SEQ ID NO: 34 - protein, 22 aa, Artificial
  Native human immunoglobulin signal sequence
MDMRVPAQLLGLLLLWLRGARC

SEQ ID NO: 35 - protein, 240 aa, Artificial
  L2 light chain
MDMRVPAQLLGLLLLWLRGARCDIQMTQSPSSLSASVGDRVTISCRASKSVSTSSYSYMHWYQQKPEKAPKLLIKYASYLQSGVPSRFSGSGSGTDFTLTISSLQPEDVATYYCQHSREFPWTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2021/0395716 A1
(43) Pub. Date: Dec. 23, 2021

(54) METHODS AND COMPOSITIONS FOR TREATMENT OF PROTEIN AGGREGATION-BASED DISORDERS

(71) Applicant: Valerion Therapeutics, LLC, Concord, MA (US)

(72) Inventor: Dustin D. Armstrong, Quincy, MA (US)

(21) Appl. No.: 17/284,768

(22) PCT Filed: Oct. 11, 2019
(86) PCT No.: PCT/US19/55982

§ 371 Date: Apr. 12, 2021

Related U.S. Application Data
(60) Provisional application No. 62/744,613, filed on Oct. 11, 2018.
(60) Provisional application No. 62/760,914, filed on Nov. 13, 2018.
(60) Provisional application No. 62/760,911, filed on Nov. 13, 2018.

(51) Int. Cl. C12N 9/64; C12N 15/67

(52) U.S. Cl. CPC C12N 9/6494; C12Y 304/24011; C12N 15/67

(57) ABSTRACT

In certain embodiments, the present disclosure provides compositions and methods for treating protein aggregation-based disorders (e.g., Age-related Macular Degeneration (AMD), Inclusion Body Myositis (IBM), Myofibrillar Myopathy 2 (MFM2), and Nemaline Myopathy (NM)).

### RELATED APPLICATION(S)

This application claims benefit to U.S. Provisional Application No. 62/744,613, filed on Oct. 11, 2018, U.S. Provisional Application No. 62/760,911, filed on Nov. 13, 2018, and U.S. Provisional Application No. 62/760,914, filed on Nov. 13, 2018, the contents of which are hereby incorporated by reference in its entirety.

### BACKGROUND OF THE DISCLOSURE

A variety of human diseases arise as a result of offending protein aggregation or inclusion body (IB) formation, from aging disorders such as Age-Related Macular Degeneration (AMD) to rare genetic disorders such as Desminopathies (e.g., MFM2). IBs can impede normal protein function by removing functional proteins from their active location, and they can trigger cytotoxic protein interactions. Thus, clearance of extracellular plaques and intracellular IBs is an attractive therapeutic objective toward preventing disease progression, and may allow the body's natural clearance mechanisms to catch up for a return to a pre-disease state. However, there are currently no treatments for AMD or MFM2 that clear drusen or muscle protein aggregates.

### SUMMARY OF THE DISCLOSURE

There is a need in the art for methods and compositions for degrading offending protein aggregates or inclusion bodies in patients with protein aggregation-based disorders, as well as a need for alternative therapies for treating protein aggregation-based disorders. Such methods and compositions would improve treatment of protein aggregation-based disorders. The present disclosure provides such methods and compositions. In certain embodiments, the methods and compositions provided herein decrease protein aggregates or inclusion bodies. Similarly, the methods and compositions provided herein can be used to improve symptoms of protein aggregation-based disorders (e.g., Age-related Macular Degeneration, Inclusion Body Myositis, Myofibrillar Myopathy 2, or Nemaline Myopathy).

In some embodiments, the disclosure provides a chimeric polypeptide comprising: (i) a neprilysin polypeptide, and (ii) an internalizing moiety.

In some aspects, the neprilysin polypeptide is a functional neprilysin polypeptide, comprises a functional portion of a neprilysin polypeptide, or comprises a functional fragment of a neprilysin polypeptide. In some aspects, the neprilysin polypeptide does not comprise the full-length neprilysin polypeptide set forth in SEQ ID NO: 1. In some aspects, neither the neprilysin polypeptide nor the chimeric polypeptide comprises the portion of neprilysin polypeptide set forth in residues 1-51 of SEQ ID NO: 1. In some aspects, the neprilysin polypeptide comprises the portion of neprilysin polypeptide set forth in residues 52-750 of SEQ ID NO: 1. In certain aspects, the neprilysin polypeptide comprises the amino acid sequence of SEQ ID NO: 2. In some aspects, the chimeric polypeptide has neprilysin activity.

In some aspects, the internalizing moiety comprises an antibody or antigen binding fragment. The antibody or antigen binding fragment may be a chimeric, humanized, or fully human antibody or antigen binding fragment. In some aspects, the antibody or antigen binding fragment comprises a heavy chain variable domain and a light chain variable domain. The heavy chain variable (VH) domain may comprise the amino acid sequence of SEQ ID NO: 3, and in some aspects, the VH domain comprises: a VH CDR1 having the amino acid sequence of SEQ ID NO: 10; a VH CDR2 having the amino acid sequence of SEQ ID NO: 11; a VH CDR3 having the amino acid sequence of SEQ ID NO: 12, which CDRs are according to the IMGT system. The light chain variable (VL) domain may comprise the amino acid sequence of SEQ ID NO: 4, and in some aspects, the VL domain comprises: a VL CDR1 having the amino acid sequence of SEQ ID NO: 13; a VL CDR2 having the amino acid sequence of SEQ ID NO: 14; a VL CDR3 having the amino acid sequence of SEQ ID NO: 15, which CDRs are according to the IMGT system.

In some aspects, the internalizing moiety comprises an antibody (e.g., a monoclonal antibody). In some aspects, the internalizing moiety comprises an antigen-binding fragment (e.g., a Fab, Fab′, or an scFv).

In some aspects, the internalizing moiety promotes delivery of the chimeric polypeptide into cells via an equilibrative nucleoside transporter (ENT) transporter (e.g., via ENT2). In some aspects, the internalizing moiety promotes delivery of the chimeric polypeptide into a muscle cell (e.g., skeletal muscle cell), a retinal cell, and/or a neuronal cell.

In some aspects, the neprilysin polypeptide is chemically conjugated to the internalizing moiety. The chimeric polypeptide may comprise a fusion protein comprising the neprilysin polypeptide and all or a portion of the internalizing moiety. In some aspects, the chimeric polypeptide does not include a linker interconnecting the neprilysin polypeptide to the internalizing moiety. In some aspects, the fusion protein comprises a linker, and the linker may conjugate or join the neprilysin polypeptide to the internalizing moiety. In some aspects, the linker is a cleavable linker. In some aspects, all or a portion of the internalizing moiety is conjugated or joined, directly or via a linker, to the N-terminal amino acid of the neprilysin polypeptide or to the C-terminal amino acid of the neprilysin polypeptide. In some aspects, all or a portion of the internalizing moiety is conjugated or joined, directly or indirectly to an internal amino acid of the neprilysin polypeptide.

In some aspects, the chimeric polypeptide is capable of degrading protein aggregates (e.g., in a cell). In some aspects, the chimeric polypeptide is capable of degrading protein aggregates in a cell from a subject having Age-related Macular Degeneration (AMD), Inclusion Body Myositis (IBM), Myofibrillar Myopathy 2 (MFM2), Nemaline Myopathy (NM), or Alzheimer's Disease. The subject may be a non-human animal (e.g., a mouse) or the subject may be a human. In some aspects, the cell is in vitro. In some aspects, cell is a muscle cell (e.g., a skeletal muscle cell), a neuronal cell, or a retinal cell.

In some aspects, the chimeric polypeptide is produced recombinantly. The chimeric polypeptide may be produced in a prokaryotic or eukaryotic cell. In some aspects, the eukaryotic cell is selected from a yeast cell, an avian cell, an insect cell, or a mammalian cell.

In some embodiments, the disclosure provides a nucleic acid construct, comprising a nucleotide sequence that encodes the chimeric polypeptide as described herein, as a chimeric polypeptide comprising a fusion protein. The nucleotide sequence may be codon optimized for expression in a mammalian cell (e.g., a CHO cell or a HEK-293 cell).

In some embodiments, the disclosure provides a set of nucleic acid constructs, together comprising nucleotide sequences that encode the chimeric polypeptide as described herein. The nucleotide sequences may be codon optimized for expression in a mammalian cell (e.g., a CHO cell or a HEK-293 cell).

In some embodiments, the disclosure provides vector comprising the nucleic acid construct as described herein. In some embodiments, the disclosure provides a set of vectors comprising the set of nucleic acid constructs as described herein. In some embodiments, the disclosure provides a host cell comprising a vector as described herein.

In some embodiments, the disclosure provides methods for delivering neprilysin activity into a cell from or of a subject having Age-related Macular Degeneration (AMD), comprising contacting the cell with a chimeric polypeptide described herein.

In some embodiments, the disclosure provides methods for delivering neprilysin activity into a cell from or of a subject having Inclusion Body Myositis (IBM), comprising contacting the cell with a chimeric polypeptide described herein.

In some embodiments, the disclosure provides methods for delivering neprilysin activity into a cell from or of a subject having Myofibrillar Myopathy 2 (MFM2), comprising contacting the cell with a chimeric polypeptide described herein.

In some embodiments, the disclosure provides methods for delivering neprilysin activity into a cell from or of a subject having Nemaline Myopathy (NM), comprising contacting the cell with a chimeric polypeptide as described herein.

In some aspects, the subject is a non-human animal (e.g., a mouse) or is a human. In some aspects, the cell is in the subject. The cell may be a muscle cell (e.g., a skeletal muscle cell), a brain cell, or a retinal cell. In some aspects, the cell is in vitro.

In some embodiments, the disclosure provides methods for treating a subject having Age-related Macular Degeneration (AMD), comprising administering to the subject a therapeutically effective amount of a chimeric polypeptide described herein.

In some embodiments, the disclosure provides methods for treating a subject having Inclusion Body Myositis (IBM), comprising administering to the subject a therapeutically effective amount of a chimeric polypeptide described herein.

In some embodiments, the disclosure provides methods for treating a subject having Myofibrillar Myopathy 2 (MFM2), comprising administering to the subject a therapeutically effective amount of a chimeric polypeptide described herein.

In some embodiments, the disclosure provides methods for treating a subject having Nemaline Myopathy (NM), comprising administering to the subject a therapeutically effective amount of a chimeric polypeptide described herein.

In some aspects, the subject is a non-human animal (e.g., is a mouse) or is a human.

The disclosure contemplates all combinations of any of the foregoing aspects and embodiments, as well as combinations with any of the embodiments set forth in the detailed description and examples. For example, any of the chimeric polypeptides and compositions, including chimeric polypeptides and compositions having any combination of neprilysin portions and an internalizing moiety portions, can be used in any of the methods described herein.

### BRIEF DESCRIPTION OF THE FIGURES

The patent or application file contains at least one drawing executed in color. Copies of this patent or patent application publication with color drawings will be provided by the Office upon request and payment of the necessary fee.

FIG. 1 provides a Fab-NEP heavy chain (NEP ectodomain: residues 52-750 from GenBank accession number NP_001341571) and hu3E10 kappa light chain. Components of the Fab-NEP heavy chain include: hu3E10 VH(green)-huIgG1 heavy chain CH1 domain (Kabat EU residues 118-215)-truncated huIgG1 heavy chain hinge domain (Kabat EU residues 216-225)(orange)-13-residue flexible GlySer linker(grey)-human NEP ectodomain (yellow) (residues 52-750 from Genbank accession number NP_00134157). IMGT CDRs are shown in green within the VH domain. For the variant G399V/G714K human NEPv ectodomain described by Webster et al, 2014, PLoS One 9: e104001, substitute G399V/G714K at the Gly residues highlighted in bold. G399V/G714K neprilysin numbering is based upon SEQ ID: 2 in US 2012/0237496. Components of the light chain include: hu3E10 VL-human kappa light chain constant domain (blue). IMGT CDRs are shown in blue within the VL domain.

FIG. 2 provides a hu3E10 scFv-NEP fusion protein. The design of the fusion protein is based upon the Mu3E10scFv-MTM fusion protein (VL-linker-VH-linker-MTM1) described in Lawlor et al, 2013, Hum Mol Genet 22: 1525. The components of the fusion protein include: hu3E10 VL (blue)−3X(Gly4Ser) linker (red)—hu3E10 VH (green)−13-residue flexible GlySer linker (grey)—human NEP ectodomain (yellow) (residues 52-750 from Genbank accession number NP_00134157). IMGT light chain CDRs are shown in blue within the VL domain and IMGT heavy chain CDRs are shown in green within the VH domain.

FIG. 3 provides a chart summarizing activity per μl for various neprilysin constructs, including GH-NEP (WT) (control growth hormone fused to neprilysin, which is secreted into the media); Fab-NEP (bAmyl) (Fab-neprilysin fusion, where the neprilysin is the G399V/G714K variant); Fab-NEP (WT) (Fab-neprilysin fusion, where the neprilysin is wild type neprilysin ectodomain); and hu3E10 (Fab alone). The activity assay was performed using a synthetic substrate. Concentrations were not quantitated and normalized, but activity was identified for all of the fusions. Fab-NEP (WT) appears to demonstrate better activity than the Fab-NEP (bAmyl), but the titer of the variant Fab-NEP fusion was significantly lower than that of the wild type Fab-NEP.

FIGS. 4A-4D provide SDS-PAGE of hu3E10 and various neprilysin fusion protein constructs. HEK293 cells were grown in 10-cm plate with DMEM supplemented with 5% FBS. During transfection, serum free medium was used and PEI was used for transfection. PEI to DNA ratio is 3:1. Ten microgram plasmid DNA was applied. However, for the hu3E10 light chain and Fab-NEP heavy chain co-transfection, a total of 15 Ig were used with 1:1 ratio. After 4 hr transfection, medium containing PEI/DNA was removed and replaced with fresh serum free medium. After 3 days incubation at 37° C., media were collected and clarified by centrifugation. (The pH of the conditioned media dropped to 6-6.5, and 35 μl 2M Tris was added to bring back to neutral pH) based on the color of phenol red). Clarified media were boiled in 4×SDS sample buffer in the presence and absence of b-ME. Forty microliter were examined on gel. The expression reports demonstrate that Fab-NEP expresses better than the Fab-NEP mutant (FIGS. 4A-4C). In addition, roughly equivalent expression of the fusion protein (Fab-NEP) was confirmed at different transfection ratios, as well as secretion of the Fab-NEP into the media (FIG. 4D).

FIG. 5 provides a diagram depicting a neprilysin polypeptide (A), a Fab-NEP (B), and a sc-Fv-NEP (C).

FIG. 6 provides an image demonstrating clinical presentation of AMD.

FIG. 7 provides an example staining of ApoE (green), GPNMB (red, abundant RPE membrane protein), and DAPI (blue). See Johnson et al. PNAS (2011) 108(45):18277-18282.

FIGS. 8A-8B provide images of the structure and active site of neprilysin. Neprilysin is an integral membrane type-II zinc-dependent endopeptidase (750 amino acids, predicted molecular weight 85.5 kDa). See Bayes-Genis, et al., J Am Coll Cardiol. (2016) 68(6):639-653.

FIGS. 9A-9B demonstrate the mechanism of AMD. FIG. 9A provides a diagram of the structure of a normal eye and retina (left panel) and an expanded view of the cell types and structures in a segment of the retina within the macula (right panel). FIG. 9B provides a diagram of retinas with dry AMD. The retina showing early/intermediate AMD is provided in the left panel and a retina having geographic atrophy is shown in the right panel. See van Lookeren Campagne et al. J. Pathol (2014) 232:151-164.

FIG. 10 provides images demonstrating Inclusion Body Myositis with basophilic rimmed vacuoles (left panel) and vacuole filled with granules (right panel). See Dr. Lohit Chauhan. LinkedIn SlideShare Aug. 31, 2016 (slideshare.net/lohitchauhan/inflammatory-myopathies).

FIG. 11 provides diagrams demonstrating the difference between normal muscle (top panel) and muscle effected by Inclusion Body Myositis (bottom panel). See Myositis Support and Understanding (understandingmyositis.org/myositis/inclusion-body-myositis)

FIG. 12 provides Gomori trichrome stain images of muscle fibers effected by Myofibrillar Myopathy 2. See Neuromuscular: Myofibrillar (Desmin) Myopathy (neuromuscular.wustl.edu/pathol/desmin.htm).

FIG. 13 provides a schematic of the normal state of skeletal muscle and demonstrates the interaction of Desmin with various organelles (e.g., mitochondria). See Neuromuscular: Muscular Dystrophy Syndromes (neuromuscular.wustl.edu/musdist/lg.html#desmin).

FIG. 14 provides a schematic of the normal state of skeletal muscle and depicts the relative location and binding partners of proteins of interest (e.g., CryAB and Desmin). See Capetanaki et a. Exp Cell Res. (2007) 313(10):2063-2076.

FIG. 15 provides an image showing Gomori trichrome staining of muscle-biopsy sections. Gomori trichrome treatment results in deep-purple staining of Nemaline bodies (indicated by arrows), which were observed in muscle-biopsy sections from patients having severe congenital Nemaline Myopathy. See Ilkovski et al., Am. J. Hum. Genet. (2001) 68:1333-1343.

FIG. 16 provides electron microscopy images of muscle samples from patients having severe congenital Nemaline Myopathy. The top panel shows intranuclear rods (short arrow) and buildup of glycogen (long arrow). The bottom panel shows dramatically atrophied muscle fibers. See Ilkovski et al., Am. J. Hum. Genet. (2001) 68:1333-1343.

FIG. 17 provides expression verification using SDS-PAGE. Forty microliters of CCS was evaluated via reducing (top panels) and non-reducing (bottom panels) SDS-PAGE on 4-20% Tris-glycine TGX gels (Bio-Rad), stained with Instant Blue gel stain (left panels) or western blotted to PVDF (right panels). The western blots were incubated with rabbit anti-Neprilysin 1° antibody (Millipore Sigma #AB5458) at a 1:1000 dilution and goat anti-Rabbit IgG-AP 2° antibody (Bio-Rad #170-6518) at a 1:1000 dilution. Immunoreactivity was detected using an alkaline phosphatase conjugate substrate kit (Bio-Rad). Lanes from left to right of each panel: (1) Molecular weight markers; (2) 293-6E negative control CCS; (3) Fab-NEP 293-6E CCS.

FIG. 18 demonstrates purification of Fab-NEP. Left panel shows affinity chromatography fraction analysis by non-reducing SDS-PAGE. Lanes from left to right: (1) molecular weight markers (10 μL); (2) column load (20 μL); (3) non-bound (20 μL); (4-6) pH 4.0 elution fractions 4-6 (5 μL each); (7-9) pH 3.6 elution fractions 2-4 (5 μL); (10) resin slurry (5 μL). Right panel affinity chromatography fraction analysis by non-reducing SDS-PAGE of pooled Fab-NEP protein. Lanes from left to right: (I) column load (20 μL); (2) nonbound (20 μL); (3) pH 4.0 elution pool (4 μg); (4) pH 3.6 elution pool (4 μg); (5) molecular-weight markers (10 μL). 4-20% Tris-glycine TGX gels were stained with Instant Blue gel stain (top panel and bottom left panel) or western blotted to PVDF (bottom right panel). The western blot was incubated with rabbit anti-Neprilysin 1° antibody (Millipore Sigma #AB5458) at a 1:1000 dilution and goat anti-Rabbit IgG-AP 2° antibody (Bio-Rad #170-6518) at a 1:1000 dilution. Immunoreactivity was detected using an alkaline phosphatase conjugate substrate kit (Bio-Rad).

FIG. 19 demonstrates purification of Fab-NEP. A 4-20% Tris-glycine TGX gel, reducing and non-reducing SDS-PAGE, stained with Instant Blue stain is provided. Lanes left to right: (1) molecular weight markers (10 μL); (2) pH 4.0 elution pool (+BME) (4 μg); (3) blank; (4) pH 4.0 elution pool (−BME) (4 μg); (5) blank; (6) molecular weight markers (10 μL); (7) pH 3.6 elution pool (+BME); (8) blank; (9) pH 3.6 elution pool (−BME).

FIG. 20 provides an analytical size exclusion chromatography of Fab-NEP. The chromatographs provide a freeze/thaw analysis. Analytical SEC chromatographs with final samples in blue (upper and middle panels), final samples after freeze/thaw in red (upper and middle panels; nearly indistinguishable from final sample) and molecular weight standards in blue (lower panel).

FIG. 21 shows Fab-NEP degrades β-amyloid peptide (1-42). The Fab conjugate does not appear to inhibit neprilysin activity, as compared to yeast NEP (yNEP).

FIG. 22 shows Fab-NEP penetrates cells. Fab-NEP uptake by T47D cells is measured by flow cytometry via fluorescein labeling. Error bars indicate range of n+2. Fab-NEP is internalized by cells after 24 hr.

FIGS. 23A-23F demonstrate Fab-NEP prevents accumulation of β-amyloid(1-42) fused to a GFP reporter in C2C12 cells. FIGS. 23A-23F provide images at 10×. Cells were washed and fixed in PFA after 24 hours incubation with Texas Red-labeled Fab-NEP. FIG. 23A shows pre-treatment with 100 μg/mL Fab-NEP, 24 hours before transfection with 500 ng GFP reporter plasmid. FIG. 23B provides same as FIG. 23A except 0 μg/nL Fab-NEP. FIG. 23C shows left vertical axis, columns: normalized GFP signal for 24 hour post-, 4 hour post-, and pre-transfection treatment with Fab-NEP. Right vertical axis, lines: normalized signal from Texas Red-labeled Fab-NEP. FIGS. 23D-23F provide 40× images of cells treated 24 hours post-transfection to note β-amyloid(1-42) pattern (FIG. 23D), Fab-NEP (FIG. 23E), and overlay with DAPI (FIG. 23F). Empty arrows point to co-localized Fab-NEP and GFP; shaded arrow points to a cell not exposed to Fab-NEP. Images are representative and correspond to nuclei (blue), β-amyloid(1-42) fused to GFP (green), and Fab-NEP (red).

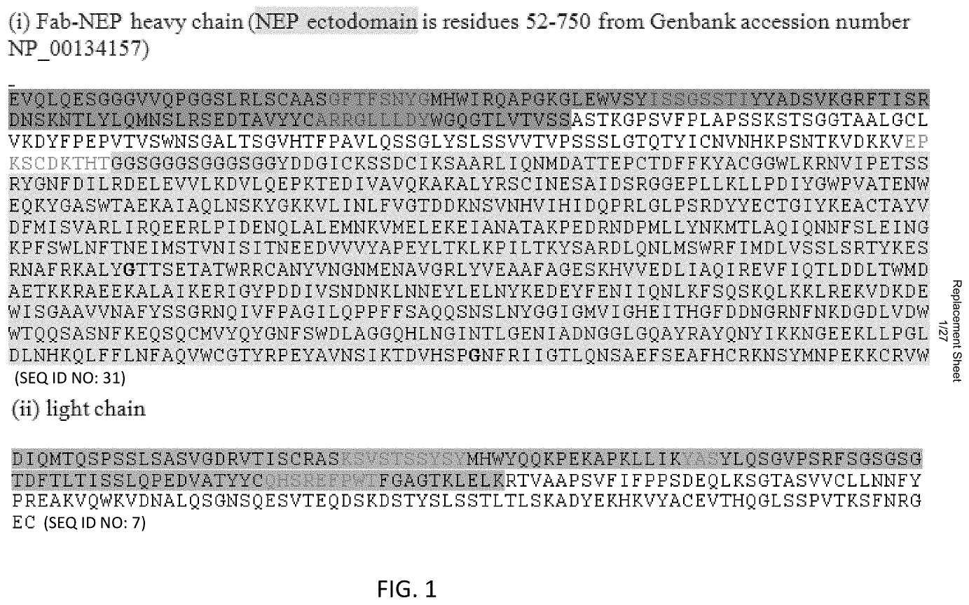

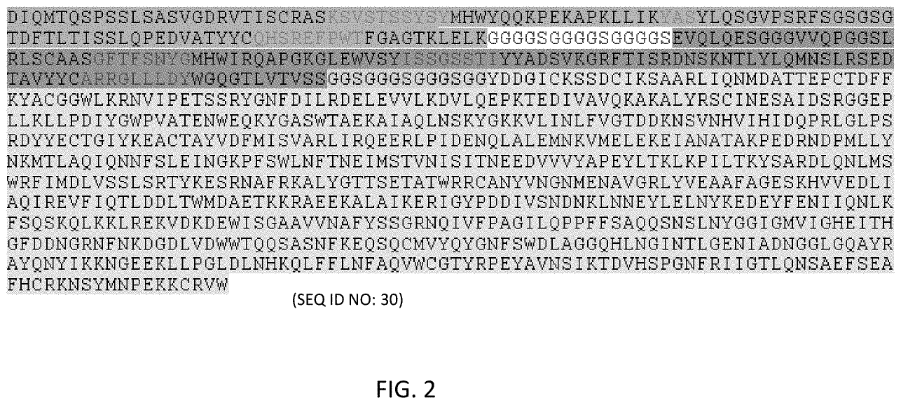

### DETAILED DESCRIPTION OF THE DISCLOSURE

Neprilysin is a ubiquitously-expressed zinc-dependent endopeptidase that has been shown to degrade a variety of substrates, including β-amyloid. In certain embodiments, the disclosure provides a chimeric polypeptide comprising a neprilysin polypeptide and an internalizing moiety, as described herein. The chimeric polypeptide has the potential to be a novel treatment approach to a variety of diseases arising as a result of offending protein aggregation or inclusion body formation, from aging disorders, such as Age-Related Macular Degeneration (AMD), to rare genetic disorders, such as Desminopathies (e.g., MFM2).

In some embodiments, the disclosure provides a chimeric polypeptide comprising a neprilysin polypeptide and an internalizing moiety, as described herein. Any such chimeric polypeptide of the disclosure can comprise any of the neprilysin polypeptides described herein associated with any of the internalizing moiety portions described herein, and these chimeric polypeptides can be used in any of the methods of the disclosure.

Thus, in certain aspects, the disclosure provides chimeric polypeptides comprising a mature neprilysin polypeptide (e.g., a neprilysin ectodomain polypeptide) that may be used to treat symptoms associated with protein aggregation-based disorders.

In certain embodiments, the disclosure provides a chimeric polypeptide comprising (i) a neprilysin polypeptide; and (ii) an internalizing moiety that promotes delivery into cells, such as into cytoplasm of cells. In a particular embodiment, the internalizing moiety helps target delivery of the chimeric polypeptide to muscle cells, retinal cells, and neuronal cells.

### I. Neprilysin Polypeptides

In certain embodiments, the non-internalizing moiety polypeptide portion of a chimeric polypeptide of the disclosure (or a chimeric polypeptide for use in the methods of the disclosure) is a neprilysin polypeptide. In other words, in certain embodiments, neprilysin-containing chimeric polypeptides are provided. Exemplary neprilysin (e.g., a mature neprilysin or neprilysin ectodomain) polypeptides for use in the methods and compositions of the disclosure are provided herein. Neprilysin is a ubiquitously-expressed zinc-dependent endopeptidase that has been shown to target small (<3000 Da) peptides and to degrade a variety of substrates, including β-amyloid. (Bayes-Genis, et al., J Am Coll Cardiol. (2016) 68(6):639-653). It is a 750 amino acid protein that is 85.5 kDa with 1 transmembrane helix (GenBank accession no. NP_001341571). The soluble version (e.g., amino acids 52-750 of SEQ ID NO: 1) is fully active. In some aspects, it is referred to as a common acute lymphoblastic leukemia antigen (CALLA). Neprilysin is likely monomeric as there are currently no known direct binding partners. In some aspects, neprilysin is constitutively expressed in many organs, and is developmentally regulated in some cell types (lymphocytes and granulocytes) (D'Adamio et al. J. Immunol. (1989) 142, 283-287). In some aspects, neprilysin may lower inflammation response (Bayes-Genis, et al., J Am Coll Cardiol. (2016) 68(6):639-653). In some embodiments, the neprilysin (e.g., a mature neprilysin) polypeptides have utility in clearing beta amyloid in diseased cells. In some embodiments, the diseased cells are from a subject having Age-related Macular Degeneration (AMD), Inclusion Body Myositis (IBM), Myofibrilla Myopathy 2 (MFM2), and/or Nemaline Myopathy (NM). In some embodiments, the diseased cells are from a subject having Age-related Macular Degeneration. In other embodiments, the diseased cells are from a subject having Myofibrilla Myopathy 2.

As used herein, the neprilysin polypeptides include various functional fragments and variants, fusion proteins, and modified forms of the wildtype neprilysin polypeptide. In particular embodiments, the neprilysin is a mature neprilysin (e.g., is a neprilysin ectodomain). In some embodiments, the neprilysin polypeptide lacks the short intracellular domain and transmembrane helix (amino acids 1-51 of SEQ ID NO: 1). Exemplary mature neprilysin polypeptides include polypeptides having residues 52-750 of SEQ ID NO: 1. Such variants or isoforms, functional fragments or variants, fusion proteins, and modified forms of the neprilysin polypeptides have at least a portion of the amino acid sequence of substantial sequence identity to the native neprilysin polypeptide, and retain the function of the native neprilysin polypeptide. It should be noted that “retain the function” does not mean that the activity of a particular fragment must be identical or substantially identical to that of the native protein although, in some embodiments, it may be. However, to retain the native activity, that native activity should be at least 50%, at least 60%, at least 70%, at least 75%, at least 80%, at least 85%, at least 90%, at least 95% that of the native protein to which such activity is being compared, with the comparison being made under the same or similar conditions. In some embodiments, retaining the native activity may include scenarios in which a fragment or variant has improved activity versus the native protein to which such activity is being compared, e.g., at least 105%, at least 110%, at least 120%, or at least 125%, with the comparison being bade under the same or similar conditions.

In certain embodiments, a functional fragment, variant, or fusion protein of a neprilysin polypeptide comprises an amino acid sequence that is at least 80%, 85%, 90%, 95%, 97%, 98%, 99% or 100% identical to a neprilysin polypeptide, such as a neprilysin ectodomain polypeptide (e.g., at least 80%, 85%, 90%, 95%, 97%, 98%, 99% or 100% identical to SEQ ID NO: 2), or fragments thereof.

Here and elsewhere in the specification, sequence identity refers to the percentage of residues in the candidate sequence that are identical with the residue of a corresponding sequence to which it is compared, after aligning the sequences and introducing gaps, if necessary to achieve the maximum percent identity for the entire sequence, and not considering any conservative substitutions as part of the sequence identity. Neither N- or C-terminal extensions nor insertions shall be construed as reducing identity or homology.

Methods and computer programs for the alignment of sequences and the calculation of percent identity are well known in the art and readily available. Sequence identity may be measured using sequence analysis software. For example, alignment and analysis tools available through the ExPasy bioinformatics resource portal, such as ClustalW algorithm, set to default parameters. Suitable sequence alignments and comparisons based on pair-wise or global alignment can be readily selected. One example of an algorithm that is suitable for determining percent sequence identity and sequence similarity is the BLAST algorithm, which is described in Altschul et al., J Mol Biol 215:403-410 (1990). Software for performing BLAST analyses is publicly available through the National Center for Biotechnology Information (ncbi.nlm.nih.gov/). In certain embodiments, the now current default settings for a particular program are used for aligning sequences and calculating percent identity.

In certain embodiments, the neprilysin polypeptide for use in the chimeric polypeptides and methods of the disclosure is a full-length or substantially full-length neprilysin polypeptide, or a mature form of a full-length neprilysin. In certain embodiments, the neprilysin polypeptide for use in the chimeric polypeptide and methods of the disclosure is a functional fragment that has neprilysin activity.

In certain embodiments of any of the foregoing, the neprilysin portion of the chimeric polypeptide of the disclosure comprises a neprilysin polypeptide (e.g., a mature form), which in certain embodiments may be a functional fragment of a neprilysin polypeptide or may be a substantially full-length neprilysin polypeptide.

In some embodiments, the neprilysin is the mature form of a neprilysin (e.g., neprilysin ectodomain). In particular embodiments, the mature form of the neprilysin corresponds to amino acids 52-750 of SEQ ID NO: 1 (GenBank accession number NP_001341571). A neprilysin polypeptide corresponding to SEQ ID NO: 2 is a soluble neprilysin and is active. (Bayes-Genis et al., J Am Coll Cardiol. (2016) 68(6):639-653). In some embodiments, the mature form of the neprilysin corresponds to an amino acid sequence that is at least 80%, 85%, 90%, 91%, 92%, 93%, 94%, 95%, 96%, 97%, 98%, 99% or 100% identical to the amino acid sequence of SEQ ID NO: 2, or functional fragments thereof.

Suitable neprilysin polypeptides or functional fragments thereof for use in the chimeric polypeptides and methods of the disclosure have neprilysin activity, as evaluated in vitro or in vivo. Exemplary functional fragments comprise, at least 100, at least 150, at least 200, at least 250, at least 300, at least 325, at least 350, at least 375, at least 400, at least 425, at least 450, at least 475, at least 500, at least 525, at least 550, at least 575, at least 600, at least 625, at least 650, at least 675, or at least 690 consecutive amino acid residues of a full-length neprilysin polypeptide (e.g., SEQ ID NO: 1). In some embodiments, the functional fragment comprises 100-200, 100-300, 100-400, 100-500, 100-550, 100-600, 100-650, 200-690, 300-690, 400-690, 500-690, 600-690 consecutive amino acids of a mature neprilysin polypeptide (e.g., SEQ ID NO: 2). Similarly, in certain embodiments, the disclosure contemplates chimeric proteins where the neprilysin portion is a variant of any of the foregoing neprilysin polypeptides or bioactive fragments. Exemplary variants have an amino acid sequence at least 90%, 92%, 95%, 96%, 97%, 98%, or at least 99% identical to the amino acid sequence of a native (e.g. mature) neprilysin polypeptide or functional fragment thereof, and such variants retain the neprilysin variant's activity. The disclosure contemplates chimeric polypeptides and the use of such polypeptides wherein the neprilysin portion comprises any of the neprilysin polypeptides, fragments, or variants described herein in combination with any internalizing moiety described herein. Moreover, in certain embodiments, the neprilysin portion of any of the foregoing chimeric polypeptides may, in certain embodiments, be a fusion protein. Any such chimeric polypeptides comprising any combination of neprilysin portions and internalizing moiety portions, and optionally including one or more linkers, one or more tags, etc., may be used in any of the methods of the disclosure.

In other embodiments, the neprilysin polypeptides may be glycosylated, or may be not glycosylated. In some embodiments, the neprilysin polypeptide is glycosylated at N145 and/or N628 of SEQ ID NO: 1. In some aspects, glycosylation is necessary for surface expression and endopeptidase activity of the neprilysin polypeptide.

In certain embodiments, mature neprilysin, or fragments or variants are human mature neprilysin.

In certain embodiments, fragments or variants of the neprilysin polypeptides (e.g., neprilysin ectodomain polypeptides) can be obtained by screening polypeptides recombinantly produced from the corresponding fragment of the nucleic acid encoding a neprilysin polypeptide. In addition, fragments or variants can be chemically synthesized using techniques known in the art such as conventional Merrifield solid phase f-Moc or t-Boc chemistry. The fragments or variants can be produced (recombinantly or by chemical synthesis) and tested to identify those fragments or variants that can function as a native neprilysin protein, for example, by testing their ability to degrade offending protein aggregates and/or treat symptoms of protein aggregation-based disorders.

In certain embodiments, the present disclosure contemplates modifying the structure of a neprilysin polypeptide for such purposes as enhancing therapeutic or prophylactic efficacy, or stability (e.g., ex vivo shelf life and resistance to proteolytic degradation in vivo). Such modified neprilysin polypeptides are considered functional equivalents of the naturally-occurring neprilysin polypeptide. Modified polypeptides can be produced, for instance, by amino acid substitution, deletion, or addition. For instance, it is reasonable to expect, for example, that an isolated replacement of a leucine with an isoleucine or valine, an aspartate with a glutamate, a threonine with a serine, or a similar replacement of an amino acid with a structurally related amino acid (e.g., conservative mutations) will not have a major effect on the neprilysin biological activity of the resulting molecule. Conservative replacements are those that take place within a family of amino acids that are related in their side chains.

This disclosure further contemplates generating sets of combinatorial mutants of a neprilysin polypeptide, as well as truncation mutants, and is especially useful for identifying functional variant sequences. Combinatorially-derived variants can be generated which have a selective potency relative to a naturally occurring neprilysin polypeptide. Likewise, mutagenesis can give rise to variants which have intracellular half-lives dramatically different than the corresponding wild-type neprilysin polypeptide. For example, the altered protein can be rendered either more stable or less stable to proteolytic degradation or other cellular process which result in destruction of, or otherwise inactivation of neprilysin function. Such variants can be utilized to alter the neprilysin polypeptide level by modulating their half-life. There are many ways by which the library of potential neprilysin variants sequences can be generated, for example, from a degenerate oligonucleotide sequence. Chemical synthesis of a degenerate gene sequence can be carried out in an automatic DNA synthesizer, and the synthetic genes then be ligated into an appropriate gene for expression. The purpose of a degenerate set of genes is to provide, in one mixture, all of the sequences encoding the desired set of potential polypeptide sequences. The synthesis of degenerate oligonucleotides is well known in the art (see for example, Narang, SA (1983) Tetrahedron 39:3; Itakura et al., (1981) Recombinant DNA, Proc. 3rd Cleveland Sympos. Macromolecules, ed. AG Walton, Amsterdam: Elsevier pp 273-289; Itakura et al., (1984) Annu. Rev. Biochem. 53:323; Itakura et al., (1984) Science 198:1056; Ike et al., (1983) Nucleic Acid Res. 11:477). Such techniques have been employed in the directed evolution of other proteins (see, for example, Scott et al., (1990) Science 249:386-390; Roberts et al., (1992) PNAS USA 89:2429-2433; Devlin et al., (1990) Science 249: 404-406; Cwirla et al., (1990) PNAS USA 87: 6378-6382; as well as U.S. Pat. Nos. 5,223,409, 5,198,346, and 5,096,815).

Alternatively, other forms of mutagenesis can be utilized to generate a combinatorial library. For example, neprilysin polypeptide variants can be generated and isolated from a library by screening using, for example, alanine scanning mutagenesis and the like (Ruf et al., (1994) Biochemistry 33:1565-1572; Wang et al., (1994) J. Biol. Chem. 269:3095-3099; Balint et al., (1993) Gene 137:109-118; Grodberg et al., (1993) Eur. J. Biochem. 218:597-601; Nagashima et al., (1993) J. Biol. Chem. 268:2888-2892; Lowman et al., (1991) Biochemistry 30:10832-10838; and Cunningham et al., (1989) Science 244:1081-1085), by linker scanning mutagenesis (Gustin et al., (1993) Virology 193:653-660; Brown et al., (1992) Mol. Cell Biol. 12:2644-2652; McKnight et al., (1982) Science 232:316); by saturation mutagenesis (Meyers et al., (1986) Science 232:613); by PCR mutagenesis (Leung et al., (1989) Method Cell Mol Biol 1:11-19); or by random mutagenesis, including chemical mutagenesis, etc. (Miller et al., (1992) A Short Course in Bacterial Genetics, CSHL Press, Cold Spring Harbor, N.Y.; and Greener et al., (1994) Strategies in Mol Biol 7:32-34). Linker scanning mutagenesis, particularly in a combinatorial setting, is an attractive method for identifying truncated (bioactive) forms of the neprilysin polypeptide.

A wide range of techniques are known in the art for screening gene products of combinatorial libraries made by point mutations and truncations, and, for that matter, for screening cDNA libraries for gene products having a certain property. Such techniques will be generally adaptable for rapid screening of the gene libraries generated by the combinatorial mutagenesis of the neprilysin polypeptides. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Each of the illustrative assays described below are amenable to high through-put analysis as necessary to screen large numbers of degenerate sequences created by combinatorial mutagenesis techniques.

In certain embodiments, a neprilysin polypeptide may include a peptidomimetic. As used herein, the term “peptidomimetic” includes chemically modified peptides and peptide-like molecules that contain non-naturally occurring amino acids, peptoids, and the like. Peptidomimetics provide various advantages over a peptide, including enhanced stability when administered to a subject. Methods for identifying a peptidomimetic are well known in the art and include the screening of databases that contain libraries of potential peptidomimetics. For example, the Cambridge Structural Database contains a collection of greater than 300,000 compounds that have known crystal structures (Allen et al., Acta Crystallogr. Section B, 35:2331 (1979)). Where no crystal structure of a target molecule is available, a structure can be generated using, for example, the program CONCORD (Rusinko et al., J. Chem. Inf. Comput. Sci. 29:251 (1989)). Another database, the Available Chemicals Directory (Molecular Design Limited, Informations Systems; San Leandro Calif.), contains about 100,000 compounds that are commercially available and also can be searched to identify potential peptidomimetics of the neprilysin polypeptides.

In certain embodiments, a neprilysin polypeptide may further comprise post-translational modifications. Exemplary post-translational protein modification include phosphorylation, acetylation, methylation, ADP-ribosylation, ubiquitination, glycosylation, carbonylation, sumoylation, biotinylation or addition of a polypeptide side chain or of a hydrophobic group. As a result, the modified neprilysin polypeptides may contain non-amino acid elements, such as lipids, poly- or mono-saccharide, and phosphates. Effects of such non-amino acid elements on the functionality of a neprilysin polypeptide may be tested for its biological activity. In certain embodiments, the neprilysin polypeptide may further comprise one or more polypeptide portions that enhance one or more of in vivo stability, in vivo half life, uptake/administration, and/or purification. In other embodiments, the internalizing moiety comprises an antibody or an antigen-binding fragment thereof.

In some embodiments, a neprilysin polypeptide is modified to increase β-amyloid degradation activity. In some aspects, the neprilysin ectodomain polypeptide comprises a double glycine modification to optimize the neprilysin for β-amyloid (SEQ ID NO: 31). Such a variant is described in US 2012/0237496 and Webster et al. PLOS ONE (2014) 9(8):e104001, both of which are incorporated by reference, and is identified as neprilysin variant G399V/G714K.

In one specific embodiment of the present disclosure, a neprilysin polypeptide may be modified with nonproteinaceous polymers. In one specific embodiment, the polymer is polyethylene glycol (“PEG”), polypropylene glycol, or polyoxyalkylenes, in the manner as set forth in U.S. Pat. Nos. 4,640,835; 4,496,689; 4,301,144; 4,670,417; 4,791,192 or 4,179,337. PEG is a well-known, water soluble polymer that is commercially available or can be prepared by ring-opening polymerization of ethylene glycol according to methods well known in the art (Sandler and Karo, Polymer Synthesis, Academic Press, New York, Vol. 3, pages 138-161).

By the terms “biological activity”, “bioactivity” or “functional” is meant the ability of the neprilysin polypeptide to carry out the functions associated with wildtype neprilysin polypeptide, for example, the degradation of a variety of substrates, including β-amyloid, as well as the degradation of offending protein aggregates. The terms “biological activity”, “bioactivity”, and “functional” are used interchangeably herein. As used herein, “fragments” are understood to include bioactive fragments (also referred to as functional fragments) or bioactive variants that exhibit “bioactivity” as described herein. That is, bioactive fragments or variants of neprilysin exhibit bioactivity that can be measured and tested. For example, bioactive fragments/functional fragments or variants exhibit the same or substantially the same bioactivity as native (i.e., wild-type, or normal) neprilysin polypeptide, and such bioactivity can be assessed by the ability of the fragment or variant to, e.g., degrade offending protein aggregates. As used herein, “substantially the same” refers to any parameter (e.g., activity) that is at least 70% of a control against which the parameter is measured. In certain embodiments, “substantially the same” also refers to any parameter (e.g., activity) that is at least 75%, 80%, 85%, 90%, 92%, 95%, 97%, 98%, 99%, 100%, 102%, 105%, or 110% of a control against which the parameter is measured, when assessed under the same or substantially the same conditions. In certain embodiments, fragments or variants of the neprilysin polypeptide will preferably retain at least 50%, 60%, 70%, 80%, 85%, 90%, 95% or 100% of the neprilysin biological activity associated with the native neprilysin polypeptide, when assessed under the same or substantially the same conditions.

In certain embodiments, fragments or variants of the neprilysin polypeptide have a half-life (t_{1/2}) which is enhanced relative to the half-life of the native protein. Preferably, the half-life of neprilysin fragments or variants is enhanced by at least 10%, 20%, 30%, 40%, 50%, 60%, 70%, 80%, 90%, 100%, 125%, 150%, 175%, 200%, 250%, 300%, 400% or 500%, or even by 1000% relative to the half-life of the native neprilysin polypeptide, when assessed under the same or substantially the same conditions. In some embodiments, the protein half-life is determined in vitro, such as in a buffered saline solution or in serum. In other embodiments, the protein half-life is an in vivo half-life, such as the half-life of the protein in the serum or other bodily fluid of an animal. In addition, fragments or variants can be chemically synthesized using techniques known in the art such as conventional Merrifield solid phase f-Moc or t-Boc chemistry. The fragments or variants can be produced (recombinantly or by chemical synthesis) and tested to identify those fragments or variants that can function as well as or substantially similarly to a native neprilysin polypeptide.

With respect to methods of increasing neprilysin bioactivity in cells, the disclosure contemplates all combinations of any of the foregoing aspects and embodiments, as well as combinations with any of the embodiments set forth in the detailed description and examples. The described methods based on administering chimeric polypeptides or contacting cells with chimeric polypeptides can be performed in vitro (e.g., in cells or culture) or in vivo (e.g., in a patient or animal model). In certain embodiments, the method is an in vitro method. In certain embodiments, the method is an in vivo method.

In some aspects, the present disclosure also provides a method of producing any of the foregoing chimeric polypeptides as described herein. Further, the present disclosure contemplates any number of combinations of the foregoing methods and compositions.

In certain aspects, a neprilysin polypeptide may be a fusion protein which further comprises one or more fusion domains. Well-known examples of such fusion domains include, but are not limited to, polyhistidine, Glu-Glu, glutathione S transferase (GST), thioredoxin, protein A, protein G, and an immunoglobulin heavy chain constant region (Fc), maltose binding protein (MBP), which are particularly useful for isolation of the fusion proteins by affinity chromatography. For the purpose of affinity purification, relevant matrices for affinity chromatography, such as glutathione-, amylase-, and nickel- or cobalt-conjugated resins are used. Fusion domains also include “epitope tags,” which are usually short peptide sequences for which a specific antibody is available. Well known epitope tags for which specific monoclonal antibodies are readily available include FLAG, influenza virus haemagglutinin (HA), His, and c-myc tags. It is recognized that any such tags or fusions may be appended to the neprilysin portion of the chimeric polypeptide or may be appended to the internalizing moiety portion of the chimeric polypeptide, or both.

In some cases, the fusion domains have a protease cleavage site, such as for Factor Xa or Thrombin, which allows the relevant protease to partially digest the fusion proteins and thereby liberate the recombinant proteins therefrom. The liberated proteins can then be isolated from the fusion domain by subsequent chromatographic separation. In certain embodiments, the neprilysin polypeptides may contain one or more modifications that are capable of stabilizing the polypeptides. For example, such modifications enhance the in vitro half-life of the polypeptides, enhance circulatory half-life of the polypeptides or reducing proteolytic degradation of the polypeptides.

In some embodiments, a neprilysin polypeptide may be a fusion protein with an Fc region of an immunoglobulin. As is known, each immunoglobulin heavy chain constant region comprises four or five domains. The domains are named sequentially as follows: CH1-hinge-CH2-CH3(-CH4). The DNA sequences of the heavy chain domains have cross-homology among the immunoglobulin classes, e.g., the CH2 domain of IgG is homologous to the CH2 domain of IgA and IgD, and to the CH3 domain of IgM and IgE. As used herein, the term, “immunoglobulin Fc region” is understood to mean the carboxyl-terminal portion of an immunoglobulin chain constant region, preferably an immunoglobulin heavy chain constant region, or a portion thereof. For example, an immunoglobulin Fc region may comprise 1) a CH1 domain, a CH2 domain, and a CH3 domain, 2) a CH1 domain and a CH2 domain, 3) a CH1 domain and a CH3 domain, 4) a CH2 domain and a CH3 domain, or 5) a combination of two or more domains and an immunoglobulin hinge region. In a preferred embodiment the immunoglobulin Fc region comprises at least an immunoglobulin hinge region a CH2 domain and a CH3 domain, and preferably lacks the CH1 domain. In one embodiment, the class of immunoglobulin from which the heavy chain constant region is derived is IgG (Igγ) (γ subclasses 1, 2, 3, or 4). Other classes of immunoglobulin, IgA (Igα), IgD (Igδ), IgE (IgE) and IgM (Igμ), may be used. The choice of appropriate immunoglobulin heavy chain constant regions is discussed in detail in U.S. Pat. Nos. 5,541,087, and 5,726,044. The choice of particular immunoglobulin heavy chain constant region sequences from certain immunoglobulin classes and subclasses to achieve a particular result is considered to be within the level of skill in the art. The portion of the DNA construct encoding the immunoglobulin Fc region preferably comprises at least a portion of a hinge domain, and preferably at least a portion of a CH₃ domain of Fc γ or the homologous domains in any of IgA, IgD, IgE, or IgM. Furthermore, it is contemplated that substitution or deletion of amino acids within the immunoglobulin heavy chain constant regions may be useful in the practice of the disclosure. One example would be to introduce amino acid substitutions in the upper CH2 region to create an Fc variant with reduced affinity for Fc receptors (Cole et al. (1997) J. Immunol. 159:3613). One of ordinary skill in the art can prepare such constructs using well known molecular biology techniques.

In certain embodiments of any of the foregoing, the neprilysin portion of the chimeric polypeptide comprises a neprilysin polypeptide, which in certain embodiments may be a functional fragment of a neprilysin polypeptide. In some embodiments, the neprilysin portion of the chimeric polypeptide comprises a mature form of neprilysin, e.g., a neprilysin ectodomain. In certain embodiments, such neprilysin ectodomain polypeptide or functional fragment thereof retains the ability to degrade offending protein aggregates. Further, in certain embodiments, the chimeric polypeptide that comprises such a neprilysin ectodomain polypeptide or functional fragment thereof can degrade offending protein aggregates. Exemplary bioactive fragments comprise at least 100, at least 150, at least 200, at least 250, at least 300, at least 325, at least 350, at least 375, at least 400, at least 425, at least 450, at least 475, at least 500, at least 525, at least 550, at least 575, at least 600, at least 625, at least 650, at least 675, or at least 690 consecutive amino acid residues of a full-length neprilysin ectodomain polypeptide (SEQ ID NO: 2).

In certain embodiments, the neprilysin polypeptide portion of the chimeric proteins described herein comprise a mature form of neprilysin (e.g., a neprilysin ectodomain polypeptide) but does not comprise a neprilysin polypeptide set forth in SEQ ID NO: 1. In some embodiments, the neprilysin polypeptide lacks at least a portion of the short intracellular domain and transmembrane helix corresponding to amino acids 1-51 of SEQ ID NO: 1. In some embodiments, the neprilysin polypeptide does not comprise a contiguous amino acid sequence corresponding to the amino acids 1-45, 1-46, 1-47, 1-48, 1-49, 1-50, or 1-51 of SEQ ID NO: 1. In particular embodiments, the neprilysin polypeptide does not comprise a contiguous amino acid sequence corresponding to the amino acids 1-51 of SEQ ID NO: 1.

In certain embodiments, the neprilysin polypeptide portion of the chimeric proteins described herein comprise a variant form of neprilysin (e.g., a neprilysin variant ectodomain polypeptide) as set forth in SEQ ID NO: 31.

In certain embodiments, the disclosure contemplates chimeric proteins where the neprilysin portion is a variant of any of the foregoing neprilysin polypeptides or functional fragments. Exemplary variants have an amino acid sequence at least 90%, 92%, 95%, 96%, 97%, 98%, or at least 99% identical to the amino acid sequence of a native neprilysin polypeptide or functional fragment thereof, and such variants retain the activity of native neprilysin. The disclosure contemplates chimeric polypeptides and the use of such polypeptides wherein the neprilysin portion comprises any of the neprilysin polypeptides, forms, or variants described herein in combination with any internalizing moiety described herein. An exemplary neprilysin polypeptide (e.g., neprilysin ectodomain polypeptides) is set forth in SEQ ID NO: 2. Moreover, in certain embodiments, the neprilysin portion of any of the foregoing chimeric polypeptides may, in certain embodiments, be a fusion protein. Any such chimeric polypeptides comprising any combination of neprilysin portions and internalizing moiety portions, and optionally including one or more linkers, one or more tags, etc., may be used in any of the methods of the disclosure.

### II. Internalizing Moieties

As used herein, the term “internalizing moiety” refers to a polypeptide/protein capable of interacting with a target tissue or a cell type such that the moiety is internalized into the target tissue or the cell type.

As used herein, “antibodies or antigen binding fragments of the disclosure” refer to any one or more of the antibodies and antigen binding fragments provided herein. Antibodies and antigen binding fragments of the disclosure comprise a heavy chain comprising a heavy chain variable domain and a light chain comprising a light chain variable domain. A V_{H} domain comprises three CDRs, such as any of the CDRs provided herein and as defined or identified by the Kabat and/or IMGT systems. These CDRs are typically interspersed with framework regions (FR), and together comprise the V_{H} domain. Similarly, a VL comprises three CDRs, such as any of the CDRs provided herein and as defined by the Kabat and/or IMGT systems. These CDRs are typically interspersed with framework regions (FR), and together comprise the V_{L} domain. The FR regions, such as FR1, FR2, FR3, and/or FR4 can similarly be defined or identified by the Kabat or IMGT systems. Throughout the application, when CDRs are indicated as being identified or defined by the Kabat or IMGT systems, what is meant is that the CDRs are in accordance with that system (e.g., the Kabat CDRs or the IMGT CDRs). Any of these terms can be used to indicate whether the Kabat or IMGT CDRs are being referred to.

The disclosure contemplates that an antibody or antigen binding fragment may comprise any combination of a V_{H} domain, as provided herein, and a V_{L} domain, as provided herein. In certain embodiments, at least one of the V_{H} and/or V_{L} domains are humanized (collectively, antibodies or antigen binding fragments of the disclosure). Chimeric antibodies are also included. Any antibody or antigen binding fragment of the disclosure may be provided alone. In other embodiments, any antibody or antigen binding fragment of the disclosure may be provided as a conjugate associated with a heterologous agent. Non-limiting examples of heterologous agents, which may include polypeptides, peptides, small molecules (e.g., a chemotherapeutic agent small molecule), or polynucleotides, are provided herein. Conjugates may refer to an antibody or antigen binding fragment associated with a heterologous agent.

In some embodiments, the antibody or antigen-binding fragment is isolated and/or purified. Any of the antibodies or antigen-binding fragments described herein, including those provided in an isolated or purified form, may be provided as a composition, such as a composition comprising an antibody or antigen-binding fragment formulated with one or more pharmaceutical and/or physiological acceptable carriers and/or excipients. Any of the antibodies or antigen-binding fragments described herein, including compositions (e.g., pharmaceutical compositions) may be used in any of the methods described herein and may be optionally provided conjugated (e.g., interconnected; associated) with a heterologous agent. In some embodiments, the internalizing moiety is capable of interacting with a target tissue or a cell type to effect delivery of the heterologous agent into a cell (i.e., penetrate desired cell; transport across a cellular membrane; deliver across cellular membranes to, at least, the cytoplasm). Such conjugates may similarly be provided as a composition and may be used in any of the methods described herein.

Internalizing moieties having limited cross-reactivity are generally preferred. In certain embodiments, this disclosure relates to an internalizing moiety which selectively, although not necessarily exclusively, targets and penetrates muscle, neuronal, and/or retinal cells. In certain embodiments, the internalizing moiety has limited cross-reactivity, and thus preferentially targets a particular cell or tissue type. However, it should be understood that internalizing moieties of the subject disclosure do not exclusively target specific cell types. Rather, the internalizing moieties promote delivery to one or more particular cell types, preferentially over other cell types, and thus provide for delivery that is not ubiquitous. In certain embodiments, suitable internalizing moieties include, for example, antibodies, monoclonal antibodies, or derivatives or analogs thereof. In certain embodiments, the internalizing moiety mediates transit across cellular membranes via an ENT2 transporter. In some embodiments, the internalizing moiety helps the chimeric polypeptide effectively and efficiently transit cellular membranes. In some embodiments, the internalizing moiety transits cellular membranes via an equilibrative nucleoside (ENT) transporter. In some embodiments, the internalizing moiety transits cellular membranes via an ENT1, ENT2, ENT3 or ENT4 transporter. In some embodiments, the internalizing moiety transits cellular membranes via an equilibrative nucleoside transporter 2 (ENT2) and/or ENT3 transporter. In some embodiments, the internalizing moiety promotes delivery into muscle (e.g., cardiac, diaphragm, or skeletal muscle), retinal, or neuronal (e.g., brain) cells. For any of the foregoing, in certain embodiments, the internalizing moiety is internalized into the cytoplasm. In certain embodiments, the internalizing moiety is internalized into the nucleus or lysosomes.

In certain embodiments, the internalizing moiety is an antibody or antibody fragment that binds DNA. In certain embodiments, the internalizing moiety is any of the antibody or antibody fragments described herein. In other words, in certain embodiments, the antibody or antibody fragment (e.g., antibody fragment comprising an antigen binding fragment) binds DNA. In certain embodiments, DNA binding ability is measured versus a double stranded DNA substrate. In certain embodiments, the internalizing moiety is an antibody or antibody fragment that binds DNA and/or can transit cellular membranes via ENT2. In certain embodiments, the internalizing moiety binds a DNA bubble.

In certain embodiments, the internalizing moiety is capable of binding polynucleotides. In certain embodiments, the internalizing moiety is capable of binding DNA. In certain embodiments, the internalizing moiety is an antibody capable of binding DNA. In certain embodiments, the internalizing moiety is capable of binding DNA with a K_{D} of less than 1 μM. In certain embodiments, the internalizing moiety is capable of binding DNA with a K_{D} of less than 500 nM, less than 100 nM, less than 75 nM, less than 50 nM, or even less than 30 nM, less than 20 nM, less than 10 nM, or even less than 1 nM. K_{D} can be measured using Surface Plasmon Resonance (SPR) or Quartz Crystal Microbalance (QCM), in accordance with currently standard methods. By way of example, an antibody or antibody fragment (e.g., a 3E10 antibody or antibody fragment), including an antibody or antibody fragment comprising a VH having the amino acid sequence set forth in SEQ ID NO: 17 and a VL having an amino acid sequence set forth in SEQ ID NO: 18 is known to bind DNA with a K_{D} of less than 100 nM. Thus, in certain embodiments, an internalizing moiety for use in the chimeric polypeptides of the disclosure is an antibody or antibody fragment (e.g., an antigen binding fragment) that can transit cellular membranes into the cytoplasm and binds to DNA. This is also exemplary of an anti-DNA antibody. In certain embodiments, an internalizing moiety for use herein is an anti-DNA antibody or antigen binding fragment thereof. In certain embodiments, an internalizing moiety of the disclosure, such as an antibody or antibody fragment described herein, binds a given DNA substrate with higher affinity as compared to an antibody or scFv or Fv having the VH and VL of the antibody produced by the hybridoma deposited with the ATCC under ATCC accession number PTA-2439. In certain embodiments, an internalizing moiety for use in the methods of the present disclosure is not an antibody or antibody fragment having the VH and VL of the antibody produced by the hybridoma deposited with the ATCC under ATCC accession number PTA-2439. In some embodiments, an internalizing moiety for use in the methods of the present disclosure is not a murine antibody or antibody fragment.

In fact, a full-length antibody comprising the foregoing VH and VL binds a double-stranded blunt DNA substrate with an even lower K_{D}, as evaluated by ELISA. In certain embodiments, the internalizing moiety binds double-stranded, blunt DNA, and DNA binding activity is or can be demonstrated in a binding assay using blunt DNA (see, for example, Xu et. Al. (2009) EMBO Journal 28: 568-577; Hansen et al., (2012) Sci Translation Med 4: DOI 10.1126/scitranslmed.3004385), such as by ELISA, QCM, or Biacore. In certain embodiments, the foregoing K_{D} of the antibody or antibody fragment (such as an antibody fragment comprising an antigen-binding fragment) is evaluated versus a double stranded, blunt end DNA substrate, such as the DNA substrate set forth in Xu et al. In certain embodiments, the internalizing moiety is an anti-DNA antibody. It is recognized that 3E10 and other anti-DNA antibodies may be capable of binding a variety of DNA substrates with high affinity, as has been demonstrated.

In some embodiments, any of the internalizing moieties described herein, such as any of the antibodies or antigen-binding fragments of the disclosure, is capable of binding specific nucleotide motifs present in a polynucleotide sequence. In some embodiments, the internalizing moiety is capable of binding a DNA bubble. In some embodiments, the internalizing moiety is capable of binding a DNA sequence adjacent to a DNA bubble. In some embodiments, the internalizing moiety is capable of binding a DNA sequence adjacent to a DNA bubble that is at least 3, 4, 5, 6, 7, 8, 9, 10, 11, 12, 13, 14, 15, 16, 17, 18, 19, 20, 21, 22, 23, 24, or at least 25 base pairs in length. In some embodiments, the internalizing moiety is capable of binding a 5-mer variable region adjacent to a 7-base or 11-base bubble. In certain embodiments, an internalizing moiety of the disclosure, such as an antibody or antibody fragment described herein, binds a given DNA substrate with higher affinity as compared to an antibody or scFv or Fv having the VH and VL of the antibody produced by the hybridoma deposited with the ATCC under ATCC accession number PTA-2439. In certain embodiments, an internalizing moiety for use in the methods of the present disclosure is not an antibody or antibody fragment having the VH and VL of the antibody produced by the hybridoma deposited with the ATCC under ATCC accession number PTA-2439. In some embodiments, an internalizing moiety for use in the methods of the present disclosure is not a murine antibody or antibody fragment.

In some embodiments, any of the internalizing moieties described herein bind DNA at DNA response elements. In some embodiments, the internalizing moieties bind DNA response elements to prevent transcription factors or proteins from binding to the elements. In some embodiments, the internalizing moieties block or inhibit transcription.

In certain aspects, any of the internalizing moieties described herein bind DNA at DNA repair sites. In some embodiments, the internalizing moiety binds a DNA bubble formed at a DNA repair site. In some embodiments, the internalizing moiety binds DNA at a DNA repair site wherein the DNA repair site is present as the result of DNA damage due to chemotherapeutic treatment. In some embodiments, the chemotherapeutic treatment is treatment with a DNA cross-linker (e.g., a platin such as cisplatin, carboplatin, oxaliplatin or an active analog thereof), an inhibitor of DNA synthesis (e.g., methotrexate or an active analog thereof), a topoisomerase poison (e.g., doxorubicin, daunorubicin, or an active analog thereof), a DNA alkylating agent (e.g., a nitrosurea, triazene compound or an active analog thereof), and/or an antimetabolite (e.g., a pyrimidine analog such as 5-fluorouracil or an active analog thereof).

In some embodiments, any of the internalizing moieties of the disclosure are capable of binding DNA at DNA sites independent of DNA repair sites.

In certain aspects, an internalizing moiety may comprise an antibody, including a monoclonal antibody, a polyclonal antibody, and a humanized antibody. In some embodiments, the internalizing moiety is a full-length antibody. In some embodiments, internalizing moieties may comprise antibody fragments, derivatives or analogs thereof, including without limitation: antibody fragments comprising antigen binding fragments (e.g., Fv fragments, single chain Fv (scFv) fragments, Fab fragments, Fab′ fragments, F(ab′)2 fragments), single domain antibodies, camelized antibodies and antibody fragments, humanized antibodies and antibody fragments, human antibodies and antibody fragments, and multivalent versions of the foregoing; multivalent internalizing moieties including without limitation: Fv fragments, single chain Fv (scFv) fragments, Fab′ fragments, F(ab′)2 fragments, single domain antibodies, camelized antibodies and antibody fragments, humanized antibodies and antibody fragments, human antibodies and antibody fragments, and multivalent versions of the foregoing; multivalent internalizing moieties including without limitation: monospecific or bispecific antibodies, such as disulfide stabilized Fv fragments, scFv tandems ((scFv)₂ fragments), diabodies, tribodies or tetrabodies, which typically are covalently linked or otherwise stabilized (i.e., leucine zipper or helix stabilized) scFv fragments; receptor molecules which naturally interact with a desired target molecule. In some embodiments, the antibodies or variants thereof may be chimeric, e.g., they may include variable heavy or light regions from the murine 3E10 antibody, but may include constant regions from an antibody of another species (e.g., a human). In some embodiments, the antibodies or variants thereof may comprise a constant region that is a hybrid of several different antibody subclass constant domains (e.g., any combination of IgG1, IgG2a, IgG2b, IgG3 and IgG4, from any species or combination of species). In some embodiments, the antibodies or variants thereof (e.g., the internalizing moiety) comprise the following constant domain scheme: IgG2a CH1-IgG1 hinge-IgG1 CH2-CH3, for example, any of the foregoing may be human IgG or murine IgG. Other suitable combinations are also contemplated. In other embodiments, the antibody comprises a full-length antibody and the CH1, hinge, CH2, and CH3 is from the same constant domain subclass (e.g., IgG1). In some embodiments, the antibodies or variants thereof are antibody fragments (e.g., the internalizing moiety is an antibody fragment comprising an antigen binding fragment; e.g., the internalizing moiety is an antigen binding fragment) comprising a portion of the constant domain of an immunoglobulin, for example, the following constant domain scheme: IgG2a CH1-IgG1 upper hinge. In some embodiments, the antibodies or variants thereof are antibody fragments that comprise a sequence that is at least 85%, 90%, 91%, 92%, 93%, 94%, 95%, 96%, 97%, 98%, 99% or 100% identical to the amino acid sequence of SEQ ID NO: 8.

In some embodiments, the antibodies or variants thereof comprise a kappa constant domain (e.g., of the Km3 allotype). In some embodiments, the antibodies or variants thereof are antibody fragments that comprise a sequence that is at least 85%, 90%, 91%, 92%, 93%, 94%, 95%, 96%, 97%, 98%, 99% or 100% SEQ ID NO: 9. Heavy chain constant domains (whether for a full-length antibody or for an antibody fragment (e.g., an antigen binding fragment) comprising an amino acid substitution, relative to native IgG domains, to decrease effector function and/or facilitate production are included within the scope of antibodies and antigen binding fragments. For example, one, two, three, or four amino acid substitutions in a heavy chain, relative to a native murine or human immunoglobulin constant region, such as in the hinge or CH2 domain of a heavy chain constant region.

In certain embodiments, an internalizing moiety comprises an antibody, and the heavy chain comprises a VH region, and a constant domain comprising a CH1, hinge, CH2, and CH3 domain. In certain embodiments, a heavy chain comprises a VH region, and a constant domain comprising a CH1 domain and, optionally, the upper hinge. The upper hinge may include, for example, 1, 2, 3, or 4 amino acid residues of the hinge region. In certain embodiments, the upper hinge does not include a cysteine residue. In certain embodiments, the upper hinge includes one or more consecutive residues N-terminal to a cysteine that exists in the native hinge sequence. In certain embodiments, the heavy chain comprises a CH region, and a constant domain comprising a CH1 domain and a hinge. In certain embodiments, the hinge (whether present as part of a full-length antibody or an antibody fragment) comprises a C to S substitution at a position corresponding to Kabat position 222 (e.g., a C222S in the hinge, where the variation is at a position corresponding to Kabat position 222). In other words, in certain embodiments, the internalizing moiety comprises a serine residue, rather than a cysteine residue, in a hinge domain at a position corresponding to Kabat 222. In certain embodiments, the heavy chain comprises a constant domain comprising a CH1, hinge, CH2 and, optionally CH3 domain. In certain embodiments, a CH2 domain comprises an N to Q substitution at a position corresponding to Kabat position 297 (e.g., a N297Q in a CH2 domain, wherein the variation is at a position corresponding to Kabat position 297). In other words, in certain embodiments, the internalizing moiety comprises a glutamine, rather than an asparagine, at a position corresponding to Kabat position 297.

In some embodiments, the internalizing moiety comprises all or a portion of the Fc region of an immunoglobulin. In other words, in addition to an antigen binding portion, in certain embodiments, the internalizing moiety comprises all or a portion of a heavy chain constant region of an immunoglobulin (e.g., one or two polypeptide chains of a heavy chain constant region. As is known, each immunoglobulin heavy chain constant region comprises four or five domains. The domains are named sequentially as follows: CH1-hinge-CH2-CH3(-CH4). The DNA sequences of the heavy chain domains have cross-homology among the immunoglobulin classes, e.g., the CH2 domain of IgG is homologous to the CH2 domain of IgA and IgD, and to the CH3 domain of IgM and IgE. As used herein, the term, “immunoglobulin Fc region” is understood to mean the carboxyl-terminal portion of an immunoglobulin chain constant region, preferably an immunoglobulin heavy chain constant region, or a portion thereof. For example, an immunoglobulin Fc region may comprise 1) a CH1 domain, a CH2 domain, and a CH3 domain, 2) a CH1 domain and a CH2 domain, 3) a CH1 domain and a CH3 domain, 4) a CH2 domain and a CH3 domain, or 5) a combination of two or more domains and an immunoglobulin hinge region, or a portion of a hinger (e.g., an upper hinge). In certain embodiments, an internalizing moiety further comprises a light chain constant region (CL).

In some embodiments, the Fc portion of any of the internalizing moieties described herein has been modified such that it does not induce antibody-dependent cell-mediated cytotoxicity (ADCC). In some embodiments, the Fc portion has been modified such that it does not bind complement. In certain embodiments, a CH2 domain of the Fc portion comprises an N to Q substitution at a position corresponding to Kabat position 297 (e.g., a N297Q in a CH2 domain, wherein the variation is at a position corresponding to Kabat position 297). In other words, in certain embodiments, the internalizing moiety comprises a glutamine, rather than an asparagine, at a position corresponding to Kabat position 297.

In one embodiment, the class of immunoglobulin from which the heavy chain constant region is derived is IgG (Igγ) (γ subclasses 1, 2, 3, or 4). Other classes of immunoglobulin, IgA (Igα), IgD (Igδ), IgE (IgE) and IgM (Igμ), may be used. The choice of particular immunoglobulin heavy chain constant region sequences from certain immunoglobulin classes and subclasses to achieve a particular result is considered to be within the level of skill in the art. The portion of the DNA construct encoding the immunoglobulin Fc region preferably comprises at least a portion of a hinge domain, and preferably at least a portion of a CH₃ domain of Fc γ or the homologous domains in any of IgA, IgD, IgE, or IgM. Furthermore, it is contemplated that substitution or deletion of amino acids within the immunoglobulin heavy chain constant regions may be useful in the practice of the disclosure. One example would be to introduce amino acid substitutions in the upper CH2 region to create a Fc variant with reduced affinity for Fc receptors (Cole et al. (1997) J. Immunol. 159:3613). One of ordinary skill in the art can prepare such constructs using well known molecular biology techniques.

In some embodiments, the internalizing moiety is any peptide or antibody-like protein having the complementarity determining regions (CDRs) of the 3E10 antibody sequence, or of an antibody that binds the same epitope (e.g., the same target, such as DNA) as 3E10. Also, transgenic mice, or other mammals, may be used to express humanized or human antibodies. Such humanization may be partial or complete.

In certain embodiments, the internalizing moiety comprises the monoclonal antibody 3E10 or an antigen binding fragment thereof. In other embodiments, the internalizing moiety comprises an antibody or an antigen binding fragment thereof, such as any of the antigen binding fragments described herein. For example, the antibody or antigen binding fragment thereof may be monoclonal antibody 3E10, or a variant thereof that retains cell penetrating activity, or an antigen binding fragment of 3E10 or said 3E10 variant. Additionally, the antibody or antigen binding fragment thereof may be an antibody that binds to the same epitope (e.g., target, such as DNA) as 3E10, or an antibody that has substantially the same cell penetrating activity as 3E10, or an antigen binding fragment thereof. These are exemplary of agents that can transit cells via ENT2. In certain embodiments, the internalizing moiety is capable of binding polynucleotides. In certain embodiments, the internalizing moiety is capable of binding DNA, such as double-stranded blunt DNA. In certain embodiments, the internalizing moiety is capable of binding DNA with a K_{D} of less than 100 nM. In certain embodiments, the internalizing moiety is capable of binding DNA with a K_{D} of less than 100 nM, less than 75 nM, less than 50 nM, or even less than 30 nM. K_{D} is determined using SPR or QCM or ELISA, according to manufacturer's instructions and current practice. In some embodiments, K_{D} is determined using a fluorescence polarization assay.

In certain embodiments, the antigen binding fragment is an Fv or scFv fragment thereof. Monoclonal antibody 3E10 can be produced by a hybridoma 3E10 placed permanently on deposit with the American Type Culture Collection (ATCC) under ATCC accession number PTA-2439 and is disclosed in U.S. Pat. No. 7,189,396. This antibody has been shown to bind DNA. Additionally or alternatively, the 3E10 antibody can be produced by expressing in a host cell nucleotide sequences encoding the heavy and light chains of the 3E10 antibody. The term “3E10 antibody” or “monoclonal antibody 3E10” are used to refer to the antibody, regardless of the method used to produce the antibody. Similarly, when referring to variants or antigen-binding fragments of 3E10, such terms are used without reference to the manner in which the antibody was produced. At this point, 3E10 is generally not produced by the hybridoma but is produced recombinantly. Thus, in the context of the present application, 3E10 antibody, unless otherwise specified, will refer to an antibody having the sequence of the hybridoma or comprising a variable heavy chain domain comprising the amino acid sequence set forth in SEQ ID NO: 17 (which has a one amino acid substitution relative to that of the 3E10 antibody deposited with the ATCC (D31N in CDRH1), as described herein) and the variable light chain domain comprising the amino acid sequence set forth in SEQ ID NO: 18, and antibody fragments thereof. The internalizing moiety may also comprise variants of mAb 3E10, such as variants of 3E10 which retain the same cell penetration characteristics as mAb 3E10, as well as variants modified by mutation to improve the utility thereof (e.g., improved ability to target specific cell types, improved ability to penetrate the cell membrane, improved ability to localize to the cellular DNA, convenient site for conjugation, and the like). Such variants include variants wherein one or more conservative or non-conservative substitutions are introduced into the heavy chain, the light chain and/or the constant region(s) of the antibody. Such variants include humanized versions of 3E10 or a 3E10 variant, particularly those with improved activity or utility, as provided herein. In some embodiments, the light chain or heavy chain may be modified at the N-terminus or C-terminus. Similarly, the foregoing description of variants applies to antigen binding fragments. Any of these antibodies, variants, or fragments may be made recombinantly by expression of the nucleotide sequence(s) in a host cell.

The internalizing moiety may also include mutants of mAb 3E10, such as variants of 3E10 which retain the same or substantially the same cell penetration characteristics as mAb 3E10, as well as variants modified by mutation to improve the utility thereof (e.g., improved ability to target specific cell types, improved ability to penetrate the cell membrane, improved ability to localize to the cellular DNA, improved binding affinity, and the like). Such mutants include variants wherein one or more conservative substitutions are introduced into the heavy chain, the light chain and/or the constant region(s) of the antibody. Numerous variants of mAb 3E10 have been characterized in, e.g., U.S. Pat. No. 7,189,396 and WO 2008/091911, the teachings of which are incorporated by reference herein in their entirety.

In certain embodiments, the internalizing moiety comprises an antibody or antigen binding fragment comprising an VH domain comprising an amino acid sequence at least 80%, 85%, 90%, 95%, 96%, 97%, 99%, or 100% identical to SEQ ID NO: 17 and/or a VL domain comprising an amino acid sequence at least 85%, 90%, 95%, 96%, 97%, 99%, or 100% identical to SEQ ID NO: 18, or a humanized variant thereof. It is understood that, when a signal sequence is included for expression of an antibody or antibody fragment, that signal sequence is generally cleaved and not presented in the finished chimeric polypeptide (e.g., the signal sequence is generally cleaved and present only transiently during protein production). Such internalizing moieties transit, in certain embodiments, cells via ENT2 and/or bind DNA. In certain embodiments, an internalizing moiety for use in the methods of the present disclosure (or an antibody or antigen binding fragment for such use) is not an antibody or antibody fragment having the VH and VL of the antibody produced by the hybridoma deposited with the ATCC under ATCC accession number PTA-2439. In some embodiments, an internalizing moiety for use in the methods of the present disclosure (or an antibody or antigen binding fragment for such use) is not an antibody or antibody fragment having a VH comprising the amino acid sequence set forth in SEQ ID NO: 17 and a VL comprising the amino acid sequence set forth in SEQ ID NO: 18.

In certain embodiments, the internalizing moiety is capable of binding polynucleotides. In certain embodiments, the internalizing moiety is capable of binding (specifically binding) DNA. In certain embodiments, the internalizing moiety is capable of binding DNA with a K_{D} of less than 100 nM. In certain embodiments, the internalizing moiety is capable of binding DNA with a K_{D} of less than 50 nM. In certain embodiments, the internalizing moiety is an anti-DNA antibody, such as an antibody or antigen binding fragment that binds double-stranded blunt DNA. In certain embodiments, the internalizing moiety is an anti-DNA antibody or antigen binding fragment (thereof), where K_{D} is evaluated versus a double stranded DNA substrate, such as provided herein.

In certain embodiments, the internalizing moiety is an antigen binding fragment, such as a single chain Fv of 3E10 (scFv) comprising SEQ ID NOs: 17 and 18. In certain embodiments, the internalizing moiety comprises a single chain Fv of 3E10 (or another antigen binding fragment), and the amino acid sequence of the V_{H} domain is at least 90%, 95%, 96%, 97%, 98%, 99%, or 100% identical to SEQ ID NO: 17, and amino acid sequence of the V_{L} domain is at least 90%, 95%, 96%, 97%, 98%, 99%, or 100% identical to SEQ ID NO: 18. The variant 3E10 or fragment thereof retains the function of an internalizing moiety. When the internalizing moiety is an scFv, the VH and VL domains are typically connected via a linker, such as a gly/ser linker. The VH domain may be N-terminal to the VL domain or vice versa.

In certain embodiments, the internalizing moiety is an antigen binding fragment, such as a Fab comprising a VH and a VL. In certain embodiments, the internalizing moiety is a Fab (or another antigen binding fragment, such as a Fab′), and the amino acid sequence of the V_{H} domain is at least 90%, 95%, 96%, 97%, 98%, 99%, or 100% identical to SEQ ID NO: 17. In certain embodiments, the internalizing moiety is a Fab (or another antigen binding fragment, such as a Fab′), and the amino acid sequence of the V_{L} domain is at least 90%, 95%, 96%, 97%, 98%, 99%, or 100% identical to SEQ ID NO: 18. Our VH and VL domains, or combinations thereof, described herein are similarly contemplated. In certain embodiments, when the internalizing moiety is a Fab the heavy chain comprises a CH1 domain and an upper hinge of an immunoglobulin constant region. In certain embodiments, the upper hinge comprises a substitution, relative to a native immunoglobulin constant region, such as to decrease effector function and/or to eliminate a cysteine (e.g., a C to S). In certain embodiments, the upper hinge does not include a cysteine.

In certain embodiments, an internalizing moiety for use in the methods of the present disclosure (or an antibody or antigen binding fragment for such use) is not an antibody or antibody fragment having the VH and VL of the antibody produced by the hybridoma deposited with the ATCC under ATCC accession number PTA-2439. In some embodiments, an internalizing moiety for use in the methods of the present disclosure (or an antibody or antigen binding fragment for such use) is not an antibody or antibody fragment having a VH comprising the amino acid sequence set forth in SEQ ID NO: 17 and a VL comprising the amino acid sequence set forth in SEQ ID NO: 18.

In certain embodiments, the constant domain of the antibody or antibody fragment (e.g., antigen binding fragment) comprises all or a portion of a human Fc domain. In certain embodiments, the internalizing moiety is a full-length antibody, and the constant domain of the antibody comprises a CH1, hinge, CH2 and CH3 domain. In certain embodiments, the constant domain comprises one or more substitutions, relative to a native immunoglobulin, that reduce effector function. Optionally, in certain embodiments, such a constant domain may include one or more (e.g., 1 substitution, 2 substitutions, 3 substitutions) substitutions in the heavy chain constant domain, such as in the hinge and/or CH2 domains, such as to reduce effector function. Such substitutions are known in the art.

In certain embodiments, the internalizing moiety is an antigen binding fragment—a fragment of an antibody comprising an antigen binding fragment. Suitable such fragments of antibodies, such as scFv, Fab, Fab′ and the like are described herein. In certain embodiments, the internalizing moiety is an antigen binding fragment or a full-length antibody. In certain embodiments, the internalizing moiety comprises a light chain comprising a constant region (CL). In certain embodiments, the internalizing moiety comprises a heavy chain comprising a constant region, wherein the constant region comprises a CH1 domain. In certain embodiments, the internalizing moiety comprises a heavy chain comprising a constant region and a light chain comprising a constant region, wherein the heavy chain constant region comprises a CH1 domain. Optionally, the internalizing moiety may further comprise a heavy chain constant region comprising all or a portion of a hinge (e.g., an upper hinge or more than the upper hinge). Optionally, the internalizing moiety may further comprise a heavy chain comprising a CH2 and/or CH3 domain.

In some embodiments, the internalizing moiety comprises one or more of the CDRs of the 3E10 antibody. In certain embodiments, the internalizing moiety comprises one or more of the CDRs of a 3E10 antibody comprising the amino acid sequence of a V_{H} domain that is identical to SEQ ID NO: 17 and the amino acid sequence of a V_{L} domain that is identical to SEQ ID NO: 18. The CDRs of the 3E10 antibody may be determined using any of the CDR identification schemes available in the art. For example, in some embodiments, the CDRs of the 3E10 antibody are defined according to the Kabat definition as set forth in Kabat et al. Sequences of Proteins of Immunological Interest, 5th Ed. Public Health Service, National Institutes of Health, Bethesda, Md. (1991). In other embodiments, the CDRs of the 3E10 antibody are defined according to Chothia et al., 1987, J Mol Biol. 196: 901-917 and Chothia et al., 1989, Nature. 342:877-883. In other embodiments, the CDRs of the 3E10 antibody are defined according to the international ImMunoGeneTics database (IMGT) as set forth in LeFranc et al., 2003, Development and Comparative Immunology, 27: 55-77. In other embodiments, the CDRs of the 3E10 antibody are defined according to Honegger A, Pluckthun A., 2001, J Mol Biol., 309:657-670. In some embodiments, the CDRs of the 3E10 antibody are defined according to any of the CDR identification schemes discussed in Kunik et al., 2012, PLoS Comput Biol. 8(2): e1002388. In order to number residues of a 3E10 antibody for the purpose of identifying CDRs according to any of the CDR identification schemes known in the art, one may align the 3E10 antibody at regions of homology of the sequence of the antibody with a “standard” numbered sequence known in the art for the elected CDR identification scheme. Maximal alignment of framework residues frequently requires the insertion of “spacer” residues in the numbering system, to be used for the Fv region. In addition, the identity of certain individual residues at any given site number may vary from antibody chain to antibody chain due to interspecies or allelic divergence.

In certain embodiments, the internalizing moiety comprises at least 1, 2, 3, 4, or 5 of the CDRs of 3E10 as determined using the Kabat CDR identification scheme (e.g., the CDRs set forth in SEQ ID NOs: 19-24; the internalizing moiety is an antibody or antigen binding fragment thereof comprising a heavy chain comprising CDR1, CDR2, and CDR 3, as set forth in SEQ ID NOs: 19-21, respectively, and a light chain comprising CDR1, CDR2, and CDR3, as set forth in SEQ ID NOs: 22-24, respectively; e.g., and these CDRs in the internalizing moiety are as determined using the Kabat scheme). In other embodiments, the internalizing moiety comprises at least 1, 2, 3, 4 or 5 of the CDRs of 3E10 as determined using the IMGT identification scheme (e.g., the CDRs set forth in SEQ ID NOs: 25-30; the internalizing moiety is an antibody or antigen binding fragment thereof comprising a heavy chain comprising CDR1, CDR2, and CDR 3, as set forth in SEQ ID NOs: 25-27, respectively, and a light chain comprising CDR1, CDR2, and CDR3, as set forth in SEQ ID NOs: 28-30, respectively; e.g., and these CDRs in the internalizing moiety are as determined using the IMGT identification scheme). In certain embodiments, the internalizing moiety comprises all six CDRs of 3E10 as determined using the Kabat CDR identification scheme (e.g., comprises SEQ ID NOs 19-24). In other embodiments, the internalizing moiety comprises all six CDRS of 3E10 as determined using the IMGT identification scheme (e.g., which are set forth as SEQ ID NOs: 25-30). For any of the foregoing, in certain embodiments, the internalizing moiety is an antibody that binds the same epitope (e.g., the same target, such as DNA) as 3E10 and/or the internalizing moiety competes with 3E10 for binding to antigen. Exemplary internalizing moieties target and transit cells via ENT2. Exemplary internalizing moieties comprise antibodies or antigen binding fragments that bind DNA, such as double stranded blunt DNA.

In certain embodiments, the internalizing moiety comprising an antibody fragment, and the antibody fragment comprises an antigen binding fragment, such as an Fab or Fab′. In other words, in certain embodiments, the internalizing moiety comprises an Fab or Fab′.

In certain embodiments, the internalizing moiety competes with binding for a DNA substrate, such as double-stranded blunt DNA, with an antibody (or antigen-binding fragment) of the antibody produced by hybridoma 3E10 placed permanently on deposit with the American Type Culture Collection (ATCC) under ATCC accession number PTA-2439.

Preparation of antibodies or fragments thereof (e.g., a single chain Fv fragment encoded by V_{H}-linker-V_{L} or V_{L}-linker-V_{H} or a Fab) is well known in the art. In particular, methods of recombinant production of mAb 3E10 antibody fragments have been described in WO 2008/091911. Further, methods of generating scFv fragments of antibodies or Fabs are well known in the art. When recombinantly producing an antibody or antibody fragment, a linker may be used. For example, typical surface amino acids in flexible protein regions include Gly, Asn and Ser. One exemplary linker is provided in SEQ ID NO: 5 or 16. Permutations of amino acid sequences containing Gly, Asn and Ser would be expected to satisfy the criteria (e.g., flexible with minimal hydrophobic or charged character) for a linker sequence. Another exemplary linker is of the formula (G₄S)n, wherein n is an integer from 1-10, such as 2, 3, or 4. Other near neutral amino acids, such as Thr and Ala, can also be used in the linker sequence.

Preparation of antibodies may be accomplished by any number of well-known methods for generating monoclonal antibodies. These methods typically include the step of immunization of animals, typically mice, with a desired immunogen (e.g., a desired target molecule or fragment thereof). Once the mice have been immunized, and preferably boosted one or more times with the desired immunogen(s), monoclonal antibody-producing hybridomas may be prepared and screened according to well known methods (see, for example, Kuby, Janis, Immunology, Third Edition, pp. 131-139, W.H. Freeman & Co. (1997), for a general overview of monoclonal antibody production, that portion of which is incorporated herein by reference). Over the past several decades, antibody production has become extremely robust. In vitro methods that combine antibody recognition and phage display techniques allow one to amplify and select antibodies with very specific binding capabilities. See, for example, Holt, L. J. et al., “The Use of Recombinant Antibodies in Proteomics,” Current Opinion in Biotechnology, 2000, 11:445-449, incorporated herein by reference. These methods typically are much less cumbersome than preparation of hybridomas by traditional monoclonal antibody preparation methods. In one embodiment, phage display technology may be used to generate an internalizing moiety specific for a desired target molecule. An immune response to a selected immunogen is elicited in an animal (such as a mouse, rabbit, goat or other animal) and the response is boosted to expand the immunogen-specific B-cell population. Messenger RNA is isolated from those B-cells, or optionally a monoclonal or polyclonal hybridoma population. The mRNA is reverse-transcribed by known methods using either a poly-A primer or murine immunoglobulin-specific primer(s), typically specific to sequences adjacent to the desired V_{H} and V_{L} chains, to yield cDNA. The desired V_{H} and V_{L} chains are amplified by polymerase chain reaction (PCR) typically using VH and V_{L} specific primer sets, and are ligated together, separated by a linker. V_{H} and V_{L} specific primer sets are commercially available, for instance from Stratagene, Inc. of La Jolla, Calif. Assembled V_{H}-linker-V_{L} product (encoding an scFv fragment) is selected for and amplified by PCR. Restriction sites are introduced into the ends of the V_{H}-linker-V_{L} product by PCR with primers including restriction sites and the scFv fragment is inserted into a suitable expression vector (typically a plasmid) for phage display. Other fragments, such as a Fab′ fragment, may be cloned into phage display vectors for surface expression on phage particles. The phage may be any phage, such as lambda, but typically is a filamentous phage, such as fd and M13, typically M13.

In certain embodiments, an antibody or antibody fragment is made recombinantly in a host cell. In other words, once the sequence of the antibody is known (for example, using the methods described above), the antibody can be made recombinantly using standard techniques.

In certain embodiments, the internalizing moieties may be modified to make them more resistant to cleavage by proteases. For example, the stability of an internalizing moiety comprising a polypeptide may be increased by substituting one or more of the naturally occurring amino acids in the (L) configuration with D-amino acids. In various embodiments, at least 1%, 5%, 10%, 20%, 50%, 80%, 90% or 100% of the amino acid residues of internalizing moiety may be of the D configuration. The switch from L to D amino acids neutralizes the digestion capabilities of many of the ubiquitous peptidases found in the digestive tract. Alternatively, enhanced stability of an internalizing moiety comprising a peptide bond may be achieved by the introduction of modifications of the traditional peptide linkages. For example, the introduction of a cyclic ring within the polypeptide backbone may confer enhanced stability in order to circumvent the effect of many proteolytic enzymes known to digest polypeptides in the stomach or other digestive organs and in serum. In still other embodiments, enhanced stability of an internalizing moiety may be achieved by intercalating one or more dextrorotatory amino acids (such as, dextrorotatory phenylalanine or dextrorotatory tryptophan) between the amino acids of internalizing moiety. In exemplary embodiments, such modifications increase the protease resistance of an internalizing moiety without affecting the activity or specificity of the interaction with a desired target molecule.

The disclosure contemplates the use of internalizing moieties (including antibodies or antigen binding fragments of the disclosure) described based on any combination of any of the foregoing or following structural and/or functional characteristics. Any such internalizing moieties, such as antibodies or antigen-binding fragments, are considered antibodies and antigen binding fragments of the disclosure and can be used for any of the uses or methods described herein.

Further Examples of Antibodies or Antigen-Binding Fragments, Such as Humanized Antibodies or Antigen Binding Fragments

In some embodiments, the disclosure provides any of the antibodies or antigen-binding fragments disclosed herein, wherein the antibody or antigen-binding fragment is humanized. In other words, one class of internalizing moiety, such as antibody or antigen binding fragment, is a humanized antibody or antigen binding fragment. Such internalizing moiety may be humanized in whole or in part. Numerous examples of such humanized internalizing moieties are provided herein and are also described in WO 2015/106290, which is incorporated herein in its entirety.

In one embodiment, the disclosure provides an antibody or antigen-binding fragment comprising a humanized antibody or antigen-binding fragment, wherein the humanized antibody or antigen-binding fragment comprises a light chain variable (VL) domain and a heavy chain variable (VH) domain; wherein the V_{H} domain is humanized and comprises:

a VH CDR1 having the amino acid sequence of SEQ ID NO: 10;

a VH CDR2 having the amino acid sequence of SEQ ID NO: 11; and

a VH CDR3 having the amino acid sequence of SEQ ID NO: 12;
and the VL is humanized and comprises:

a VL CDR1 having the amino acid sequence of SEQ ID NO: 13;

a VL CDR2 having the amino acid sequence of SEQ ID NO: 14; and

a VL CDR3 having the amino acid sequence of SEQ ID NO: 15;

which CDRs are in accordance with the IMGT system, and wherein the humanized antibody or antigen-binding fragment has increased DNA binding and/or cell penetration, relative to that of a murine 3E10 antibody comprising a light chain variable (VL) domain having the amino acid sequence of SEQ ID NO: 18 and a heavy chain variable (VH) domain having the amino acid sequence of SEQ ID NO: 17. In certain embodiments, when comparing an antibody or antigen-binding fragment of the disclosure to a murine antibody or to another humanized antibody, the suitable comparison is between two proteins of the same structure (e.g., comparing a full-length antibody to another full-length antibody or comparing an Fab to another Fab). However, in other embodiments, the comparison is to an scFv or Fv of the murine antibody as a constant basis for comparison.

In some embodiments, an asparagine is mutated to another amino acid residue in the VH or VL domains in order to reduce N-linked glycosylation of the humanized antibody or antibody fragment. This humanized antibody or antibody fragment is based on a murine parent antibody—specifically a murine 3E10 antibody comprising a heavy chain and a light chain, wherein the light chain comprises a VL comprising the amino acid sequence of SEQ ID NO: 18 and the heavy chain comprises a VH comprising the amino acid sequence of SEQ ID NO: 17. In preferred embodiments, the internalizing moieties and fragments are associated with at least the cell-penetration properties associated with the murine 3E10 antibody (e.g., retain at least 75%, 80%, 85%, 90%, 95%, or greater than 95%) of the cell penetration properties. In certain embodiments, the humanized antibody or antibody fragment has one or more preferable cell penetration characteristics, such as improved penetration efficiency. In other embodiments, the humanized antibody or antibody fragment has improved DNA binding activity and/or a different range of DNA substrate affinity or specificity.

As used herein, the term “fragment” or “antigen-binding fragment” of a humanized antibody moiety or “antigen binding fragment” includes any fragment of a humanized internalizing moiety that retains at least the cell-penetration and/or DNA binding properties associated with the murine 3E10 antibody. In this application, the terms “fragment” and “antigen binding fragment” are used interchangeably. Exemplary antibody fragments include scFv fragments, Fab fragments (e.g., Fab′ or F(ab′)2), and the like.

In some embodiments, the humanized internalizing moiety (e.g., the humanized antibody and antigen binding fragments of the disclosure) is not directly fused to any heterologous agent or not fused or otherwise linked to a therapeutic or toxic heterologous agent. However, in such embodiments, and as described in greater detail below, the internalizing moiety may still be post-translationally modified (e.g., glycosylated or) and/or provided as part of a composition.

In other embodiments, the humanized internalizing moiety (e.g., the antibodies or antigen binding fragments of the disclosure, such as humanized antibodies or antibody binding fragments) is fused to a heterologous agent or a therapeutic or toxic heterologous agent. In some embodiments, the internalizing moiety effects delivery of a heterologous agent into a cell (i.e., penetrate desired cell; transport across a cellular membrane; deliver across cellular membranes to, at least, the cytoplasm). In certain embodiments, this disclosure relates to an internalizing moiety which promotes delivery of a heterologous agent into muscle, liver and/or neuronal cells, as well as certain other cell types. This portion promotes entry of the conjugate into cells. Like the murine, parental antibody, the humanized antibody and antigen binding fragments of the disclosure promote entry into cells via an ENT transporter, such as an ENT2 transporter and/or an ENT3 transporter. Without being bound by theory, ENT2 is expressed preferentially in certain cell types, including muscle (skeletal and cardiac), neuronal, retinal, and/or liver cells. Accordingly, conjugates (e.g., conjugates in which a humanized antibody or antigen binding fragment of the disclosure is conjugated to a heterologous agent) are delivered into cells, but generally not ubiquitously. Rather, the conjugates may be delivered with some level of enrichment for particular tissues, including skeletal muscle, cardiac muscle, diaphragm, liver, retina, and neurons.

In certain embodiments, the internalizing moiety is capable of binding polynucleotides (e.g., a target/antigen for an antibody of the disclosure is DNA). This is consistent with the properties of the 3E10 antibody which is known to bind DNA (e.g., to specifically bind DNA). In certain embodiments, the internalizing moiety is capable of binding DNA. In certain embodiments, the internalizing moiety is capable of binding DNA with a K_{D} of less than 100 nM. In certain embodiments, the internalizing moiety is capable of binding DNA (e.g., single stranded DNA or blunt double stranded DNA) with a K_{D} of less than 500 nM, less than 100 nM, less than 75 nM, less than 50 nM, or even less than 30 nM, less than 20 nM, less than 10 nM, or even less than 1 nM. K_{D} can be measured using Surface Plasmon Resonance (SPR) or Quartz Crystal Microbalance (QCM), or by ELISA, in accordance with currently standard methods. By way of example, an antibody or antibody fragment comprising a VH having the amino acid sequence set forth in SEQ ID NO: 3 and a VL having an amino acid sequence set forth in SEQ ID NO: 4 specifically binds DNA with a K_{D} of less than 100 nM, and is an example of an anti-DNA antibody. In certain embodiments, the internalizing moiety binds double-stranded, blunt DNA, and DNA binding activity is or can be demonstrated in a binding assay using blunt DNA (see, for example, Xu et. Al. (2009) EMBO Journal 28: 568-577; Hansen et al., (2012) Sci Translation Med 4: DOI 10.1126/scitranslmed.3004385), such as by ELISA, QCM, or Biacore. In certain embodiments, the internalizing moiety is an anti-DNA antibody. Thus, in certain embodiments, an internalizing moiety (e.g., an antibody or antigen binding fragment) for use alone or associated with a heterologous agent comprises an antibody or antibody fragment that can transit cellular membranes into the cytoplasm and/or the nucleus and is capable of binding to DNA. In certain embodiments, the antibody and antigen binding fragments of the disclosure, such as humanized antibodies and antigen binding fragments, are based upon a murine, parental 3E10 antibody having VH and VL domains, as described above.

Preferably, the humanized antibody has the same, substantially the same, or even improved cell penetration and/or DNA binding characteristics in comparison to the murine, parental antibody, including a murine parental antibody comprising, when present, a murine constant domain.

In certain embodiments, the antibodies and antigen binding fragments of the disclosure have the same CDRs, as defined using the IMGT system, as the murine, parent antibody (e.g., the antibody comprising a heavy chain comprising a VH comprising the amino acid sequence set forth in SEQ ID NO: 17 and a light chain comprising a VL comprising the amino acid sequence set forth in SEQ ID NO: 18). In certain embodiments, the antibodies and antigen binding fragments of the disclosure have at least one CDR of the heavy chain and/or the light chain that differs from that of the murine, parent antibody (e.g., differ at VH CDR2 and/or VL CDR2 and/or VL CDR1, according to Kabat). In some embodiments, a humanized antibody or antigen binding fragment of the disclosure comprises a V_{H} domain and a V_{L} domain comprising:

a VH CDR1 having the amino acid sequence of SEQ ID NO: 10;

a VH CDR2 having the amino acid sequence of SEQ ID NO: 11;

a VH CDR3 having the amino acid sequence of SEQ ID NO: 12;

a VL CDR1 having the amino acid sequence of SEQ ID NO: 13;

a VL CDR2 having the amino acid sequence of SEQ ID NO: 14; and

a VL CDR3 having the amino acid sequence of SEQ ID NO: 15, which CDRs are in accordance with the IMGT system.

In some embodiments, a humanized antibody or antigen binding fragment of the disclosure comprises a V_{H} domain and a V_{L} domain comprising:

a VH CDR1 having the amino acid sequence of SEQ ID NO: 19;

a VH CDR2 having the amino acid sequence of SEQ ID NO: 20; and

a VH CDR3 having the amino acid sequence of SEQ ID NO: 21, which CDRs are according to Kabat; and

a VL CDR1 having the amino acid sequence of SEQ ID NO: 13;

a VL CDR2 having the amino acid sequence of SEQ ID NO: 14; and

a VL CDR3 having the amino acid sequence of SEQ ID NO: 15, which CDRs are according to the IMGT system.

In some embodiments, a humanized antibody or antigen binding fragment of the disclosure comprises a V_{H} domain and a V_{L} domain comprising:

a VH CDR1 having the amino acid sequence of SEQ ID NO: 10;

a VH CDR2 having the amino acid sequence of SEQ ID NO: 11; and

a VH CDR3 having the amino acid sequence of SEQ ID NO: 12, which CDRs are according to the IMGT system, and

a VL CDR1 having the amino acid sequence of SEQ ID NO: 22;

a VL CDR2 having the amino acid sequence of SEQ ID NO: 23; and

a VL CDR3 having the amino acid sequence of SEQ ID NO: 24, which CDRs are according to Kabat.

In certain embodiments, an antibody or antigen binding fragment of the disclosure comprises a V_{H} domain comprising:

a VH CDR1 having the amino acid sequence of SEQ ID NO: 19;

a VH CDR2 having the amino acid sequence of SEQ ID NO: 25; and

a VH CDR3 having the amino acid sequence of SEQ ID NO: 21, which CDRs are according to the Kabat system, and

a V_{L} domain comprising

a VL CDR1 having the amino acid sequence of SEQ ID NO: 22 or 26;

a VL CDR2 having the amino acid sequence of SEQ ID NO: 27; and

a VL CDR3 having the amino acid sequence of SEQ ID NO: 24, which CDRs are according to Kabat.

As detailed throughout the application, the antibody or antigen-binding fragments of the disclosure, such as humanized antibody or antigen binding fragments, can be compared to the murine, parent antibody or to the original 3E10 antibody or antigen binding fragment thereof. Additionally or alternatively, antibodies of the disclosure (or antigen binding fragments thereof) can be compared to alternate antibodies and fragments (e.g., other humanized antibodies based on the same murine parent). In such scenarios, the comparison could be to an alternate antibody or antigen binding fragment have the foregoing 6 IMGT or Kabat CDRs, but have one or more changes in the framework regions relative to the humanized antibody or antigen-binding fragment of the disclosure. Also contemplated are antibodies or antigen binding fragments having the CDRs disclosed herein, but with one, two, three, or four amino acid substitutions in one or more CDRs (e.g., with one substitution in one CDR, with two substitution—one in each of two CDRS, or with three substitutions—one in each of three CDRs). When comparing activity, the ability and efficiency to penetrate cells, such as muscle, retinal, and/or neuronal cells, via ENT2 and/or ENT3 may be assessed. Activity will be considered comparable or substantially the same if it is approximately 70%, 75%, 80%, 85%, 90%, 95%, or greater than about 95% the activity of the murine, parental antibody. Activity is considered improved, relative to the murine, parental antibody, if a characteristic is at least about 5%, preferably at least about 10% better (e.g., approximately 105%, 110%, 115%, 120%, 125%, 130%, 150%, or greater than 150% the activity of the murine, parental antibody or an alternate humanized antibody). In certain embodiments, an activity is considered improved, relative to another antibody, if a characteristic is at least 2-fold better. In other embodiments, an activity is considered improved if a characteristic is at least 3-, 4-, 5-, 6-, 8, or 10-fold better.

In some embodiments, antibodies or humanized antibodies may comprise antibody fragments, derivatives or analogs thereof, including without limitation: antibody fragments comprising an antigen binding fragments (e.g., Fv fragments, single chain Fv (scFv) fragments, Fab fragments, Fab′ fragments, F(ab′)2 fragments, single domain antibodies, and multivalent versions of the foregoing; multivalent internalizing moieties including without limitation: Fv fragments, single chain Fv (scFv) fragments, Fab fragments, Fab′ fragments, F(ab′)2 fragments, single domain antibodies, camelized antibodies and antibody fragments, humanized antibodies and antibody fragments, human antibodies and antibody fragments, and multivalent versions of the foregoing; multivalent internalizing moieties including without limitation: monospecific or bispecific antibodies, such as disulfide stabilized Fv fragments, scFv tandems ((scFv)₂ fragments), diabodies, tribodies or tetrabodies, which typically are covalently linked or otherwise stabilized (i.e., leucine zipper or helix stabilized) scFv fragments; receptor molecules which naturally interact with a desired target molecule. In certain embodiments, the antigen-binding fragment is an scFv and a peptide linker interconnects the VH domain and the VL domain. In some embodiments, the antibodies or variants thereof may comprise a constant region that is a hybrid of several different antibody subclass constant domains (e.g., any combination of IgG1, IgG2a, IgG2b, IgG3 and IgG4).

In certain embodiments, the internalizing moiety is an antibody fragment comprising an antigen binding fragment. In other words, in certain embodiments, the internalizing moiety is not a full-length antibody but is a fragment thereof comprising an antigen binding fragment. In certain embodiments, the internalizing moiety is an scFv, Fab, Fab′, or Fab2′. In certain embodiments, the internalizing moiety is a full-length antibody comprising a heavy chain comprising a CH1, hinge, CH2, and CH3 domains, optionally substituted to reduce effector function, such as in the hinge and/or CH2 domains, as described herein. In certain embodiments, the heavy chain comprises a VH domain, and a constant domain comprising a CH1, hinge, CH2, and CH3 domain. In certain embodiments, a heavy chain comprises a VH domain, and a constant domain comprising a CH1 domain and, optionally the upper hinge. The upper hinge may include, for example, 1, 2, 3, or 4 amino acid residues of the hinge region. In certain embodiments, the upper hinge does not include a cysteine residue. In certain embodiments, the upper hinge includes one or more consecutive residues N-terminal to a cysteine that exists in the native hinge sequence. In certain embodiments, the heavy chain comprises a CH region, and a constant domain comprising a CH1 domain and a hinge. In certain embodiments, the hinge (whether present as part of a full-length antibody or an antibody fragment) comprises a C to S substitution at a position corresponding to Kabat position 222 (e.g., a C222S in the hinge, where the variation is at a position corresponding to Kabat position 222). In other words, in certain embodiments, the internalizing moiety comprises a serine residue, rather than a cysteine residue, in a hinge domain at a position corresponding to Kabat 222. In certain embodiments, the heavy chain comprises a constant domain comprising a CH1, hinge, CH2 and, optionally CH3 domain. In certain embodiments, a CH2 domain comprises an N to Q substitution at a position corresponding to Kabat position 297 (e.g., a N297Q in a CH2 domain, wherein the variation is at a position corresponding to Kabat position 297). In other words, in certain embodiments, the internalizing moiety comprises a glutamine, rather than an asparagine, at a position corresponding to Kabat position 297.

In certain embodiments, an antibody or antigen binding fragment as disclosed herein is a full-length antibody comprising CH1, hinge, CH2, and CH3 of a heavy chain constant domain and a light chain constant domain. In certain embodiments the heavy chain constant region comprises one or more of a CH1, CH2, and CH3 domains, optionally with a hinge.

Monoclonal antibody 3E10 can be produced by hybridoma 3E10 placed permanently on deposit with the American Type Culture Collection (ATCC) under ATCC accession number PTA-2439 and is disclosed in U.S. Pat. No. 7,189,396. This antibody has been shown to bind DNA. Additionally or alternatively, the 3E10 antibody can be produced by expressing in a host cell nucleotide sequences encoding the heavy and light chains of the 3E10 antibody. The term “3E10 antibody” or “monoclonal antibody 3E10” are used also herein to refer to a murine antibody (or antigen binding fragment) comprising the a VL domain comprising the amino acid sequence of SEQ ID NO: 18 and a VH domain comprising the amino acid sequence of SEQ ID NO:17, regardless of the method used to produce the antibody. Thus, in the context of the present application, 3E10 antibody will refer, unless otherwise specified, to an antibody having the sequence of the hybridoma or comprising a variable heavy chain domain comprising the amino acid sequence set forth in SEQ ID NO: 17 (which has a one amino acid substitution relative to that of the 3E10 antibody deposited with the ATCC, as described herein and previously demonstrated as retaining cell penetration and DNA binding activity) and the variable light chain domain comprising the amino acid sequence set forth in SEQ ID NO: 18. However, in the context of the present disclosure, the parent murine antibody used as the basis for humanization was an antibody comprising the VL domain comprising the amino acid sequence of SEQ ID NO: 18 and a VH domain comprising the amino acid sequence of SEQ ID NO: 17. The disclosure provides, in certain embodiments, humanized antibodies based on murine 3E10.

Similarly, when referring to variants or antigen-binding fragments of 3E10, such terms are used without reference to the manner in which the antibody was produced. At this point, 3E10 is generally produced recombinantly.

The humanized internalizing moiety may also be derived from variants of mAb 3E10, such as variants of 3E10 which retain the same cell penetration characteristics as mAb 3E10, as well as variants modified by mutation to improve the utility thereof (e.g., improved ability to target specific cell types, improved ability to penetrate the cell membrane, improved ability to localize to the cellular DNA, convenient site for conjugation, and the like). Such variants include variants wherein one or more conservative substitutions are introduced into the heavy chain, the light chain and/or the constant region(s) of the antibody. In some embodiments, the light chain or heavy chain may be modified at the N-terminus or C-terminus. Moreover, the antibody or antibody fragment may be modified to facilitate conjugation to a heterologous agent. Similarly, the foregoing description of variants applies to antigen binding fragments. Any of these antibodies, variants, or fragments may be made recombinantly by expression of the nucleotide sequence(s) in a host cell. Such internalizing moieties can transit cells via an ENT transporter, such as ENT2 and/or ENT3 and/or bind the same epitope (e.g., target, such as DNA) as 3E10.

The humanized internalizing moiety may also be derived from mutants of mAb 3E10, such as variants of 3E10 which retain the same or substantially the same cell penetration characteristics as mAb 3E10, as well as variants modified by mutation to improve the utility thereof (e.g., improved ability to target specific cell types, improved ability to penetrate the cell membrane, improved ability to localize to the cellular DNA, improved binding affinity, and the like). Such mutants include variants wherein one or more conservative substitutions are introduced into the heavy chain or the light chain. Numerous variants of mAb 3E10 have been characterized in, e.g., U.S. Pat. No. 7,189,396 and WO 2008/091911, the teachings of which are incorporated by reference herein in their entirety. In the examples provided herein, the parent, murine 3E10 comprises a VH comprising the amino acid sequence set forth in SEQ ID NO: 17 and a VL comprising the amino acid sequence set forth in SEQ ID NO: 18.

In certain embodiments, the internalizing moiety is an antigen binding fragment, such as a humanized single chain Fv (scFv). In other embodiments, the humanized antibody is a Fab′ fragment.

In some embodiments, the internalizing moiety is an antibody or antibody fragment comprising an immunoglobulin heavy chain constant region or fragment thereof. As is known, each immunoglobulin heavy chain constant region comprises four or five domains. The domains are named sequentially as follows: C_{H}1-hinge-C_{H}2-C_{H}3(-C_{H}4). The DNA sequences of the heavy chain domains have cross-homology among the immunoglobulin classes, e.g., the C_{H}2 domain of IgG is homologous to the C_{H}2 domain of IgA and IgD, and to the C_{H}3 domain of IgM and IgE. As used herein, the term, “immunoglobulin Fc region” is understood to mean the carboxyl-terminal portion of an immunoglobulin heavy chain constant region, preferably an immunoglobulin heavy chain constant region, or a portion thereof. For example, an immunoglobulin Fc region may comprise 1) a CH1 domain, a CH2 domain, and a CH3 domain, 2) a CH1 domain and a CH2 domain, 3) a CH1 domain and a CH3 domain, 4) a CH2 domain and a CH3 domain, or 5) a combination of two or more domains and an immunoglobulin hinge region. In one embodiment, the immunoglobulin Fc region comprises at least an immunoglobulin hinge region a CH2 domain and a CH3 domain, and lacks the CH1 domain. In one embodiment, the class of immunoglobulin from which the heavy chain constant region is derived is IgG (Igγ) (γ subclasses 1, 2, 3, or 4). Other classes of immunoglobulin, IgA (Igα), IgD (Igδ), IgE (IgE) and IgM (Igμ), may be used. The choice of appropriate immunoglobulin heavy chain constant regions is discussed in detail in U.S. Pat. Nos. 5,541,087, and 5,726,044. The choice of particular immunoglobulin heavy chain constant region sequences from certain immunoglobulin classes and subclasses to achieve a particular result is considered to be within the level of skill in the art. The portion of the DNA construct encoding the immunoglobulin Fc region may comprise at least a portion of a hinge domain, and preferably at least a portion of a CH3 domain of Fc γ or the homologous domains in any of IgA, IgD, IgE, or IgM. Furthermore, it is contemplated that substitution or deletion of amino acids within the immunoglobulin heavy chain constant regions may be useful in the practice of the disclosure. In certain embodiments, the constant region domains are human. In some embodiments, the Fc portion of any of the internalizing moieties described herein has been modified such that it does not induce antibody-dependent cell-mediated cytotoxicity (ADCC). In some embodiments, the Fc portion has been modified such that it does not bind complement. In certain embodiments, a CH2 domain comprises an N to Q substitution at a position corresponding to Kabat position 297 (e.g., a N297Q in a CH2 domain, wherein the variation is at a position corresponding to Kabat position 297). In other words, in certain embodiments, the internalizing moiety comprises a glutamine, rather than an asparagine, at a position corresponding to Kabat position 297.

In some embodiments, the antibody or antigen binding fragment comprises hybrid heavy chain constant regions, i.e., the antibody or antigen binding fragment comprise multiple heavy chain constant region domains selected from: a CH1 domain, a CH2 domain, a CH3 domain, and a CH4 domain; wherein at least one of the constant region domains in the antibody or antigen binding fragment is of a class or subclass of immunoglobulin distinct from the class or subclass of another domain in the antibody or antigen binding fragment. In some embodiments, at least one of the constant region domains in the antibody or antigen binding fragment is an IgG constant region domain, and at least one of the constant region domains in the antibody or antigen binding fragment is of a different immunoglobulin class, i.e., an IgA, IgD, IgE, or IgM constant region domain. In some embodiments, at least one of the constant region domains in the antibody or antigen binding fragment is an IgG1 constant region domain, and at least one of the constant region domains in the antibody or antigen binding fragment is of a different IgG subclass, i.e., an IgG2A, IgG2B, IgG3 or IgG4. Suitable constant regions may be human or from another species (e.g., murine). Humanized antibodies and antigen binding fragments of the disclosure are consider humanized regardless of whether and constant region sequence (heavy or light chain), if present, corresponds to that of a human immunoglobulin or corresponds to that of another species.

The cell penetrating ability of the humanized internalizing moieties or fragments or variants may be utilized to promote delivery of a heterologous agent. Humanized moieties derived from 3E10 are particularly well suited for this because of their demonstrated ability to effectively promote delivery to muscle, retinal, and neuronal cells. Thus, humanized internalizing moieties are especially useful for promoting effective delivery into cells in subjects, such as human patients or model organisms. In certain embodiments, antibodies and antigen binding fragments of the disclosure are useful as intermediates for further conjugation to a heterologous agent, such as a heterologous protein, peptide, polynucleotide, or small molecule. However, in other embodiments, the humanized internalizing moieties or fragments or variants are not utilized to deliver any heterologous agent.

Preparation of antibodies or fragments thereof (e.g., a single chain Fv fragment encoded by V_{H}-linker-V_{L} or V_{L}-linker-V_{H}) is well known in the art. In particular, methods of recombinant production of mAb 3E10 antibody fragments have been described in WO 2008/091911. Further, methods of generating scFv fragments of antibodies are well known in the art. When recombinantly producing an antibody or antibody fragment, a linker may be used. For example, typical surface amino acids in flexible protein regions include Gly, Asn and Ser. One exemplary linker is provided in SEQ ID NO: 5 or 16. Permutations of amino acid sequences containing Gly, Asn and Ser would be expected to satisfy the criteria (e.g., flexible with minimal hydrophobic or charged character) for a linker sequence. Another exemplary linker is of the formula (G₄S)n, wherein n is an integer from 1-10, such as 2, 3, or 4. Other near neutral amino acids, such as Thr and Ala, can also be used in the linker sequence.

In addition to linkers interconnecting portions of, for example, an scFv, the disclosure contemplates the use of additional linkers to, for example, interconnect the heterologous agent to the antibody portion of a conjugate or to interconnect the heterologous agent portion to the antibody portion of conjugate.

Preparation of antibodies may be accomplished by any number of well-known methods for generating monoclonal antibodies. These methods typically include the step of immunization of animals, typically mice, with a desired immunogen (e.g., a desired target molecule or fragment thereof). Once the mice have been immunized, and preferably boosted one or more times with the desired immunogen(s), monoclonal antibody-producing hybridomas may be prepared and screened according to well known methods (see, for example, Kuby, Janis, Immunology, Third Edition, pp. 131-139, W.H. Freeman & Co. (1997), for a general overview of monoclonal antibody production, that portion of which is incorporated herein by reference). Over the past several decades, antibody production has become extremely robust. In vitro methods that combine antibody recognition and phage display techniques allow one to amplify and select antibodies with very specific binding capabilities. See, for example, Holt, L. J. et al., “The Use of Recombinant Antibodies in Proteomics,” Current Opinion in Biotechnology, 2000, 11:445-449, incorporated herein by reference. These methods typically are much less cumbersome than preparation of hybridomas by traditional monoclonal antibody preparation methods. In one embodiment, phage display technology may be used to generate an internalizing moiety specific for a desired target molecule. An immune response to a selected immunogen is elicited in an animal (such as a mouse, rabbit, goat or other animal) and the response is boosted to expand the immunogen-specific B-cell population. Messenger RNA is isolated from those B-cells, or optionally a monoclonal or polyclonal hybridoma population. The mRNA is reverse-transcribed by known methods using either a poly-A primer or murine immunoglobulin-specific primer(s), typically specific to sequences adjacent to the desired V_{H} and V_{L} chains, to yield cDNA. The desired V_{H} and V_{L} chains are amplified by polymerase chain reaction (PCR) typically using V_{H} and V_{L} specific primer sets, and are ligated together, separated by a linker. V_{H} and V_{L} specific primer sets are commercially available, for instance from Stratagene, Inc. of La Jolla, Calif. Assembled V_{H}-linker-V_{L} product (encoding an scFv fragment) is selected for and amplified by PCR. Restriction sites are introduced into the ends of the V_{H}-linker-V_{L} product by PCR with primers including restriction sites and the scFv fragment is inserted into a suitable expression vector (typically a plasmid) for phage display. Other fragments, such as an Fab′ fragment, may be cloned into phage display vectors for surface expression on phage particles. The phage may be any phage, such as lambda, but typically is a filamentous phage, such as fd and M13, typically M13.

In certain embodiments, an antibody or antibody fragment is made recombinantly in a host cell. In other words, once the sequence of the antibody is known (for example, using the methods described above), the antibody can be made recombinantly using standard techniques.

In certain embodiments, the humanized internalizing moieties may be modified to make them more resistant to cleavage by proteases. For example, the stability of an internalizing moiety comprising a polypeptide may be increased by substituting one or more of the naturally occurring amino acids in the (L) configuration with D-amino acids. In various embodiments, at least 1%, 5%, 10%, 20%, 50%, 80%, 90% or 100% of the amino acid residues of internalizing moiety may be of the D configuration. The switch from L to D amino acids neutralizes the digestion capabilities of many of the ubiquitous peptidases found in the digestive tract. Alternatively, enhanced stability of an internalizing moiety comprising a peptide bond may be achieved by the introduction of modifications of the traditional peptide linkages. For example, the introduction of a cyclic ring within the polypeptide backbone may confer enhanced stability in order to circumvent the effect of many proteolytic enzymes known to digest polypeptides in the stomach or other digestive organs and in serum. In still other embodiments, enhanced stability of an internalizing moiety may be achieved by intercalating one or more dextrorotatory amino acids (such as, dextrorotatory phenylalanine or dextrorotatory tryptophan) between the amino acids of internalizing moiety. In exemplary embodiments, such modifications increase the protease resistance of an internalizing moiety without affecting the activity or specificity of the interaction with a desired target molecule.

A “Fab fragment” is comprised of one light chain and the C_{H}1 and variable regions of one heavy chain. Generally, the heavy chain of a Fab molecule cannot form a disulfide bond with another heavy chain molecule. A Fab may optionally include a portion of the hinge, such as the upper hinge.

A “Fab′ fragment” contains one light chain and one heavy chain that contains more of the constant region, between the C_{H}1 and C_{H}2 domains, such that an interchain disulfide bond can be formed between two heavy chains to form a F(ab′)₂ molecule.

A “F(ab′)₂ fragment” contains two light chains and two heavy chains containing a portion of the constant region between the CH1 and CH2 domains, such that an interchain disulfide bond is formed between two heavy chains.

Native antibodies are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains (CH). Each light chain has a variable domain at one end (VL) and a constant domain (CL) at its other end; the constant domain of the light chain is aligned with the first constant domain of the heavy chain, and the light chain variable domain is aligned with the variable domain of the heavy chain. Light chains are classified as either lambda chains or kappa chains based on the amino acid sequence of the light chain constant region. The variable domain of a kappa light chain may also be denoted herein as VK.

The antibodies of the disclosure include full-length or intact antibody, antibody fragments, native sequence antibody or amino acid variants, human, humanized (a form of chimeric antibodies), post-translationally modified, chimeric antibodies, immunoconjugates, and functional fragments thereof. The antibodies can be modified in the Fc region to provide desired effector functions or serum half-life.

Preparation of Antibodies

Naturally occurring antibody structural units typically comprise a tetramer. Each such tetramer typically is composed of two identical pairs of polypeptide chains, each pair having one full-length “light” chain (typically having a molecular weight of about 25 kDa) and one full-length “heavy” chain (typically having a molecular weight of about 50-70 kDa). The amino-terminal portion of each chain typically includes a variable region of about 100 to 110 or more amino acids that typically is responsible for antigen recognition. The carboxy-terminal portion of each chain typically defines a constant region responsible for effector function. Human light chains are typically classified as kappa and lambda light chains. Heavy chains are typically classified as mu, delta, gamma, alpha, or epsilon, and define the antibody's isotype as IgM, IgD, IgG, IgA, and IgE, respectively. IgG has several subclasses, including, but not limited to, IgG1, IgG2, IgG3, and IgG4. IgM has subclasses including, but not limited to, IgM1 and IgM2. IgA is similarly subdivided into subclasses including, but not limited to, IgA1 and IgA2. See, e.g., Fundamental Immunology, Ch. 7, 2.sup.nd ed., (Paul, W., ed.), 1989, Raven Press, N.Y. (incorporated by reference in its entirety for all purposes). The combination of the variable regions of each light chain/heavy chain pair typically forms the antigen-binding site. In some embodiments, antibodies or antigen binding fragments of the disclosure comprise the following constant domain scheme: IgG2a CH1-IgG1 hinge-IgG1 CH2-CH3. Other suitable combinations are also contemplated. In other embodiments, the antibody comprises a full-length antibody and the CH1, hinge, CH2, and CH3 is from the same constant domain subclass (e.g., IgG1). In some embodiments, the antibodies or antigen binding fragment comprises an antigen binding fragment comprising a portion of the constant domain of an immunoglobulin, for example, the following constant domain scheme: IgG2a CH1-IgG1 upper hinge. In some embodiments, the antibodies or antigen binding fragments of the disclosure comprise a kappa constant domain (e.g., SEQ ID NO: 9).

The variable regions of each of the heavy chains and light chains typically exhibit the same general structure comprising four relatively conserved framework regions (FR) joined by three hyper variable regions, also called complementarity determining regions or CDRs. The CDRs from the two chains of each pair typically are aligned by the framework regions, which alignment may enable binding to a specific target (e.g., antigen, DNA in the context of the present disclosure). From N-terminal to C-terminal, both light and heavy chain variable regions typically comprise the domains FR1, CDR1, FR2, CDR2, FR3, CDR3 and FR4. The assignment of amino acids to each domain (FR or CDR) is typically in accordance with the definitions of Kabat Sequences of Proteins of Immunological Interest (1987 and 1991, National Institutes of Health, Bethesda, Md.). In certain embodiments, the CDRs of a particular antibody, such as an antibody provided herein, are CDRs, as defined by this Kabat system (e.g., the CDRs being referred to for an antibody or antigen binding fragment are identified using the Kabat system). Similarly, in certain embodiments, particularly when the CDRs are defined or identified as by the Kabat system, the FR regions are also defined and/or identified using the Kabat system. However, alternative systems for identifying CDR and FR regions are also available, including the IMGT system (described herein). In certain embodiments, the CDRs of a particular antibody, such as an antibody provided herein, are CDRs as defined by the IMGT system (e.g., CDRs for an antibody or antigen binding fragment are identified using the IMGT system).

Antibodies became useful and of interest as pharmaceutical agents with the development of monoclonal antibodies. Monoclonal antibodies are produced using any method that produces antibody molecules by continuous cell lines in culture. Examples of suitable methods for preparing monoclonal antibodies include the hybridoma methods of Kohler et al. (1975, Nature 256:495-497) and the human B-cell hybridoma method (Kozbor, 1984, J. Immunol. 133:3001; and Brodeur et al., 1987, Monoclonal Antibody Production Techniques and Applications, (Marcel Dekker, Inc., New York), pp. 51-63). In many cases, hybridomas are used to generate an initial antibody of murine or rodent origin. That initial antibody may then be modified, such as using recombinant techniques to produce rodent variants, chimeric antibodies, humanized antibodies and the like. Other methods exist to produce an initial antibody, and such methods are known in the art. However, regardless of the method used to generate an initial antibody or even a variant of that initial antibody, any given antibody of non-human origin can then be modified to increase its humanness.

It can be advantageous to increase the humanness of a non-human antibody to make it more suitable for use in human subject and cells, whether for diagnostic, therapeutic, or research purposes. Antibodies may be modified for use as therapeutics. Examples of such antibodies (including antibody fragments) include chimeric, humanized, and fully human antibodies. Numerous methods exist in the art for the generation of chimeric, humanized and human antibodies. In the context of the present disclosure, an antibody is considered humanized if at least one of the VH domain or VL domain is humanized. Moreover, a VH or VL domain is humanized if the amino acid sequence of at least a portion of at least one FR regions has been modified, relative to a parent murine antibody, such that the amino acid sequence of that portion corresponds to that of a human antibody or a human consensus sequence. In certain embodiments, at least one, two, three, or four FR regions of the VH domain and/or at least one, two, three, or four FR regions of the VL domain have been modified (in whole or in part) so that their sequence is more closely related to a human sequence. For any of the foregoing in certain embodiments, a humanized antibody fragment may be provided in the context of a human or non-human light chain and/or heavy chain constant region (e.g., comprising a CL and one or more of a CH1, hinge, CH2, and/or CH3 domains). In certain embodiments, a humanized antibody or antigen binding fragment of the disclosure is provided in the context of human light and/or heavy chain constant domains, when present. Numerous examples of humanized light and heavy chain variable domains based on a 3E10 parent antibody are provided herein. Antibodies and antibody binding fragments combining any of the humanized light chain variable domains and/or heavy chain variable domains described herein are exemplary of antibodies and antigen binding fragments of the disclosure.

Once the nucleotide sequences encoding such antibodies have been determined, chimeric or humanized antibodies may be produced by recombinant methods. Nucleic acids encoding the antibodies are introduced into host cells and expressed using materials and procedures generally known in the art.

In certain embodiments, the antibodies or antigen binding fragments of the disclosure are of the IgG1, IgG2, or IgG4 isotype. In certain embodiments of the disclosure, the antibodies comprise a human kappa light chain and a human IgG1, IgG2, or IgG4 heavy chain. In certain embodiments, the antibodies of the disclosure have been cloned for expression in mammalian cells.

Regardless of when an antibody of the disclosure is a full-length antibody or an antigen binding fragment, antibodies and antigen binding fragments of the disclosure can be recombinantly expressed in cell lines. In these embodiments, sequences encoding particular antibodies can be used for transformation of a suitable host cell, such as a mammalian host cell or yeast host cell. According to these embodiments, transformation can be achieved using any known method for introducing polynucleotides into a host cell, including, for example packaging the polynucleotide in a virus (or into a viral vector) and transducing a host cell with the virus (or vector) or by transfection procedures known in the art. Generally, the transformation procedure used may depend upon the host to be transformed. Methods for introducing heterologous polynucleotides into mammalian cells are well known in the art and include, but are not limited to, dextran-mediated transfection, calcium phosphate precipitation, polybrene mediated transfection, protoplast fusion, electroporation, encapsulation of the polynucleotide(s) in liposomes, and direct microinjection of the DNA into nuclei.

According to certain embodiments of the disclosure, a nucleic acid molecule encoding the amino acid sequence of a heavy chain constant region (all or a portion), a heavy chain variable region of the disclosure, a light chain constant region, or a light chain variable region of the disclosure is inserted into an appropriate expression vector using standard ligation techniques. In a preferred embodiment, the heavy or light chain constant region is appended to the C-terminus of the appropriate variable region and is ligated into an expression vector. The vector is typically selected to be functional in the particular host cell employed (i.e., the vector is compatible with the host cell machinery such that amplification of the gene and/or expression of the gene can occur). For a review of expression vectors, see, Goeddel (ed.), 1990, Meth. Enzymol. Vol. 185, Academic Press. N.Y. In the context of antibody expression, both the heavy and light chain may be expressed from the same vector (e.g., from the same or different promoters present on the same vector) or the heavy and light chains may be expressed from different vectors. In certain embodiments, the heavy and light chains are expressed from different vectors which are transfected into the same host cell and co-expressed. Regardless of when the heavy and light chains are expressed in the same host cell from the same or a different vector, the chains can then associate to form an antibody (or antibody fragment, depending on the portions of the heavy and light chain being expressed).

In some embodiments, an antibody or antigen binding fragment of the disclosure is not conjugated to a heterologous agent. In other embodiments, an antibody or antigen binding fragment of the disclosure is conjugated to a heterologous agent. In certain embodiments, the heterologous agent is a protein or peptide. That protein or peptide may be expressed as an inframe, co-translation fusion protein with, for example, the heavy chain, and expressed as described herein. Chemical conjugation is also possible. Conjugated as described in detail herein and unless otherwise specified, refers to scenarios where any of the antibody or antigen binding portions of the disclosure are associated with or interconnected with the heterologous agent, regardless of the interconnection (e.g., the interconnection/association may comprise a chemical conjugation, covalent bond, di-sulfide bond, etc. or combinations thereof). In certain embodiments, at least a portion of the interconnection is via a covalent bond, such as the forming of a fusion protein between a heavy chain of the antibody of the disclosure and the heterologous agent (which may further associate with a light chain of the antibody of the disclosure). Accordingly, the disclosure provides such conjugates and pharmaceutical compositions comprising such conjugates. A conjugate is a molecule comprising an antibody or antigen binding portion of the disclosure associate with a heterologous agent. Similarly, antibodies or antigen binding fragments of the disclosure may further comprise a heterologous agent. Conjugates along molecules where the two portions are associated or interconnected (e.g., the interconnection may comprise a chemical conjugation, covalent bond, di-sulfide bond, etc. or combinations thereof). In certain embodiments, at least a portion of the interconnection is via a covalent bond, such as the forming of a fusion protein between a heavy chain of an antibody of the disclosure and the heterologous agent (which may further associate with a light chain of the antibody or antibody fragment of the disclosure).

Typically, expression vectors used in any of the host cells will contain sequences for plasmid maintenance and for cloning and expression of exogenous nucleotide sequences. Such sequences, collectively referred to as “flanking sequences” in certain embodiments will typically include one or more of the following nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. These portions of vectors are well known, and there are numerous generally available vectors that can be selected and used for the expression of proteins. One can readily select vectors based on the desired host cell and application.

An origin of replication is typically a part of those prokaryotic expression vectors purchased commercially, and the origin aids in the amplification of the vector in a host cell. If the vector of choice does not contain an origin of replication site, one may be chemically synthesized based on a known sequence, and ligated into the vector. For example, the origin of replication from the plasmid pBR322 (New England Biolabs, Beverly, Mass.) is suitable for most gram-negative bacteria and various viral origins (e.g., SV40, polyoma, adenovirus, vesicular stomatitus virus (VSV), or papillomaviruses such as HPV or BPV) are useful for cloning vectors in mammalian cells. Generally, the origin of replication component is not needed for mammalian expression vectors (for example, the SV40 origin is often used only because it also contains the virus early promoter).

The expression and cloning vectors of the disclosure will typically contain a promoter that is recognized by the host organism and operably linked to the molecule encoding heavy and/or light chain. Promoters are untranscribed sequences located upstream (i.e., 5′) to the start codon of a structural gene (generally within about 100 to 1000 bp) that control the transcription of the structural gene. Promoters are conventionally grouped into one of two classes: inducible promoters and constitutive promoters. Inducible promoters initiate increased levels of transcription from DNA under their control in response to some change in culture conditions, such as the presence or absence of a nutrient or a change in temperature. Constitutive promoters, on the other hand, initiate continual gene product production; that is, there is little or no control over gene expression. A large number of promoters, recognized by a variety of potential host cells, are well known. A suitable promoter is operably linked to the DNA encoding the heavy chain or light chain comprising an antibody or antigen binding fragment of the disclosure. In certain embodiments, the same promoter is used for both the heavy and light chain. In other embodiments, different promoters (present on the same or different vectors) are used for each.

Suitable promoters for use with yeast hosts are also well known in the art. Yeast enhancers are advantageously used with yeast promoters. Suitable promoters for use with mammalian host cells are well known and include, but are not limited to, those obtained from the genomes of viruses such as polyoma virus, fowlpox virus, adenovirus (such as Adenovirus 2), bovine papilloma virus, avian sarcoma virus, cytomegalovirus, retroviruses, hepatitis-B virus and most preferably Simian Virus 40 (SV40). Other suitable mammalian promoters include heterologous mammalian promoters, for example, heat-shock promoters and the actin promoter.

Additional promoters which may be of interest include, but are not limited to: the SV40 early promoter region (Bernoist and Chambon, 1981, Nature 290:304-10); the CMV promoter; the promoter contained in the 3′ long terminal repeat of Rous sarcoma virus (Yamamoto et al., 1980, Cell 22:787-97); the herpes thymidine kinase promoter (Wagner et al., 1981, Proc. Natl. Acad. Sci. USA 78:1444-45); the regulatory sequences of the metallothionine gene (Brinster et al., 1982, Nature 296:39-42); prokaryotic expression vectors such as the beta-lactamase promoter (Villa-Kamaroff et al., 1978, Proc. Natl. Acad. Sci. USA 75:3727-31); or the tac promoter (DeBoer et al., 1983, Proc. Natl. Acad. Sci. USA 80:21-25). Also of interest are the following animal transcriptional control regions, which exhibit tissue specificity and have been utilized in transgenic animals: the elastase I gene control region that is active in pancreatic acinar cells (Swift et al., 1984, Cell 38:639-46; Ornitz et al., 1986, Cold Spring Harbor Symp. Quant. Biol. 50:399-409 (1986); MacDonald, 1987, Hepatology 7:425-515); the insulin gene control region that is active in pancreatic beta cells (Hanahan, 1985, Nature 315:115-22); the immunoglobulin gene control region that is active in lymphoid cells (Grosschedl et al., 1984, Cell 38:647-58; Adames et al., 1985, Nature 318:533-38; Alexander et al., 1987, Mol. Cell. Biol. 7:1436-44); the mouse mammary tumor virus control region that is active in testicular, breast, lymphoid and mast cells (Leder et al., 1986, Cell 45:485-95); the albumin gene control region that is active in liver (Pinkert et al., 1987, Genes and Devel. 1:268-76); the alpha-feto-protein gene control region that is active in liver (Krumlauf et al., 1985, Mol. Cell. Biol. 5:1639-48; Hammer et al., 1987, Science 235:53-58); the alpha 1-antitrypsin gene control region that is active in liver (Kelsey et al., 1987, Genes and Devel. 1:161-71); the beta-globin gene control region that is active in myeloid cells (Mogram et al., 1985, Nature 315:338-40; Kollias et al., 1986, Cell 46:89-94); the myelin basic protein gene control region that is active in oligodendrocyte cells in the brain (Readhead et al., 1987, Cell 48:703-12); the myosin light chain-2 gene control region that is active in skeletal muscle (Sani, 1985, Nature 314:283-86); and the gonadotropic releasing hormone gene control region that is active in the hypothalamus (Mason et al., 1986, Science 234:1372-78).

The vector may also include an enhancer sequence to increase transcription of DNA encoding light chain or heavy chain.

Expression vectors of the disclosure may be constructed from a starting vector such as a commercially available vector. Such vectors may or may not contain all of the desired flanking sequences. Where one or more of the flanking sequences described herein are not already present in the vector, they may be individually obtained and ligated into the vector. Methods used for obtaining each of the flanking sequences are well known to one skilled in the art.

After the vector has been constructed and a nucleic acid molecule encoding light chain or heavy chain or light chain and heavy chain comprising an antibody or antigen binding fragment of the disclosure has been inserted into the proper site of the vector, the completed vector may be inserted into a suitable host cell for amplification and/or polypeptide expression. The transformation of an expression vector into a selected host cell may be accomplished by well known methods including transfection, infection, calcium phosphate co-precipitation, electroporation, microinjection, lipofection, DEAE-dextran mediated transfection, or other known techniques. The method selected will in part be a function of the type of host cell to be used. These methods and other suitable methods are well known to the skilled artisan, and are set forth, for example, in Sambrook et al., supra.

The host cell, when cultured under appropriate conditions, synthesizes the antibody or antigen binding fragment of the disclosure that can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity (such as glycosylation or phosphorylation) and ease of folding into a biologically active molecule.

Mammalian cell lines available as hosts for expression are well known in the art and include, but are not limited to, many immortalized cell lines available from the American Type Culture Collection (A.T.C.C.), including but not limited to Chinese hamster ovary (CHO) cells, HeLa cells, baby hamster kidney (BHK) cells, monkey kidney cells (COS), human hepatocellular carcinoma cells (e.g., Hep G2), and a number of other cell lines. In another embodiment, one may select a cell line from the B cell lineage that does not make its own antibody but has a capacity to make and secrete a heterologous antibody (e.g., mouse myeloma cell lines NS0 and SP2/0). In other embodiments, a cell other than a mammalian cell is used, such as a yeast cell line (e.g., Pichia).

In certain embodiments, the cell line stably expresses an antibody or antigen binding fragment of the disclosure. In other embodiments, the cells transiently express an antibody or antigen binding fragment of the disclosure.

In certain embodiments is provided antibodies of the disclosure (including antigen binding fragments) that are substantially purified/isolated. Numerous methods, filters, and devices for substantially purifying antibodies grown in recombinant cell culture are available.

Antibody fragments can also be made by enzymatic digestion of a full-length antibody.

In certain embodiments, the antibodies or antigen binding fragments of the disclosure, whether provided alone or as conjugates with a heterologous agent, are detectably labeled. In certain embodiments, the detectable label is itself an example of a heterologous agent. Methods for conjugation to a substance, such as a detectable label, are well known in the art. In one embodiment, the attached substance is a detectable label (also referred to herein as a reporter molecule). Suitable substances for attachment to include, but are not limited to, a fluorophore, a chromophore, a dye, a radioisotope, and combinations thereof. Methods for conjugation or covalently attaching another substance to an antibody are well known in the art.

The terms “label” or “labeled” refers to incorporation of a detectable marker, e.g., by incorporation of a radiolabeled amino acid or attachment to a polypeptide of biotin moieties that can be detected by marked avidin (e.g., streptavidin preferably comprising a detectable marker such as a fluorescent marker, a chemiluminescent marker or an enzymatic activity that can be detected by optical or colorimetric methods). Various methods of labeling polypeptides and glycoproteins are known in the art and may be used advantageously in the methods disclosed herein. Examples of labels for polypeptides include, but are not limited to, the following: radioisotopes or radionuclides (e.g., ³H, ¹⁴C, ¹⁵N, ³⁵S, ⁹⁰Y, ⁹⁹mTc, ¹¹¹In, ¹²⁵I, ¹³¹I). In certain embodiments, the label is a radioactive isotope. Examples of suitable radioactive materials include, but are not limited to, iodine (¹²³I, ¹²⁵I, ¹³¹I), carbon (¹⁴C), sulfur (³⁵S), tritium (³H), indium (¹¹¹In, ¹¹²In, ¹¹³mIn, ¹¹⁵mIn), technetium (⁹⁹Tc, ⁹⁹mTc), thallium (²⁰¹Ti), gallium (⁸⁸Ga, ⁶⁷Ga), palladium (¹⁰³Pd), molybdenum (⁹⁹Mo), xenon (¹³⁵Xe), fluorine (¹⁸F), ¹⁵⁵SM, ¹⁷⁷Lu, ¹⁵⁹Gd, ¹⁴⁹Pm, ¹⁴⁰La, ¹⁷⁵Yb, ¹⁶⁶Ho, ⁹⁰Y ⁴⁷Sc, ¹⁸⁶Re, ¹⁸⁸Re, ¹⁴²Pr, ¹⁰⁵Rh and ⁹⁷Ru.

Further examples of labels include fluorescent labels (e.g., fluoroscein isothiocyanate (FITC), rhodamine, or lanthanide phosphors), enzymatic labels (e.g., horseradish peroxidase, β-galactosidase, luciferase, alkaline phosphatase), chemiluminescent labels, hapten labels such as biotinyl groups, and predetermined polypeptide epitopes recognized by a secondary reporter (e.g., leucine zipper pair sequences, binding sites for secondary antibodies, metal binding domains, epitope tags). In certain embodiments, labels are attached by spacer arms of various lengths to reduce potential steric hindrance.

When present, regardless of the particular label, one of skill can select an appropriate label to facilitate purification, diagnostic, or research use. In other embodiments, the heterologous agent is a therapeutic molecule and either does not include a detectable label and/or epitope tag, or includes a therapeutic molecule in addition to the detectable label and/or epitope tag.

“Humanized” refers to an immunoglobulin such as an antibody, wherein the amino acids directly involved in antigen binding, the so-called complementary determining regions (CDR), of the heavy and light chains are not necessarily of human origin, while at least a portion of the rest of the variable domain (e.g., one or more of FR1, FR2, FR3, FR4) of one or both chains of the immunoglobulin molecule, the so-called framework regions of the variable heavy and/or light chains, and, if present, optionally the constant regions of the heavy and light chains are modified so that their amino acid sequence more closely correspond to human sequences.

A “humanized antibody” as used herein in the case of a two or greater chain antibody is one where at least one chain is humanized. A humanized antibody chain has a variable region where one or more of the framework regions are human or contain alterations, relative to a murine parent, so that one or more framework regions are more human than a murine parent. A humanized antibody which is a single chain is one where the chain has a variable region where one or more of the framework regions are human or contain alterations, relative to a murine parent, so that one or more framework regions are more human. The non-human portions of the variable region of the humanized antibody chain or antigen-binding fragment is derived from a non-human source, particularly a non-human antibody, typically of rodent origin. The non-human contribution to the humanized antibody is typically provided in the form of at least one CDR region which is interspersed among framework regions derived from one (or more) human immunoglobulin(s). In addition, framework support residues may be altered to preserve binding affinity. Thus, as is understood in the art, an entire framework region or all of the framework regions on a particular chain need not contain residues corresponding to a human antibody in order for the antibody to be considered humanized.

A “humanized antibody” may further comprise constant regions (e.g., at least one constant region or portion thereof, in the case of a light chain, and in some embodiments three constant regions in the case of a heavy chain). The constant regions of a humanized antibody, if present, typically are human in origin.

In some embodiments, a humanized antibody is generated by first subjecting a murine 3E10 light or heavy chain antibody sequence (e.g., the murine 3E10 antibody light and heavy chain amino acid sequences of SEQ ID NO: 18 and 17, respectively) to a sequence database search (e.g., BLAST) in order to identify the top closest human immunoglobulin kappa or heavy chain homologues in sequence similarity (e.g., the top 1, 2, 3, 4, 5, 6, 7, 8, 9 or 10 closest immunoglobulin kappa or heavy chain homologues). The top closest human immunoglobulin kappa or heavy chain homologues are considered candidates for kappa or heavy chain CDR grafting. In some embodiments, sequence alignment tools, such as Vector NTI sequence alignment tools, are then used to analyze the chimeric amino acid sequences consisting of the CDRs from the 3E10 kappa or heavy chain and the framework regions of any one of the top human immunoglobulin kappa or heavy chain homologues.

In general, as used herein, humanized antibodies comprise one or two variable domains in which all or part of the CDR regions correspond to parts derived from the non-human parent sequence and in which all or part of the FR regions are derived from a human immunoglobulin sequence. The humanized antibody can then, optionally, comprise at least one portion of a constant region of immunoglobulin (Fc), in particular that of a selected reference human immunoglobulin.

In some embodiments, the antibodies and antigen binding fragments of the disclosure (e.g., an antibody or antigen binding fragment, such as a humanized antibody or antigen binding fragment) comprises one or more of the CDRs of the 3E10 antibody. In certain embodiments, the antibodies and antigen binding fragments comprise one or more of the CDRs of a 3E10 antibody comprising a V_{H} domain comprising the amino acid sequence set forth in SEQ ID NO: 17 and a V_{L} domain comprising the amino acid sequence set forth in SEQ ID NO: 18. Either or both of the Kabat or IMGT CDRs may be used to refer to or describe an antibody. CDRs of the 3E10 antibody or an antibody of the disclosure may be determined using any of the CDR identification schemes available in the art, and such scheme may be used to describe the antibody. For example, in some embodiments, the CDRs are defined according to the Kabat definition as set forth in Kabat et al. Sequences of Proteins of Immunological Interest, 5th Ed. Public Health Service, National Institutes of Health, Bethesda, Md. (1991). In other embodiments, the CDRs are defined according to Chothia et al., 1987, J Mol Biol. 196: 901-917 and Chothia et al., 1989, Nature. 342:877-883. In other embodiments, the CDRs are defined according to the international ImMunoGeneTics database (IMGT) as set forth in LeFranc et al., 2003, Development and Comparative Immunology, 27: 55-77. In other embodiments, the CDRs of the 3E10 antibody are defined according to Honegger A, Pluckthun A., 2001, J Mol Biol., 309:657-670. In some embodiments, the CDRs are defined according to any of the CDR identification schemes discussed in Kunik et al., 2012, PLoS Comput Biol. 8(2): e1002388. In certain embodiments, antibodies and antigen binding fragments of the disclosure comprise one or more differences in the Kabat CDRs as compared to the murine, parent antibody. For example, in certain embodiments, the antibodies and antigen binding fragments of the disclosure differ at VH CDR2 and/or VL CDR2 and, optionally, at VL CDR1 in comparison to the murine, parent antibody. However, in certain embodiments, such antibodies share the IMGT CDRs of the murine, parent antibody.

Herein, the amino acid positions of residues in the VH and VL domains are referred to by linear sequence relative to, for example, SEQ ID NO: 17 or 18. Thus, the sequence of the VH and/or VL of an antibody or antigen binding fragment of the disclosure can be described relative to the corresponding amino acid position(s) of SEQ ID NO: 17 or 18. For example, a VH or VL domain may include an alteration at a particular amino acid position, and that position may correspond to a particular position in SEQ ID NO: 17 or 18.

However, the CDR identification scheme also provides numbering systems that may be used to facilitate comparisons between antibodies. Although not specifically used herein, one of skill in the art can readily use the available numbering scheme to refer to the CDRs described herein using a uniform numbering system, rather than by referring to the linear sequence. In certain embodiments, to number residues of an antibody for the purpose of identifying CDRs according to any of the CDR identification schemes known in the art, one may align the antibody at regions of homology of the sequence of the antibody with a “standard” numbered sequence known in the art for the elected CDR identification scheme. Maximal alignment of framework residues frequently requires the insertion of “spacer” residues in the numbering system, to be used for the Fv region. In addition, the identity of certain individual residues at any given site number may vary from antibody chain to antibody chain due to interspecies or allelic divergence. These uniform schemes for numbering residues are not expressly used herein, but can be readily used based on the disclosed sequences and identified CDRs.

In certain embodiments, the antibodies and antigen binding fragments of the disclosure (e.g., a humanized antibody or antigen binding fragment of the disclosure) comprises Kabat CDRs. In some embodiments, the antibodies and antigen binding fragments comprise a V_{H} CDR1 that corresponds to amino acid residues 31-35 of SEQ ID NO: 17, a VH CDR2 that corresponds to amino acid residues 50-66 of SEQ ID NO: 17, and/or a V_{H} CDR3 that corresponds to amino acid residues 99-105 of SEQ ID NO: 17. We note that this numbering of amino acid residues is with reference to the linear amino acid sequence of SEQ ID NO: 17. One of skill in the art can readily use the Kabat system to identify these residues using Kabat numbering. In certain embodiments, the antibodies and antigen binding fragments comprise a V_{L} CDR1 that corresponds to amino acid residues 24-38 of SEQ ID NO: 18, a V_{L} CDR2 that corresponds to amino acid residues 54-60 of SEQ ID NO: 18, and/or a V_{L} CDR3 that corresponds to amino acid residues 93-101 of SEQ ID NO: 18. We note that this numbering of amino acid residues is with reference to the linear amino acid sequence of SEQ ID NO: 18. One of skill in the art can readily use the Kabat system to identify these residues using Kabat numbering.

In certain embodiments, the antibodies and antigen binding fragments of the disclosure comprise CDRs that are defined using the IMGT system. In some embodiments, the antibodies and antigen binding fragments comprise a V_{H} CDR1 that corresponds to amino acid residues 26-33 of SEQ ID NO: 17, a V_{H} CDR2 that corresponds to amino acid residues 51-58 of SEQ ID NO: 17, and/or a V_{H} CDR3 that corresponds to amino acid residues 97-105 of SEQ ID NO: 17. We note that this numbering of amino acid residues is with reference to the linear amino acid sequence of SEQ ID NO: 17. In certain embodiments, the antibodies and antigen binding fragments comprise a V_{L} CDR1 that corresponds to amino acid residues 27-36 of SEQ ID NO: 18, a V_{L} CDR2 that corresponds to amino acid residues 54-56 of SEQ ID NO: 18, and/or a V_{L} CDR3 that corresponds to amino acid residues 93-101 of SEQ ID NO: 18. We note that this numbering of amino acid residues is with reference to the linear amino acid sequence of SEQ ID NO: 18. In certain embodiments, an antibody or antigen binding fragment of the disclosure comprises all 6 of the foregoing CDRs. In certain embodiments, the antibody or antigen binding fragment comprises 4 of the foregoing CDRs, and a VH CDR2 as set forth in SEQ ID NO: 25 and a VL CDR 2 as set forth in SEQ ID NO: 27.

In certain embodiments, the antibodies and antigen binding fragments of the disclosure comprise at least 1, 2, 3, 4, or 5 of the CDRs of 3E10 as determined using the Kabat CDR identification scheme (e.g., the CDRs set forth in SEQ ID NOs: 19-24). In certain embodiments, the antibody or antigen binding fragment further comprises a VH CDR2 as set forth in SEQ ID NO: 25 and/or a VL CDR2 as set forth in SEQ ID NO: 27 and/or a VL CDR1 as set forth in SEQ ID NO: 26. In certain embodiments, the antibodies and antigen binding fragments comprise at least 1, 2, 3, 4 or 5 of the CDRS of 3E10 as determined using the IMGT identification scheme (e.g., the CDRs set forth in SEQ ID NOs: 10-15). In certain embodiments, the antibodies and antigen binding fragments comprise all six CDRs of 3E10 as determined using the Kabat CDR identification scheme (e.g., comprises SEQ ID NOs 19-24). In other embodiments, the antibodies and antigen binding fragments comprise all six CDRS of 3E10 as determined using the IMGT identification scheme (e.g., which are set forth as SEQ ID NOs: 10-15). For any of the foregoing, in certain embodiments, the antibodies and antigen binding fragments is an antibody that binds the same epitope (e.g., the same target, such as DNA) as 3E10 and/or the internalizing moiety competes with 3E10 for binding to antigen (e.g., DNA). Exemplary antibodies and antigen binding fragments can transit cells via ENT2 and/or ENT3. In certain embodiments, antibodies or antigen binding fragments of the disclosure comprise 6 of the foregoing CDRs, but include 1, 2 3, or 4 amino acid substitutions in one or more CDRs. For example, the antibodies or antigen binding fragments comprise 3 CDR substitutions: one substitution in each of three CDRs.

In certain embodiments, antibodies or antigen binding fragments of the disclosure (e.g., a humanized antibody or antigen binding fragment of the disclosure) comprise an amino acid sequence having at least one, two, three, four, or five amino acid alterations in one or more CDRs using IMGT numbering (e.g., in one or more CDRs having the amino acid sequence of any one of SEQ ID NOs: 10-15, such as having 1-2, 1-3, 1-4, or 1-5 alternations) or Kabat numbering (e.g., in one or more CDRs having the amino acid sequence of any one of SEQ ID NOs: 19-24, such as having 1-2, 1-3, 1-4, or 1-5 alterations). In certain embodiments, antibodies or antigen binding fragments of the disclosure (e.g., a humanized antibody or antigen binding fragment of the disclosure) comprise an amino acid sequence having at least one, two, three, four, or five amino acid alterations in one or more CDRs using Kabat numbering (e.g., in one or more CDRs having the amino acid sequence of any one of SEQ ID NOs: 19-24, such as have 2, 3, 4, or 5 alterations) In some embodiments, antibodies or antigen binding fragments of the disclosure comprise a V_{L} domain comprising one or more of the following amino acid alterations: M37L, H38A or E59Q, as compared with and numbered with respect to the linear amino acid sequence of SEQ ID NO: 18. In some embodiments, any of the antibodies or antigen binding fragments disclosed herein comprise a V_{H} domain comprising a T63S alteration, as compared with and numbered with respect to the linear amino acid sequence of SEQ ID NO: 17. In some embodiments, antibodies or antigen binding fragments of the disclosure comprise a V_{L} domain comprising an E59Q alteration as compared with and numbered with respect to the linear amino acid sequence of SEQ ID NO: 18, and a V_{H} domain comprising a T63S alteration as compared with and numbered with respect to the linear amino acid sequence of SEQ ID NO: 17.

Without wishing to be bound by theory, one of the surprising findings of the present disclosure is the ability to generate antibodies and antigen-binding fragments that have improved DNA binding activity versus murine 3E10, and further include an amino acid alteration (here, a substitution) in certain Kabat CDRs. Moreover, in certain embodiments, these improved antibodies having CDR substitutions are, in certain embodiments, also humanized.

In certain embodiments, an internalizing moiety of the disclosure, such as an antibody or antibody fragment described herein, binds a given DNA substrate with higher affinity as compared to an antibody or scFv or Fv having the VH and VL of the antibody produced by the hybridoma deposited with the ATCC under ATCC accession number PTA-2439. In certain embodiments, an internalizing moiety for use in the methods of the present disclosure is not an antibody or antibody fragment having the VH and VL of the antibody produced by the hybridoma deposited with the ATCC under ATCC accession number PTA-2439. In some embodiments, an internalizing moiety for use in the methods of the present disclosure is not a murine antibody or antibody fragment.

In certain embodiments, the antibodies and antigen binding fragments of the disclosure comprise a variable heavy chain domain comprising at least one CDR different from the corresponding CDR set forth in SEQ ID NO: 17, as determined using the Kabat CDR identification scheme. In some embodiments, the at least one different CDR is V_{H} CDR2 as set forth in SEQ ID NO: 25.

In certain embodiments, the antibodies and antigen binding fragments of the disclosure comprise a variable light chain domain comprising at least one CDR different from the corresponding CDR set forth in SEQ ID NO: 18, as determined using the Kabat CDR identification scheme. In some embodiments, the at least one different CDR is a V_{L} CDR1 as set forth in SEQ ID NO: 26. In some embodiments, the at least one different CDR is a V_{L} CDR2 as set forth in SEQ ID NO: 27.

Where the acceptor is derived from a human immunoglobulin, one may optionally select a human framework sequence that is selected based on its homology to the donor framework sequence by aligning the donor framework sequence with various human framework sequences in a collection of human framework sequences, and select the most homologous framework sequence as the acceptor. The acceptor human framework may be from or derived from human antibody germline sequences available in public databases. Regardless of the specific methodologies used to generate a humanized antibody or antibody fragment, the antibody must be evaluated to make sure that it (i) retains the desired function of the parent, murine antibody (or optionally has enhanced function); (ii) does not have deleterious properties that make it difficult to make or use; and preferably (iii) possesses one or more advantageous properties in comparison to the murine, parent antibody. Whether and to what extent any or all of these occur for any specific humanized antibody is unpredictable and uncertain. This is particularly true where substitutions are also introduced into the CDRs. Moreover, amongst a panel of humanized antibodies or antibody fragments, some may not have the required activity and one or more antibodies that do have the required activity may have advantageous properties in comparison to other humanized antibodies. This too is unpredictable and uncertain.

In certain embodiments, the antibodies or antigen-binding fragments of the disclosure comprise a light chain variable (VL) domain and a heavy chain variable (VH) domain; wherein the VL domain is humanized and comprises:

a VH CDR1 having the amino acid sequence of SEQ ID NO: 10;

a VH CDR2 having the amino acid sequence of SEQ ID NO: 11; and

a VH CDR3 having the amino acid sequence of SEQ ID NO: 12; which CDRs are in accordance with the IMGT system and the VH domain is humanized and comprises:

a VL CDR1 having the amino acid sequence of SEQ ID NO: 13;

a VL CDR2 having the amino acid sequence of SEQ ID NO: 14; and

a VL CDR3 having the amino acid sequence of SEQ ID NO: 15; which CDRs are in accordance with the IMGT system, and wherein the antibody or antigen-binding fragment has increased DNA binding and/or cell penetration, relative to that of a murine 3E10 antibody comprising a light chain variable (VL) domain having the amino acid sequence of SEQ ID NO: 18 and a heavy chain variable (VH) domain having the amino acid sequence of SEQ ID NO: 17.

In certain embodiments the antibodies or antigen-binding fragments of the disclosure comprise a light chain variable (VL) domain and a heavy chain variable (VH) domain; wherein the VH domain comprises:

a VH CDR1 having the amino acid sequence of SEQ ID NO: 19;

a VH CDR2 having the amino acid sequence of SEQ ID NO: 25; and

a VH CDR3 having the amino acid sequence of SEQ ID NO: 21,

which CDRs are according to the Kabat system;
and the VL comprises:

a VL CDR1 having the amino acid sequence of SEQ ID NO: 26;

a VL CDR2 having the amino acid sequence of SEQ ID NO: 27; and

a VL CDR3 having the amino acid sequence of SEQ ID NO: 24,

which CDRs are according to the Kabat system;
wherein the antibody or antigen-binding fragment binds DNA.

In certain embodiments the antibodies or antigen-binding fragments of the disclosure comprise a light chain variable (VL) domain and a heavy chain variable (VH) domain; wherein the VH domain comprises:

a VH CDR1 having the amino acid sequence of SEQ ID NO: 19;

a VH CDR2 having the amino acid sequence of SEQ ID NO: 25; and

a VH CDR3 having the amino acid sequence of SEQ ID NO: 21,

which CDRs are according to Kabat;
and the VL comprises:

a VL CDR1 having the amino acid sequence of SEQ ID NO: 22;

a VL CDR2 having the amino acid sequence of SEQ ID NO: 27; and

a VL CDR3 having the amino acid sequence of SEQ ID NO: 24,

which CDRs are according to Kabat;
wherein the antibody or antigen-binding fragment binds DNA.

In certain embodiments, antibodies or antigen binding fragments of the disclosure penetrate cells (e.g., can transit the plasma membrane and enter into cells, such as cells expressing ENT2).

In some embodiments, the VH domain is humanized. In some embodiments, the VL domain is humanized.

In certain embodiments the antibodies or antigen-binding fragments of the disclosure comprise a V_{L} domain that comprises the amino acid sequence set forth in SEQ ID NO: 4, or an amino acid sequence that differs from SEQ ID NO: 4 by the presence of a total of 1, 2, 3, 4, 5, or 6 amino acid substitutions, insertions and/or deletions in the framework regions, as defined by the IMGT system, relative to SEQ ID NO: 4. In some embodiments, the VL domain comprises the amino acid sequence set forth in SEQ ID NO: 4.

In certain embodiments the antibodies or antigen-binding fragments of the disclosure comprise a V_{H} domain that comprises the amino acid sequence set forth in SEQ ID NO: 3, or an amino acid sequence that differs from SEQ ID NO: 3 by the presence of a total of 1, 2, 3, 4, 5, or 6 amino acid substitutions, insertions and/or deletions in the framework regions, as defined by the IMGT system, relative to SEQ ID NO: 3. In some embodiments, the VH domain comprises the amino acid sequence set forth in SEQ ID NO: 3.

In certain embodiments the antibodies or antigen-binding fragments of the disclosure comprise a light chain variable (V_{L}) domain and a heavy chain variable (V_{H}) domain; wherein the V_{L} domain is humanized and comprises the amino acid sequence set forth in SEQ ID NO: 4; wherein the V_{H} domain comprises three CDRs of the amino acid sequence set forth in SEQ ID NO: 17, wherein the antibody or antigen-binding fragment binds DNA and/or penetrates cells.

In certain embodiments the antibodies or antigen-binding fragments of the disclosure comprise a light chain variable (V_{L}) domain and a heavy chain variable (V_{H}) domain; wherein the V_{H} domain is humanized and comprises the amino acid sequence set forth in SEQ ID NO: 3; wherein the V_{L} domain comprises three CDRs of the amino acid sequence set forth in SEQ ID NO: 18, wherein the antibody or antigen-binding fragment binds DNA and/or penetrates cells.

In certain embodiments the antibodies or antigen-binding fragments of the disclosure comprise a light chain variable (V_{L}) domain and a heavy chain variable (V_{H}) domain; wherein the V_{H} domain is humanized and comprises the amino acid sequence set forth in SEQ ID NO: 3; wherein the V_{L} domain comprises three CDRs of the amino acid sequence set forth in SEQ ID NO: 4, wherein the antibody or antigen-binding fragment binds DNA and/or penetrates cells.

In certain embodiments, the V_{H} domain of the antibodies or antigen-binding fragments described herein comprise:

a VH CDR1 having the amino acid sequence of SEQ ID NO: 10;

a VH CDR2 having the amino acid sequence of SEQ ID NO: 11; and

a VH CDR3 having the amino acid sequence of SEQ ID NO: 12.

In certain embodiments, the V_{L} domain of the antibodies or antigen-binding fragments described herein comprise:

a VL CDR1 having the amino acid sequence of SEQ ID NO: 13;

a VL CDR2 having the amino acid sequence of SEQ ID NO: 14; and

a VL CDR3 having the amino acid sequence of SEQ ID NO: 15.

In some embodiments, the antibodies or antigen-binding fragments disclosed herein comprise a light chain variable (V_{L}) domain and a heavy chain variable (V_{H}) domain; wherein the V_{L} domain comprises the amino acid sequence set forth in SEQ ID NO: 4; wherein the V_{H} domain comprises three CDRs of the amino acid sequence set forth in SEQ ID NO: 17, wherein the antibody or antigen-binding fragment binds DNA and/or penetrates cells. In some embodiments, the antibodies or antigen-binding fragments disclosed herein comprise a light chain variable (V_{L}) domain and a heavy chain variable (V_{H}) domain; wherein the V_{H} domain comprises the amino acid sequence set forth in SEQ ID NO: 3; wherein the V_{L} domain comprises three CDRs of the amino acid sequence set forth in SEQ ID NO: 18, wherein the antibody or antigen-binding fragment binds DNA and/or penetrates cells.

In some embodiments, an antibody or antigen-binding fragment of the disclosure comprises: a) a humanized V_{H} domain that comprises the amino acid sequence of SEQ ID NO: 3, and b) a V_{L} domain that comprises the amino acid sequence of SEQ ID NO: 18. In some embodiments, an antibody or antigen-binding fragment of the disclosure comprises: a) a humanized V_{H} domain that comprises the amino acid sequence of SEQ ID NO: 3, and b) a humanized V_{L} domain that comprises the amino acid sequence of SEQ ID NO: 4. In some embodiments, an antibody or antigen-binding fragment of the disclosure comprises: a) a V_{H} domain that comprises the amino acid sequence of SEQ ID NO: 17, and b) a humanized V_{L} domain that comprises the amino acid sequence of SEQ ID NO: 4.

In some embodiments, an antibody or antigen-binding fragment of the disclosure includes a signal sequence. In some embodiments, the signal sequence is conjugated to the N-terminal portion of any of the V_{L} sequences disclosed herein (e.g., SEQ ID NO: 4). In some embodiments, the signal sequence is conjugated to the N-terminal portion of any of the V_{H} sequences disclosed herein (e.g., SEQ ID NO: 3). It is understood that, when a signal sequence is included for expression of an antibody or antibody fragment, that signal sequence is generally cleaved and not present in the finished polypeptide (e.g., the signal sequence is generally cleaved and present only transiently during protein production).

In some embodiments, the V_{H} domain of any of the antibodies or antigen-binding fragments of the disclosure described herein comprise one or more of the following amino acid alterations: V5Q, E6Q, L11V, V12I, K13Q, R18L, K19R, V37I, E42G, A49S, T63S, A75S, F80Y, T84N, S88A, M93V, T111L or L112V, as compared with and numbered with reference to the amino acid sequence of SEQ ID NO: 17. In other words, in certain embodiments, an antibody or antigen-binding fragment comprises one or more amino acid alteration at a position corresponding to the foregoing, where the corresponding position is compared with SEQ ID NO: 17. In certain embodiments, the V_{H} domain comprises one or more of the following amino acid alterations: V5Q, L11V, K13Q, R18L, K19R, V37I, E42G, A49S, T63S, A75S, F80Y, T84N, M93V, T111L or L112V, as compared with and numbered with reference to the amino acid sequence of SEQ ID NO: 17. In certain embodiments, the V_{H} domain comprises at least 5, at least 6, at least 7, at least 8, at least 9, at least 10, at least 11, at least 12, at least 13, at least 14, at least 15, at least 16, or at least 17 of said alterations, as compared with and numbered with reference to the amino acid sequence of SEQ ID NO: 17. In certain embodiments, at least one of the alterations in the V_{H} domain is a V5Q alteration, as compared with and numbered with reference to the amino acid sequence of SEQ ID NO: 17. In certain embodiments, at least one of the alterations in the V_{H} domain is a E6Q alteration, as compared with and numbered with reference to the amino acid sequence of SEQ ID NO: 17. In certain embodiments, at least one of the alterations in the V_{H} domain is a L11V alteration, as compared with and numbered with reference to the amino acid sequence of SEQ ID NO: 17. In certain embodiments, at least one of the alterations in the V_{H} domain is a V37I alteration, as compared with and numbered with reference to the amino acid sequence of SEQ ID NO: 17. In certain embodiments, the V_{H} domain retains a serine at the amino acid position corresponding to amino acid position 88 of SEQ ID NO: 17. In certain embodiments, the V_{H} domain retains a valine at the amino acid position corresponding to amino acid position 12 of SEQ ID NO: 17. In certain embodiments, the V_{H} domain retains a tryptophan at the amino acid position corresponding to amino acid position 47 of SEQ ID NO: 17. All operable combinations of the foregoing are contemplated, as are combinations with any of the aspect and embodiments provided herein for the VL. The foregoing numbering of amino acid residues is with reference to linear amino acid sequence of a given VH and the disclosure contemplates humanized antibodies and antigen binding fragments having one or more of the recited substitutions at a position corresponding to the recited position in the murine, parent VH or VL.

In certain embodiments of any of the foregoing, or of any of the aspects and embodiments disclosed herein, the V_{L} domain of any of the humanized antibodies or antigen-binding fragments described herein comprise one or more of the following amino acid alterations: V3Q, L4M, A9S, A12S, V13A, L15V, Q17D, A19V, S22T, M37L, H38A, G45E, Q46K, P47A, E59Q, A64S, H76T, N78T, H80S, P81S, V82L, E83Q, E84P, A87V, A87F, or G104A, as compared with and numbered with reference to the amino acid sequence of SEQ ID NO: 18. In certain embodiments, the V_{L} domain comprises one or more of the following amino acid alterations: V3Q, L4M, A9S, A12S, V13A, L15V, Q17D, A19V, G45E, Q46K, P47A, E59Q, A64S, H76T, N78T, H80S, P81S, V82L, E83Q, E84P, A87V, or G104A, as compared with and numbered with reference to the amino acid sequence of SEQ ID NO: 18. In certain embodiments, the V_{L} domain comprises at least 5, at least 6, at least 7, at least 8, at least 9, at least 10, at least 11, at least 12, at least 13, at least 14, at least 15, at least 16, at least 17, at least 18, at least 19, at least 20, at least 21, or at least 22 of said amino acid alterations, as compared with and numbered with reference to the amino acid sequence of SEQ ID NO: 18.

It should be understood that any of the foregoing variations at particular positions are referred to relative to the amino acid sequence set forth in SEQ ID NO: 18 or 17. An antibody or antigen binding fragment of the disclosure may comprise one or more of such amino acid alterations at the corresponding position, relative to the amino acid sequence of SEQ ID NO: 18 or 17. By way of example, in certain embodiments, the VH domain comprises an L to V alteration at a position corresponding to position 11 of SEQ ID NO: 17 (e.g., an L11V alteration). This is exemplary of how all of the foregoing alterations can also be described, and such description is expressly contemplated. By way of another example, in certain embodiments, the VL domain comprises a V to Q alteration at a position corresponding to position 3 of SEQ ID NO: 18 (e.g., a V3Q alteration).

In certain embodiments, the V_{L} domain comprises a serine at each of the amino acid positions corresponding to amino acid positions 80 and 81 of SEQ ID NO: 18. In certain embodiments, the V_{L} domain retains a lysine at the amino acid position corresponding to amino acid position 53 of SEQ ID NO: 18. In certain embodiments, the V_{L} domain does not have any one or more of the following amino acid combinations:

a) asparagine and serine at the amino acid positions corresponding to amino acid positions 80 and 81 of SEQ ID NO: 18, respectively; or

b) asparagine and glycine at the amino acid positions corresponding to amino acid positions 80 and 81 of SEQ ID NO: 18, respectively; or

c) asparagine and proline at the amino acid positions corresponding to amino acid positions 80 and 81 of SEQ ID NO: 18, respectively. All operable combinations of the foregoing are contemplated, as are combinations with any of the aspect and embodiments provided herein for the VH. The foregoing numbering of amino acid residues is with reference to linear amino acid sequence of a given VH and the disclosure contemplates humanized antibodies and antigen binding fragments having one or more of the recited substitutions at a position corresponding to the recited position in the murine, parent VH or VL.

In some embodiments, the humanized internalizing moiety (e.g., a humanized antibody or antigen-binding fragment comprising a light chain variable (V_{L}) domain comprising the amino acid sequence set forth in SEQ ID NO: 4 and a heavy chain variable (V_{H}) domain comprising the amino acid sequence set forth in SEQ ID NO: 3) is associated with at least one superior physiological or biological property as compared to a reference non-humanized internalizing moiety (e.g., the murine, parent 3E10 antibody). In other embodiments, the humanized internalizing moiety is associated with at least two superior physiological or biological properties as compared to a reference non-humanized internalizing moiety. In other embodiments, the humanized internalizing moiety is associated with at least three superior physiological or biological properties as compared to a reference non-humanized internalizing moiety (e.g., the murine, parent 3E10 antibody). In some embodiments, the reference non-humanized internalizing moiety comprises the murine parent antibody comprising a VH comprising the amino acid sequence of SEQ ID NO: 17 and a VL comprising the amino acid sequence of SEQ ID NO: 18. In some embodiments, the reference humanized internalizing moiety is an antibody comprising the amino acid sequence of SEQ ID NO: 28. In some embodiments, the reference internalizing moiety is a humanized antibody or antigen binding fragment comprising the V_{H} amino acid sequence of SEQ ID NO: 3 and the V_{L} amino acid sequence of SEQ ID NO: 4.

In certain embodiments, the antibodies or antigen-binding fragments described herein are humanized and are associated with at least one superior biological or physiological property as compared to a murine antibody, which murine antibody comprises a V_{L} domain comprising the amino acid sequence set forth in SEQ ID NO: 18 and a V_{H} domain comprising the amino acid sequence set forth in SEQ ID NO: 17, and/or as compared to an alternative antibody or antigen-binding fragment thereof, wherein said alternative antibody or antigen-binding fragment comprises a V_{L} domain comprising the CDRs of the amino acid sequence set forth in SEQ ID NO: 18 and a V_{H} domain comprising the CDRs of the amino acid sequence set forth in SEQ ID NO: 17; and wherein said alternative antibody or fragment does not comprise a V_{L} domain comprising the amino acid sequence of SEQ ID NO: 4, and/or wherein said alternative antibody or fragment does not comprise a V_{H} domain comprising the amino acid sequence of any of SEQ ID NO: 3; or, in some embodiments, wherein said alternative antibody or fragment does not comprise a V_{L} domain comprising the amino acid sequence of SEQ ID NO: 4, and/or wherein said alternative antibody or fragment does not comprise a V_{H} domain comprising the amino acid sequence of SEQ ID NO: 3.

In some embodiments, a humanized internalizing moiety of the disclosure (e.g., a humanized antibody or antigen-binding fragment thereof comprises a light chain variable (V_{L}) domain comprising the amino acid sequence set forth in SEQ ID NO: 4 and a heavy chain variable (V_{H}) domain comprising the amino acid sequence set forth in SEQ ID NO: 3) is associated with at least one superior physiological or biological property as compared to an alternative internalizing moiety or fragment thereof (e.g., a different humanized antibody based on the same parent, murine antibody and, optionally, having the same CDRs). In other embodiments, a humanized internalizing moiety of the disclosure is associated with at least two superior physiological or biological properties as compared to the alternative internalizing moiety (e.g., a different humanized antibody based on the same parent, murine antibody and, optionally, having the same CDRs). In other embodiments, the humanized internalizing moiety of the disclosure is associated with at least three superior physiological or biological properties as compared to the alternative internalizing moiety (e.g., a different humanized antibody based on the same parent, murine antibody and, optionally, having the same CDRs). In some embodiments, the alternative antibody is the parent antibody from which the humanized antibody was derived (e.g., the parent, murine antibody). In some embodiments, the alternative antibody is another humanized antibody that is derived from the 3E10 antibody but that has a different amino acid sequence than the humanized internalizing moieties or antigen-binding fragments thereof of the present disclosure. In some embodiments, an antibody or antigen binding fragment of the disclosure has one or more improved characteristics in comparison to the murine parent antibody and/or an alternative humanized antibody. In some embodiments, the alternative humanized antibody has one, two, or three amino acid substitutions in the Kabat CDRs, as compared to an antibody of the disclosure. In some embodiments, the alternative internalizing moiety or fragment thereof comprises:

a VH CDR1 having the amino acid sequence of SEQ ID NO: 19;

a VH CDR2 having the amino acid sequence of SEQ ID NO: 20;

a VH CDR3 having the amino acid sequence of SEQ ID NO: 21;

a VL CDR1 having the amino acid sequence of SEQ ID NO: 22;

a VL CDR2 having the amino acid sequence of SEQ ID NO: 23; and

a VL CDR3 having the amino acid sequence of SEQ ID NO: 24, which CDRs are defined in accordance with Kabat, but does not comprise the same scaffold amino acid sequence present in the humanized internalizing moieties or fragments thereof of the present disclosure (e.g. a humanized internalizing moiety or fragment thereof comprising the amino acid sequence of any of SEQ ID NO: 3 or 4).

In some embodiments, the superior biological or physiological property associated with the humanized internalizing moieties or fragments of the disclosure described herein is that the humanized internalizing moiety or antigen-binding fragment is associated with reduced immunogenicity in a human patient as compared to the immunogenicity of the non-humanized or to the alternative antibody or antigen-binding fragment in a human patient. The skilled worker is familiar with numerous assays for determining the immunogenicity of the antibodies. In preferred embodiments, the humanized antibodies of the disclosure are associated with reduced immunogenicity in a human patient, but retain the cell penetration properties associated with the murine 3E10 antibody.

In some embodiments, the superior biological or physiological property associated with the humanized internalizing moieties or fragments of the disclosure described herein is that the humanized internalizing moiety or antigen-binding fragment is associated with increased solubility in a physiologically acceptable carrier as compared to the solubility of the non-humanized or to the alternative antibody or antigen-binding fragment in the same type of physiologically acceptable carrier. As used herein, a physiologically acceptable carrier includes include water, ethanol, polyols (such as glycerol, propylene glycol, polyethylene glycol, and the like), and suitable mixtures thereof, vegetable oils, such as olive oil, and injectable organic esters, such as ethyl oleate. In some embodiments, the humanized internalizing moiety or fragment is associated with at least 5%, 10%, 25%, 50%, 75%, 100%, 150%, 200% or 300% greater solubility in a physiologically acceptable carrier as compared to a non-humanized or alternative internalizing moiety or antigen-binding fragment in the same type of physiologically acceptable carrier. The skilled worker is aware of routine experiments that may be utilized for testing the solubility of the humanized internalizing moieties or fragments thereof. Examples of solubility assays include standard turbidity or light-scattering assays, commercial solubility assays, such as the OptiSol™ solubility assay kit (DiLyx, Seattle, Wash.), or the protein solubility assay screen described in Bondos et al., 2003, Analytical Biochemistry, 316:223-231 may be utilized.

In some embodiments, the superior biological or physiological property associated with the humanized internalizing moieties or fragments of the disclosure described herein is that the humanized internalizing moiety or antigen-binding fragment is associated with a higher expression level in a type of cell as compared to the expression level of the non-humanized or alternative antibody or antigen-binding fragment in the same type of cell. In some embodiments, the humanized internalizing moiety or fragment is associated with at least 5%, 10%, 25%, 50%, 75%, 100%, 150%, 200% or 300% higher expression level in a cell as compared to the expression level of a non-humanized or alternative internalizing moiety or antigen-binding fragment in the same type of cell. The skilled worker is aware of routine experiments that may be utilized for testing the expression level of the humanized internalizing moieties or fragments thereof.

In some embodiments, the superior biological or physiological property associated with the humanized internalizing moieties or fragments of the disclosure described herein is that the humanized internalizing moiety or antigen-binding fragment is associated with lower toxicity (e.g., cytotoxicity and/or genotoxicity) in a cell type as compared to the toxicity in the same type of cell that is associated with the non-humanized or alternative antibody or antigen-binding fragment. In some embodiments, the humanized internalizing moiety or fragment is associated with at least 5%, 10%, 25%, 50%, 75%, 100%, 150%, 200% or 300% lower toxicity as compared to the toxicity of a non-humanized or alternative internalizing moiety or antigen-binding fragment in the same type of cell. In some embodiments the cell is a mammalian cell. In some embodiments the cell is a human cell. In some embodiments, the cell is in an organism, such as a mammal. In some embodiments, the cell is a human cell in a human organism. The skilled worker is aware of routine experiments that may be utilized for testing the toxicity of the humanized internalizing moieties or fragments thereof. For example, the toxicity of the humanized internalizing moieties or fragments of the disclosure and of the non-humanized or alternative internalizing moieties or fragments thereof may be tested in an in vitro cell or cell culture, such as in a cell or cell culture derived from human cells, or may be tested in an in vitro animal model such as a mouse or rat.

In some embodiments, the superior biological or physiological property associated with the humanized internalizing moieties or fragments of the disclosure described herein is that the humanized internalizing moiety or antigen-binding fragment is associated with reduced aggregation in a physiologically acceptable carrier as compared to aggregation of the non-humanized or alternative antibody or antigen-binding fragment in the same type of physiologically acceptable carrier. In some embodiments, the humanized internalizing moiety or fragment is associated with at least 5%, 10%, 25%, 50%, 75%, 100%, 150%, 200% or 300% less aggregation in a physiologically acceptable carrier as compared to a non-humanized or alternative internalizing moiety or antigen-binding fragment in the same type of physiologically acceptable carrier. In some embodiments, the humanized antibody or antigen-binding fragment in a pharmaceutically acceptable carrier is associated with reduced aggregation after a period of at least 1 hour, 6 hours, 12 hours, 18 hours, 24 hours, 36 hours, 2 days, 5 days, one week, two weeks, four weeks, one month, two months, three months, six months or one year. The skilled worker is aware of routine experiments that may be utilized for testing the aggregation of the humanized internalizing moieties or fragments thereof. Examples of aggregation assays include standard turbidity or light-scattering assays (e.g., A600 nm assay), visual inspection, SDS-PAGE, commercial aggregation assays, such as the OptiSol™ aggregation assay kit (DiLyx, Seattle, Wash.), HP-SEC analysis, or the protein aggregation assay screen described in Bondos et al., 2003, Analytical Biochemistry, 316:223-231 may be utilized.

In some embodiments, the superior biological or physiological property associated with the humanized internalizing moieties or antigen-binding fragments of the disclosure described herein is that the humanized internalizing moiety or antigen-binding fragment is associated with increased stability in a physiologically acceptable carrier as compared to the stability of the non-humanized or alternative antibody or antigen-binding fragment in the same type of physiologically acceptable carrier. In some embodiments, the humanized internalizing moiety or fragment is associated with at least 5%, 10%, 25%, 50%, 75%, 100%, 150%, 200% or 300% greater stability in a physiologically acceptable carrier as compared to a non-humanized or alternative internalizing moiety or antigen-binding fragment in the same type of physiologically acceptable carrier. In some embodiments, the humanized antibody or antigen-binding fragment in a pharmaceutically acceptable carrier is associated with increased stability after a period of at least 1 hour, 6 hours, 12 hours, 18 hours, 24 hours, 36 hours, 2 days, 5 days, one week, two weeks, four weeks, one month, two months, three months, six months or one year as compared to a non-humanized or alternative internalizing moiety or antigen-binding fragment in the same type of physiologically acceptable carrier. The skilled worker is aware of routine experiments that may be utilized for testing the stability of the humanized internalizing moieties or fragments thereof. For example, the skilled worker could test the stability of the humanized and non-humanized or alternative internalizing moieties or fragments thereof after various intervals of being stored in a physiologically acceptable carrier. Commercial assays such as the ProteoStat™ Thermal shift stability assay (Enzo, Farmingdale, N.Y.) may be utilized in assessing the stability of the moieties or fragments thereof. Alternatively, the stability of the moieties or fragments thereof may be determined by HP-SEC or by SDS-PAGE analysis.

In some embodiments, the superior biological or physiological property associated with the humanized internalizing moieties or antigen-binding fragments of the disclosure described herein is that the humanized internalizing moiety or antigen-binding fragment is associated with improved cell penetration as compared to the cell penetration of the non-humanized or alternative antibody or antigen-binding fragment. In some embodiments, the improved penetration is due to the increased efficiency of the humanized internalizing moiety or antigen-binding fragment to be internalized by an ENT transporter (e.g., an ENT2 and/or ENT3 transporter). In some embodiments, the humanized internalizing moiety or fragment is associated with at least 5%, 10%, 25%, 50%, 75%, 100%, 150%, 200% or 300% greater cell penetration as compared to a non-humanized or alternative internalizing moiety or antigen-binding fragment in the same type of physiologically acceptable carrier. The skilled worker is aware of routine experiments that may be utilized for testing the cell penetration of the humanized internalizing moieties or fragments thereof. For example, the humanized internalizing moieties or fragments thereof may be labeled (e.g. fluorescently or radiolabeled) and administered to a cell or cell culture in order to determine the cell penetration of the humanized internalizing moieties or fragments thereof. Alternatively, the humanized internalizing moieties or fragments may be administered to a cell or cell culture and then detected with a secondary agent, e.g., a fluorescently labeled or radiolabeled secondary antibody, in order to determine the cell penetration of the humanized internalizing moieties or fragments thereof.

In some embodiments, the superior biological or physiological property associated with the humanized internalizing moieties or fragments of the disclosure described herein is that the humanized internalizing moiety or antigen-binding fragment is associated with reduced glycosylation in a cell type as compared to the glycosylation of the non-humanized or alternative antibody or antigen-binding fragment in the same cell type. In some embodiments, an asparagine is mutated to another amino acid residue in the VH or VL domains in order to reduce N-linked glycosylation of the humanized antibody or antibody fragment. In other embodiments, the superior biological or physiological property associated with the humanized internalizing moieties or fragments described herein is that the humanized internalizing moiety or antigen-binding fragment is associated with increased glycosylation in a cell type as compared to the glycosylation of the non-humanized or alternative antibody or antigen-binding fragment in the same cell type. In some embodiments, the superior biological or physiological property associated with the humanized internalizing moieties or fragments described herein is that the humanized internalizing moiety or antigen-binding fragment is associated with a specific pattern of glycosylation in a cell type that differs from the glycosylation pattern of the non-humanized or alternative internalizing moiety or antigen-binding fragment in the same type of cell. For example, the humanized internalizing moiety or antigen-binding fragment may be hemi-glycosylated in a cell type while the non-humanized or alternative internalizing moiety or antigen-binding fragment is not hemi-glycosylated in the same type of cell. In some embodiments, the superior biological or physiological property associated with the humanized internalizing moieties or fragments described herein is that the humanized internalizing moiety or antigen-binding fragment is post-translationally modified with a specific glycosylation group in a cell type that differs from the post-translational modification of the non-humanized or alternative internalizing moiety or antigen-binding fragment in the same type of cell. The skilled worker is aware of routine experiments that may be utilized for testing the glycosylation patterns of the humanized internalizing moieties or fragments thereof. Examples of experiments for testing the glycosylation levels and patterns of the internalizing moieties and fragments thereof include protocols described in Mohammad, 2002, Protein Protocols Handbook, pages 795-802; standard procedures involving mass spectrometry and/or HPLC; GLYCO-PRO™ (Sigma-Aldrich); and Qproteome Total Glycoprotein Kit™ (Qiagen, Valencia, Calif.). In order to identify the exact sites of glycosylation in a protein sequence, standard endoproteinase cleavage may be performed (e.g. tryptic digest) followed by analysis by LC/MS or HILIC-MS/MS, similar to the protocols described in Zauner G et al., 2010, J Sep Sci., 33:903-10.

In some embodiments, the superior biological or physiological property associated with the humanized internalizing moieties or fragments of the disclosure described herein is that the humanized internalizing moiety or antigen-binding fragment is associated with reduced deamidation in a physiologically acceptable carrier as compared to deamidation of the non-humanized or alternative antibody or antigen-binding fragment in the same type of physiologically acceptable carrier. In some embodiments, the humanized internalizing moiety or fragment is associated with at least 5%, 10%, 25%, 50%, 75%, 100%, 150%, 200% or 300% less deamidation in a physiologically acceptable carrier as compared to a non-humanized or alternative internalizing moiety or antigen-binding fragment in the same type of physiologically acceptable carrier. In some embodiments, the humanized antibody or antigen-binding fragment in a pharmaceutically acceptable carrier is associated with reduced deamidation after a period of at least 1 hour, 6 hours, 12 hours, 18 hours, 24 hours, 36 hours, 2 days, 5 days, one week, two weeks, four weeks, one month, two months, three months, six months or one year as compared to a non-humanized or alternative internalizing moiety or antigen-binding fragment in the same type of physiologically acceptable carrier. The skilled worker is aware of routine experiments that may be utilized for testing the deamidation of the humanized internalizing moieties or fragments thereof. Examples of assays for testing protein deamidation include commercially available deamidation assays such as the ISOQUANT® Isoaspartate Detection Kit (Promega, Madison Wis.) or Dionex UltiMate 3000 Titanium System (Dionex, Sunnyvale, Calif.). Other assays may include peptide mapping. See generally, Kalgahtgi, K., & Horvath, C. “Rapid Peptide Mapping by High Performance Liquid Chromatography”, J. Chromatography 443, 343-354 (1988).

In some embodiments, the superior biological or physiological property associated with the humanized internalizing moieties or fragments of the disclosure described herein is that the humanized internalizing moiety or antigen-binding fragment is associated with reduced oxidation in a physiologically acceptable carrier as compared to oxidation of the non-humanized or alternative antibody or antigen-binding fragment in the same type of physiologically acceptable carrier. In some embodiments, the humanized internalizing moiety or fragment is associated with at least 5%, 10%, 25%, 50%, 75%, 100%, 150%, 200% or 300% less oxidation in a physiologically acceptable carrier as compared to a non-humanized or alternative internalizing moiety or antigen-binding fragment in the same type of physiologically acceptable carrier. In some embodiments, the humanized antibody or antigen-binding fragment in a pharmaceutically acceptable carrier is associated with reduced oxidation after a period of at least 1 hour, 6 hours, 12 hours, 18 hours, 24 hours, 36 hours, 2 days, 5 days, one week, two weeks, four weeks, one month, two months, three months, six months or one year as compared to a non-humanized or alternative internalizing moiety or antigen-binding fragment in the same type of physiologically acceptable carrier. The skilled worker is aware of routine experiments that may be utilized for testing the oxidation of the humanized internalizing moieties or fragments thereof. For example, oxidation levels may be assessed by using any one of several commercially available oxidation assays, such as the Methionine Sulfoxide Immunoblotting Kit (Cayman Chemical, Ann Arbor, Mich.). Other assays may include peptide mapping. See generally, Kalgahtgi, K., & Horvath, C. “Rapid Peptide Mapping by High Performance Liquid Chromatography”, J. Chromatography 443, 343-354 (1988).

In some embodiments, the superior biological or physiological property associated with the humanized internalizing moieties or fragments of the disclosure described herein is that the humanized internalizing moiety or antigen-binding fragment is associated with reduced lipidation when produced in a cell type as compared to the lipidation of the non-humanized or alternative antibody or fragment when produced in the same type of cell. In other embodiments, the superior biological or physiological property associated with the humanized internalizing moieties or fragments described herein is that the humanized internalizing moiety or antigen-binding fragment is associated with increased lipidation when produced in a cell type as compared to the lipidation of the non-humanized or alternative antibody or antigen-binding fragment when produced in the same type of cell. In some embodiments, the superior biological or physiological property associated with the humanized internalizing moieties or fragments described herein is that the humanized internalizing moiety or antigen-binding fragment is associated with a specific pattern of lipidation when produced in a cell type that differs from the lipidation pattern of the non-humanized or alternative internalizing moiety or antigen-binding fragment when produced in the same type of cell. In some embodiments, the superior biological or physiological property associated with the humanized internalizing moieties or fragments described herein is that the humanized internalizing moiety or antigen-binding fragment is post-translationally modified with a specific lipidation group when produced in a cell type that differs from the post-translational modification of the non-humanized or alternative internalizing moiety or antigen-binding fragment when produced in the same type of cell. The skilled worker is aware of routine experiments that may be utilized for testing the lipidation patterns of the humanized internalizing moieties or fragments thereof. For example, the internalizing moieties or fragments thereof may be assessed by the protocols described in Gelb et al., 1999, Protein Lipidation Protocols, Humana Press, pages 1-256.

In some embodiments, the superior biological or physiological property associated with the humanized internalizing moieties or fragments of the disclosure described herein is that the humanized internalizing moiety or antigen-binding fragment is capable of binding a polynucleotide (e.g., DNA) with higher affinity (lower K_{D}) as compared to the binding affinity of the non-humanized, parent antibody or an alternative antibody or fragment, such as a different humanized antibody. In some embodiments, the humanized internalizing moiety or fragment is associated with at least 5%, 10%, 25%, 50%, 75%, 100%, 150%, 200% or 300% stronger binding affinity for a polynucleotide (e.g., DNA; double stranded blunt DNA) as compared to a non-humanized or alternative internalizing moiety or antigen-binding fragment in the same type of physiologically acceptable carrier. The skilled worker is aware of routine experiments that may be utilized for testing the binding affinity (K_{D}) of the humanized internalizing moieties or fragments thereof. Binding affinity can be measured using Surface Plasmon Resonance (SPR) or Quartz Crystal Microbalance (QCM), in accordance with currently standard methods and the manufacturer's protocols.

### III. Chimeric Polypeptides

The disclosure provides chimeric polypeptides comprising an internalizing moiety portion and a non-internalizing moiety portion. As detailed above, the non-internalizing moiety polypeptide portion comprises or consists of a neprilysin polypeptide. Numerous examples of internalizing moieties, and each of the potential non-internalizing moiety polypeptide portions are described above, and all suitable combinations of internalizing moiety portions and non-internalizing moiety polypeptide portions to generate chimeric polypeptides are contemplated.

Without being bound by theory, the association of the neprilysin polypeptide with the internalizing moiety portion facilitates delivery of the chimeric polypeptide, and thus, the non-internalizing moiety portion to the cytoplasm and, optionally, to the lysosome and/or autophagic vesicles. In certain embodiments, the internalizing moiety delivers neprilysin activity into cells. In certain embodiments, the chimeric polypeptide of the disclosure comprises an neprilysin-containing chimeric polypeptide (e.g., the non-internalizing moiety portion comprises or consists of a neprilysin polypeptide). Any of the internalizing moieties described herein may be combined with any of the non-internalizing moiety polypeptide portions, as described herein, to generate a chimeric polypeptide of the disclosure.

The disclosure provides chimeric polypeptides (e.g., chimeric polypeptides of the disclosure). Chimeric polypeptides for use in the methods disclosed herein can be made in various manners. The chimeric polypeptides may comprise any of the internalizing moiety portions and the neprilysin polypeptide portions disclosed herein. As used herein, chimeric polypeptides of the disclosure comprise (i) a neprilysin polypeptide portion and (ii) an internalizing moiety portion. In addition, any of the chimeric polypeptides disclosed herein may be utilized in any of the methods or compositions disclosed herein. In some embodiments, an internalizing moiety (e.g. an antibody or antigen-binding fragment) is linked, directly or indirectly, to any of the neprilysin polypeptides and/or fragments and/or variants disclosed herein.

In some embodiments, the neprilysin polypeptide is a neprilysin ectodomain polypeptide and comprises the amino acid sequence of SEQ ID NO: 2, or variants or fragments thereof, fused to the C-terminus of an internalizing moiety. In some embodiments, the neprilysin polypeptide comprises the amino acid sequence of SEQ ID NO: 2, or variants or fragments thereof, fused to the C-terminus of the heavy chain segment of a Fab internalizing moiety. In some embodiments, the neprilysin polypeptide comprises the amino acid sequence of SEQ ID NO: 2, or variants or fragments thereof, fused to the C-terminus of the heavy chain segment of a full-length antibody internalizing moiety.

In some embodiments, the neprilysin polypeptide is a neprilysin variant ectodomain polypeptide and comprises the amino acid sequence of SEQ ID NO: 31, or variants or fragments thereof, fused to the C-terminus of an internalizing moiety. In some embodiments, the neprilysin polypeptide comprises the amino acid sequence of SEQ ID NO: 31, or variants or fragments thereof, fused to the C-terminus of the heavy chain segment of a Fab internalizing moiety. In some embodiments, the neprilysin polypeptide comprises the amino acid sequence of SEQ ID NO: 31, or variants or fragments thereof, fused to the C-terminus of the heavy chain segment of a full-length antibody internalizing moiety.

In some embodiments, the chimeric polypeptide comprises: (i) a neprilysin polypeptide, and (ii) an internalizing moiety; wherein the neprilysin polypeptide comprises an amino acid sequence that is at least 85%, 90%, 91%, 92%, 93%, 94%, 95%, 96%, 97%, 98%, 99% or 100% identical to the amino acid sequence of SEQ ID NO: 2; and wherein the internalizing moiety is an antibody or antigen binding fragment, wherein the antibody or antigen binding fragment comprises a heavy chain variable domain and a light chain variable domain; wherein the heavy chain variable domain comprises an amino acid sequence that is at least 85%, 90%, 91%, 92%, 93%, 94%, 95%, 96%, 97%, 98%, 99% or 100% identical to the amino acid sequence of SEQ ID NO: 3; and wherein the light chain variable domain comprises an amino acid sequence that is at least 85%, 90%, 91%, 92%, 93%, 94%, 95%, 96%, 97%, 98%, 99% or 100% identical to the amino acid sequence of SEQ ID NO: 4. In some embodiments, the chimeric polypeptide comprises: (i) a neprilysin polypeptide, and (ii) an internalizing moiety; wherein the neprilysin polypeptide comprises the amino acid sequence of SEQ ID NO: 2; and wherein the internalizing moiety is an antibody or antigen binding fragment, wherein the antibody or antigen binding fragment comprises a heavy chain variable domain and a light chain variable domain; wherein the heavy chain variable domain comprises the amino acid sequence of SEQ ID NO: 3; and wherein the light chain variable domain comprises the amino acid sequence of SEQ ID NO: 4. In some embodiments, the heavy chain comprises a leader sequence. In some embodiments, the light chain comprises a leader sequence. In some embodiments, the disclosure provides a chimeric polypeptide that does not include a leader sequence. In some embodiments, the chimeric polypeptide comprises a linker interconnecting the neprilysin polypeptide to the internalizing moiety. In some embodiments, the linker comprises the amino acid sequence of SEQ ID NO: 5. In some embodiments, the chimeric polypeptide comprises a heavy chain amino acid sequence lacking a leader sequence. In some embodiments, the chimeric polypeptide comprises the amino acid sequence of SEQ ID NO: 6. In some embodiments, the chimeric polypeptide comprises a light chain amino acid sequence lacking a leader sequence. In some embodiments, the chimeric polypeptide comprises the amino acid sequence of SEQ ID NO: 7. In some embodiments, the chimeric polypeptide comprises the amino acid sequence of both SEQ ID NOs: 6 and 7.

In some embodiments, the chimeric polypeptide comprises an amino acid sequence that is at least 85%, 90%, 91%, 92%, 93%, 94%, 95%, 96%, 97%, 98%, 99% or 100% identical to the amino acid sequence of SEQ ID NO: 29. In some embodiments, the chimeric polypeptide comprises an amino acid sequence that is at least 85%, 90%, 91%, 92%, 93%, 94%, 95%, 96%, 97%, 98%, 99% or 100% identical to the amino acid sequence of SEQ ID NO: 7. In some embodiments, the chimeric polypeptide comprises the amino acid sequences of both SEQ ID NO: 29 and 7.

In certain embodiments, potential configurations include the use of truncated portions of an antibody's heavy and light chain sequences (e.g., mAB 3E10) as needed to maintain the functional integrity of the attached neprilysin. Further still, the internalizing moiety can be linked to an exposed internal (non-terminus) residue of neprilysin or a fragment and/or variant thereof. In some embodiments, any combination of the neprilysin-internalizing moiety configurations can be employed, thereby resulting in a neprilysin:internalizing moiety ratio that is greater than 1:1 (e.g., two neprilysin molecules to one internalizing moiety).

The neprilysin polypeptide and the internalizing moiety may be linked directly to each other. Alternatively, they may be linked to each other via a linker sequence, which separates neprilysin polypeptide and the internalizing moiety by a distance sufficient to ensure that each domain properly folds into its secondary and tertiary structures. Preferred linker sequences (1) should adopt a flexible extended conformation, (2) should not exhibit a propensity for developing an ordered secondary structure which could interact with the functional domains of the neprilysin polypeptide or the internalizing moiety, and (3) should have minimal hydrophobic or charged character, which could promote interaction with the functional protein domains. Typical surface amino acids in flexible protein regions include Gly, Asn and Ser. Permutations of amino acid sequences containing Gly, Asn and Ser would be expected to satisfy the above criteria for a linker sequence. Other near neutral amino acids, such as Thr and Ala, can also be used in the linker sequence. In a specific embodiment, a linker sequence length of about 20 amino acids can be used to provide a suitable separation of functional protein domains, although longer or shorter linker sequences may also be used. The length of the linker sequence separating the neprilysin polypeptide from the internalizing moiety can be from 5 to 500 amino acids in length, or more preferably from 5 to 100 amino acids in length. Preferably, the linker sequence is from about 5-30 amino acids in length. In preferred embodiments, the linker sequence is from about 5 to about 20 amino acids, and is advantageously from about 10 to about 20 amino acids. In other embodiments, the linker joining the neprilysin polypeptide to an internalizing moiety can be a constant domain of an antibody (e.g., constant domain of mAb 3E10 or all or a portion of an Fc region of another antibody). In certain embodiments, the linker is a cleavable linker. In certain embodiments, the linker sequence comprises the linker sequence of SEQ ID NO: 5. In certain embodiments, the internalizing moiety is an antibody or antibody fragment and the conjugation includes chemical or recombinant conjugation to a constant domain, such as the constant domain of a heavy chain of the antibody or antibody fragment. In such embodiments, it is appreciated that the neprilysin polypeptide and internalizing moiety may be further associated via the association between the heavy chain and light chain of the antibody or antibody fragment. This is also included within the scope of the conjugation.

In other embodiments, the neprilysin polypeptide or functional fragment thereof may be conjugated or joined directly to the internalizing moiety. For example, a recombinantly conjugated chimeric polypeptide can be produced as an in-frame fusion of the neprilysin portion and the internalizing moiety portion. In certain embodiments, the linker may be a cleavable linker. In any of the foregoing embodiments, the internalizing moiety may be conjugated (directly or via a linker) to the N-terminal or C-terminal amino acid of the neprilysin polypeptide. In other embodiments, the internalizing moiety may be conjugated (directly or indirectly) to an internal amino acid of the neprilysin polypeptide. Note that the two portions of the construct are conjugated/joined to each other. Unless otherwise specified, describing the chimeric polypeptide as a conjugation of the neprilysin portion to the internalizing moiety is used equivalently as a conjugation of the internalizing moiety to the neprilysin portion. Further, unless otherwise specified, conjugation and/or joining refers to either chemical or genetic conjugation.

In certain embodiments, the chimeric polypeptides of the present disclosure can be generated using well-known cross-linking reagents and protocols. For example, there are a large number of chemical cross-linking agents that are known to those skilled in the art and useful for cross-linking the neprilysin polypeptide with an internalizing moiety (e.g., an antibody). For example, the cross-linking agents are heterobifunctional cross-linkers, which can be used to link molecules in a stepwise manner. Heterobifunctional cross-linkers provide the ability to design more specific coupling methods for conjugating proteins, thereby reducing the occurrences of unwanted side reactions such as homo-protein polymers. A wide variety of heterobifunctional cross-linkers are known in the art, including succinimidyl 4-(N-maleimidomethyl) cyclohexane-1-carboxylate (SMCC), m-Maleimidobenzoyl-N-hydroxysuccinimide ester (MBS); N-succinimidyl (4-iodoacetyl) aminobenzoate (SIAB), succinimidyl 4-(p-maleimidophenyl) butyrate (SMPB), 1-ethyl-3-(3-dimethylaminopropyl) carbodiimide hydrochloride (EDC); 4-succinimidyloxycarbonyl-a-methyl-a-(2-pyridyldithio)-tolune (SMPT), N-succinimidyl 3-(2-pyridyldithio) propionate (SPDP), succinimidyl 6-[3-(2-pyridyldithio) propionate] hexanoate (LC-SPDP). Those cross-linking agents having N-hydroxysuccinimide moieties can be obtained as the N-hydroxysulfosuccinimide analogs, which generally have greater water solubility. In addition, those cross-linking agents having disulfide bridges within the linking chain can be synthesized instead as the alkyl derivatives so as to reduce the amount of linker cleavage in vivo. In addition to the heterobifunctional cross-linkers, there exist a number of other cross-linking agents including homobifunctional and photoreactive cross-linkers. Disuccinimidyl subcrate (DSS), bismaleimidohexane (BMH) and dimethylpimelimidate.2 HCl (DMP) are examples of useful homobifunctional cross-linking agents, and bis-[B-(4-azidosalicylamido)ethyl]disulfide (BASED) and N-succinimidyl-6(4′-azido-2′-nitrophenylamino)hexanoate (SANPAH) are examples of useful photoreactive cross-linkers for use in this disclosure. For a recent review of protein coupling techniques, see Means et al. (1990) Bioconjugate Chemistry. 1:2-12, incorporated by reference herein.

One particularly useful class of heterobifunctional cross-linkers, included above, contain the primary amine reactive group, N-hydroxysuccinimide (NHS), or its water soluble analog N-hydroxysulfosuccinimide (sulfo-NHS). Primary amines (lysine epsilon groups) at alkaline pH's are unprotonated and react by nucleophilic attack on NHS or sulfo-NHS esters. This reaction results in the formation of an amide bond, and release of NHS or sulfo-NHS as a by-product. Another reactive group useful as part of a heterobifunctional cross-linker is a thiol reactive group. Common thiol reactive groups include maleimides, halogens, and pyridyl disulfides. Maleimides react specifically with free sulfhydryls (cysteine residues) in minutes, under slightly acidic to neutral (pH 6.5-7.5) conditions. Halogens (iodoacetyl functions) react with —SH groups at physiological pH's. Both of these reactive groups result in the formation of stable thioether bonds. The third component of the heterobifunctional cross-linker is the spacer arm or bridge. The bridge is the structure that connects the two reactive ends. The most apparent attribute of the bridge is its effect on steric hindrance. In some instances, a longer bridge can more easily span the distance necessary to link two complex biomolecules.

In some embodiments, the chimeric polypeptide comprises multiple linkers. For example, if the chimeric polypeptide comprises an scFv internalizing moiety, the chimeric polypeptide may comprise a first linker conjugating the neprilysin to the internalizing moiety, and a second linker in the scFv conjugating the V_{H} domain (e.g., SEQ ID NO: 3) to the V_{L} domain (e.g., SEQ ID NO: 4).

Preparing protein-conjugates using heterobifunctional reagents is a two-step process involving the amine reaction and the sulfhydryl reaction. For the first step, the amine reaction, the protein chosen should contain a primary amine. This can be lysine epsilon amines or a primary alpha amine found at the N-terminus of most proteins. The protein should not contain free sulfhydryl groups. In cases where both proteins to be conjugated contain free sulfhydryl groups, one protein can be modified so that all sulfhydryls are blocked using for instance, N-ethylmaleimide (see Partis et al. (1983) J. Pro. Chem. 2:263, incorporated by reference herein). Ellman's Reagent can be used to calculate the quantity of sulfhydryls in a particular protein (see for example Ellman et al. (1958) Arch. Biochem. Biophys. 74:443 and Riddles et al. (1979) Anal. Biochem. 94:75, incorporated by reference herein).

In certain specific embodiments, chimeric polypeptides of the disclosure can be produced by using a universal carrier system. For example, a neprilysin polypeptide can be conjugated to a common carrier such as protein A, poly-L-lysine, hex-histidine, and the like. The conjugated carrier will then form a complex with an antibody which acts as an internalizing moiety. A small portion of the carrier molecule that is responsible for binding immunoglobulin could be used as the carrier.

In certain embodiments, chimeric polypeptides of the disclosure can be produced by using standard protein chemistry techniques such as those described in Bodansky, M. Principles of Peptide Synthesis, Springer Verlag, Berlin (1993) and Grant G. A. (ed.), Synthetic Peptides: A User's Guide, W. H. Freeman and Company, New York (1992). In addition, automated peptide synthesizers are commercially available (e.g., Advanced ChemTech Model 396; Milligen/Biosearch 9600). In any of the foregoing methods of cross-linking for chemical conjugation of neprilysin to an internalizing moiety, a cleavable domain or cleavable linker can be used. Cleavage will allow separation of the internalizing moiety and the neprilysin polypeptide. For example, following penetration of a cell by a chimeric polypeptide, cleavage of the cleavable linker would allow separation of neprilysin from the internalizing moiety.

In certain embodiments, the chimeric polypeptides comprising a neprilysin polypeptide and an internalizing moiety portion can be generated as a fusion protein containing the neprilysin polypeptide and the internalizing moiety. In certain embodiments, the chimeric polypeptides of the present disclosure can be generated as a fusion protein containing a neprilysin polypeptide and an internalizing moiety (e.g., an antibody or a homing peptide), expressed as one contiguous polypeptide chain. In certain embodiments, the chimeric polypeptide is generated as a fusion protein that comprises a neprilysin polypeptide portion and internalizing moiety portion. In preparing such fusion protein, a fusion gene is constructed comprising nucleic acids which encode a neprilysin polypeptide and an internalizing moiety, and optionally, a peptide linker sequence to span the neprilysin polypeptide and the internalizing moiety. The use of recombinant DNA techniques to create a fusion gene, with the translational product being the desired fusion protein, is well known in the art. Both the coding sequence of a gene and its regulatory regions can be redesigned to change the functional properties of the protein product, the amount of protein made, or the cell type in which the protein is produced. The coding sequence of a gene can be extensively altered—for example, by fusing part of it to the coding sequence of a different gene to produce a novel hybrid gene that encodes a fusion protein. Examples of methods for producing fusion proteins are described in PCT applications PCT/US87/02968, PCT/US89/03587 and PCT/US90/07335, as well as Traunecker et al. (1989) Nature 339:68, incorporated by reference herein. Essentially, the joining of various DNA fragments coding for different polypeptide sequences is performed in accordance with conventional techniques, employing blunt-ended or stagger-ended termini for ligation, restriction enzyme digestion to provide for appropriate termini, filling in of cohesive ends as appropriate, alkaline phosphatase treatment to avoid undesirable joining, and enzymatic ligation. Alternatively, the fusion gene can be synthesized by conventional techniques including automated DNA synthesizers. In another method, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, Eds. Ausubel et al. John Wiley & Sons: 1992). The chimeric polypeptides encoded by the fusion gene may be recombinantly produced using various expression systems as is well known in the art (also see below).

Recombinantly conjugated chimeric polypeptides include embodiments in which the neprilysin polypeptide is conjugated to the N-terminus or C-terminus of the internalizing moiety. Exemplary chimeric polypeptides in which neprilysin is conjugated to variant light and heavy chains of Fv3E10 are indicated in SEQ ID NO: 4 and 3, respectively.

Recombinantly conjugated chimeric polypeptides include embodiments in which the internalizing moiety is N-terminal to the neprilysin polypeptide and embodiments in which the internalizing moiety is C-terminal to the neprilysin polypeptide portion. We note that methods of making fusion proteins recombinantly are well known in the art. Any of the chimeric proteins described herein can readily be made recombinantly. This includes proteins having one or more tags and/or one or more linkers. For example, if the chimeric polypeptide comprises an scFv internalizing moiety, the chimeric polypeptide may comprise a first linker interconnection the internalizing moiety to the neprilysin polypeptide portion, and a second linker in the scFv conjugating the V_{H} domain.

In some embodiments, the chimeric polypeptides are produced recombinantly in cells. In some embodiments, the cells are bacteria (e.g., E. coli), yeast (e.g., Picchia), insect cells (e.g., Sf9 cells) or mammalian cells (e.g., CHO or HEK-293 cells). Chimeric polypeptides of the disclosure are, in certain embodiments, made in any of the foregoing cells in culture using art recognized techniques for making and purifying protein from cells or cell supernatant.

The presence in the chimeric polypeptide of all or a portion of an immunoglobulin or an epitope tag, such as an HA or myc tag, provides a region for purification of chimeric polypeptide.

In some embodiments, the immunogenicity of the chimeric polypeptide may be reduced by identifying a candidate T-cell epitope within a junction region spanning the chimeric polypeptide and changing an amino acid within the junction region as described in U.S. Patent Publication No. 2003/0166877.

Chimeric polypeptides according to the disclosure can be used for numerous purposes. We note that any of the chimeric polypeptides described herein can be used in any of the methods described herein, and such suitable combinations are specifically contemplated.

Chimeric polypeptides described herein can be used to deliver neprilysin polypeptide to cells, particularly to a muscle cell. In certain embodiments, chimeric polypeptides deliver neprilysin to retinal cells. Thus, the chimeric polypeptides can be used to facilitate transport of neprilysin to cells in vitro or in vivo. By facilitating transport to cells, the chimeric polypeptides improve delivery efficiency, thus facilitating working with neprilysin polypeptide in vitro or in vivo. Further, by increasing the efficiency of transport, the chimeric polypeptides may help decrease the amount of neprilysin needed for in vitro or in vivo experimentation. Moreover, by facilitating cell penetration, the chimeric polypeptides and methods of the disclosure can address the problems associated with offending protein aggregation and inclusion body formation, for example, from protein aggregation-based disorders.

The chimeric polypeptides can be used to study the function of neprilysin in cells in culture, as well as to study transport of neprilysin. The chimeric polypeptides can be used to identify binding partners for neprilysin in cells, such as transport between cytoplasm and lysosome. The chimeric polypeptides can be used in screens to identify modifiers (e.g., small organic molecules or polypeptide modifiers) of neprilysin activity in a cell. The chimeric polypeptides can be used to help treat or alleviate the symptoms of protein aggregation-based disorders in humans or in an animal model. The foregoing are merely exemplary uses for the subject chimeric polypeptides.

Any of the chimeric polypeptides described herein, including chimeric polypeptides combining any of the features of the neprilysin polypeptides, internalizing moieties, and linkers, may be used in any of the methods of the disclosure.

### IV. Neprilysin-Related Nucleic Acids and Expression

In certain embodiments, the present disclosure makes use of nucleic acids for producing a neprilysin polypeptide (including a neprilysin ectodomain polypeptide and functional fragments, variants, and fusions thereof). In certain specific embodiments, the nucleic acids may further comprise DNA which encodes an internalizing moiety for making a recombinant chimeric protein of the disclosure.

In certain embodiments, the disclosure relates to isolated or recombinant nucleic acid sequences that are at least 80%, 85%, 90%, 95%, 97%, 98%, 99% or 100% identical to a region of a neprilysin nucleotide sequence (e.g., residues 52-750 of GenBank Accession No. NP_001341571.1). In some embodiments, the nucleotide sequence encodes a neprilysin ectodomain polypeptide sequence. In particular embodiments, the neprilysin nucleotide sequence encodes a neprilysin polypeptide that lacks the amino acids corresponding to amino acids 1-51 of SEQ ID NO: 1. In further embodiments, the neprilysin nucleic acid sequences can be isolated, recombinant, and/or fused with a heterologous nucleotide sequence, or in a DNA library.

In certain embodiments, neprilysin nucleic acids also include nucleotide sequences that hybridize under highly stringent conditions to any of the above-mentioned nucleotide sequences, or complement sequences thereof. One of ordinary skill in the art will understand readily that appropriate stringency conditions which promote DNA hybridization can be varied. For example, one could perform the hybridization at 6.0×sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the disclosure provides nucleic acids which hybridize under low stringency conditions of 6×SSC at room temperature followed by a wash at 2×SSC at room temperature.

Isolated nucleic acids which differ from the native neprilysin nucleic acids due to degeneracy in the genetic code are also within the scope of the disclosure. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the nucleotides) of the nucleic acids encoding a particular protein may exist among individuals of a given species due to natural allelic variation. Any and all such nucleotide variations and resulting amino acid polymorphisms are within the scope of this disclosure.

In some embodiments, any of the nucleic acids disclosed herein are codon optimized for expression in a particular cell expression system, e.g., a mammalian cell, a yeast cell, a bacterial cell, a plant cell or an insect cell. In some embodiments, the nucleic acids are codon optimized for expression in a mammalian cell, such as a CHO or HEK-293 cell.

In certain embodiments, the recombinant neprilysin nucleic acids may be operably linked to one or more regulatory nucleotide sequences in an expression construct. Regulatory nucleotide sequences will generally be appropriate for a host cell used for expression. Numerous types of appropriate expression vectors and suitable regulatory sequences are known in the art for a variety of host cells. Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the disclosure. The promoters may be either naturally occurring promoters, or hybrid promoters that combine elements of more than one promoter. An expression construct may be present in a cell on an episome, such as a plasmid, or the expression construct may be inserted in a chromosome. In a preferred embodiment, the expression vector contains a selectable marker gene to allow the selection of transformed host cells. Selectable marker genes are well known in the art and will vary with the host cell used. In certain aspects, this disclosure relates to an expression vector comprising a nucleotide sequence encoding a neprilysin polypeptide, such as any of the neprilysin polypeptides described herein, and operably linked to at least one regulatory sequence. Regulatory sequences are art-recognized and are selected to direct expression of the encoded polypeptide. Accordingly, the term regulatory sequence includes promoters, enhancers, and other expression control elements. Exemplary regulatory sequences are described in Goeddel; Gene Expression Technology: Methods in Enzymology, Academic Press, San Diego, Calif. (1990). It should be understood that the design of the expression vector may depend on such factors as the choice of the host cell (e.g., Chinese Hamster Ovary cells) to be transformed and/or the type of protein desired to be expressed. Moreover, the vector's copy number, the ability to control that copy number and the expression of any other protein encoded by the vector, such as antibiotic markers, should also be considered.

In some embodiments, a nucleic acid construct, comprising a nucleotide sequence that encodes a neprilysin polypeptide or a bioactive fragment thereof, is operably linked to a nucleotide sequence that encodes an internalizing moiety, wherein the nucleic acid construct encodes a chimeric polypeptide having neprilysin biological activity. In certain embodiments, the nucleic acid constructs may further comprise a nucleotide sequence that encodes a linker.

This disclosure also pertains to a host cell transfected with a recombinant gene which encodes a neprilysin polypeptide or a chimeric polypeptide of the disclosure. The host cell may be any prokaryotic or eukaryotic cell. For example, a neprilysin polypeptide or a chimeric polypeptide may be expressed in bacterial cells such as E. coli, insect cells (e.g., using a baculovirus expression system), yeast, or mammalian cells. Other suitable host cells are known to those skilled in the art.

The present disclosure further pertains to methods of producing a neprilysin polypeptide or a chimeric polypeptide of the disclosure. For example, a host cell transfected with an expression vector encoding a neprilysin polypeptide or a chimeric polypeptide can be cultured under appropriate conditions to allow expression of the polypeptide to occur. The polypeptide may be secreted and isolated from a mixture of cells and medium containing the polypeptides. Alternatively, the polypeptides may be retained cytoplasmically or in a membrane fraction and the cells harvested, lysed and the protein isolated. A cell culture includes host cells, media and other byproducts. Suitable media for cell culture are well known in the art. The polypeptides can be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the polypeptides (e.g., a neprilysin polypeptide). In a preferred embodiment, the polypeptide is a fusion protein containing a domain which facilitates its purification.

A recombinant neprilysin nucleic acid can be produced by ligating the cloned gene, or a portion thereof, into a vector suitable for expression in either prokaryotic cells, eukaryotic cells (yeast, avian, insect or mammalian), or both. Expression vehicles for production of a recombinant polypeptide include plasmids and other vectors. For instance, suitable vectors include plasmids of the types: pBR322-derived plasmids, pEMBL-derived plasmids, pEX-derived plasmids, pBTac-derived plasmids and pUC-derived plasmids for expression in prokaryotic cells, such as E. coli. The preferred mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant polypeptide by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al., John Wiley & Sons: 1992).

The disclosure contemplates methods of producing chimeric proteins recombinantly, such as described above. Suitable vectors and host cells may be readily selected for expression of proteins in, for example, yeast or mammalian cells. Host cells may express a vector encoding a chimeric polypeptide stably or transiently. Such host cells may be cultured under suitable conditions to express chimeric polypeptide which can be readily isolated from the cell culture medium.

Chimeric polypeptides of the disclosure (e.g., polypeptides comprising a neprilysin ectodomain portion and an internalizing moiety portion) may be expressed as a single polypeptide chain or as more than one polypeptide chains. An example of a single polypeptide chain is when a neprilysin portion is fused inframe to an internalizing moiety, which internalizing moiety is an scFv. In certain embodiments, this single polypeptide chain is expressed from a single vector as a fusion protein.

An example of more than one polypeptide chains is when the internalizing moiety is an antibody or Fab. In certain embodiments, the heavy and light chains of the antibody or Fab may be expressed in a host cell expressing a single vector or two vectors (one expressing the heavy chain and one expressing the light chain). In either case, the neprilysin polypeptide may be expressed as an inframe fusion to, for example, the C-terminus of the heavy chain such that the neprilysin polypeptide is appended to the internalizing moiety but at a distance to the antigen binding region of the internalizing moiety.

As noted above, methods for recombinantly expressing polypeptides, including chimeric polypeptides, are well known in the art. Nucleotide sequences expressing a mature neprilysin polypeptide, such as a human neprilysin ectodomain polypeptide, having a particular amino acid sequence are available and can be used. Moreover, nucleotide sequences expressing an internalizing moiety portion, such as expressing a 3E10 antibody, scFv, or Fab comprising the VH and VL set forth in SEQ ID NO: 3 and 4 are publicly available and can be combined with nucleotide sequence encoding suitable heavy and light chain constant regions. The disclosure contemplates nucleotide sequences encoding any of the chimeric polypeptides of the disclosure, vectors (single vector or set of vectors) comprising such nucleotide sequences, host cells comprising such vectors, and methods of culturing such host cells to express chimeric polypeptides of the disclosure.

### V. Methods of Treatment and Other Methods of Use

For any of the methods described herein, the disclosure contemplates the use of any of the chimeric polypeptides and/or compositions described throughout the application. In addition, for any of the methods described herein, the disclosure contemplates the combination of any step or steps of one method with any step or steps from another method.

For example, a chimeric polypeptide of the disclosure comprising a neprilysin polypeptide (e.g., a neprilysin ectodomain polypeptide) portion and an internalizing moiety portion can be used in any of the methods of the disclosure.

In certain embodiments, a chimeric polypeptide of the disclosure (e.g., a polypeptide comprising a neprilysin polypeptide portion and an internalizing moiety portion) is delivered to the cytoplasm of cells, such as muscle (e.g., diaphragm muscle, skeletal, and/or cardiac muscle), neuronal cells (e.g., neuronal cells of the brain) and/or retinal cells to decrease offending protein aggregation or inclusion body formation. Such cells may be present in vitro or in a subject (e.g., a patient, such as a human). In certain embodiments, the subject is a subject having, or suspected of having, a protein aggregation-based disorder, particularly Age-Related Macular Degeneration, Inclusion Body Myositis, Myofibrillar Myopathy 2, or Nemaline Myopathy. In some embodiments, the subject is a subject having, or suspected of having, Alzheimer's Disease. In certain embodiments, a chimeric polypeptide of the disclosure is suitable for use in delivering neprilysin to cytoplasm in a subject in need thereof, such as a subject having Age-Related Macular Degeneration, Inclusion Body Myositis, Myofibrillar Myopathy 2, Nemaline Myopathy, or Alzheimer's Disease. In certain embodiments, the subject in need thereof has or is suspected of having Age-Related Macular Degeneration. In certain embodiments, the subject in need thereof has or is suspected of having Myofibrillar Myopathy 2. In certain embodiments, the disclosure provides a method of treating (e.g., improving one or more symptoms of; degrading offending protein aggregation) Age-Related Macular Degeneration. In certain embodiments, the disclosure provides a method of treating (e.g., improving one or more symptoms of; degrading offending protein aggregation) Inclusion Body Myositis. In certain embodiments, the disclosure provides a method of treating (e.g., improving one or more symptoms of; degrading offending protein aggregation) Myofibrillar Myopathy 2. In certain embodiments, the disclosure provides a method of treating (e.g., improving one or more symptoms of; degrading offending protein aggregation) Nemaline Myopathy. Further methods are described herein.

In some embodiments, any of the chimeric polypeptides disclosed herein may be used to degrade offending protein aggregates and inclusion bodies. In some embodiments, the chimeric polypeptides may be used to degrade offending protein aggregates and inclusion bodies in one or more cells of a patient having a disease associated with protein aggregation.

In some embodiments, the chimeric polypeptides of the disclosure may be used to increase degradation of offending protein aggregates and inclusion bodies in a cell. In some embodiments, the cell is a muscle (e.g., cardiac, skeletal, or diaphragm muscle), retinal, or neuronal (e.g., of the brain) cell. In some embodiments, the cell is in a subject having a protein aggregation-based disorder.

In certain embodiments, chimeric polypeptides comprising any of the neprilysin polypeptides disclosed herein can be used to treat Age-Related Macular Degeneration. In certain embodiments, chimeric polypeptides comprising any of the neprilysin polypeptides disclosed herein can be used to treat Inclusion Body Myositis. In certain embodiments, chimeric polypeptides comprising any of the neprilysin polypeptides disclosed herein can be used to treat Myofibrillar Myopathy 2. In certain embodiments, chimeric polypeptides comprising any of the neprilysin polypeptides disclosed herein can be used to treat Nemaline Myopathy. In certain embodiments, the present disclosure provides methods of delivering chimeric polypeptides to cells, including cells in culture (in vitro or ex vivo) and cells in a subject. Delivery to cells in culture, such as healthy cells or cells from a model of disease, have numerous uses. These uses include to identify neprilysin substrates or binding partners, to evaluate localization and/or trafficking (e.g., to cytoplasm, lysosome, and/or autophagic vesicles), to evaluate enzymatic activity under a variety of conditions (e.g., pH), to assess degradation of offending protein aggregates and/or inclusion bodies, and the like. In certain embodiments, chimeric polypeptides of the disclosure can be used as reagents to understand neprilysin activity, localization, and trafficking in healthy or disease contexts.

Delivery to subjects, such as to cells in a subject, has numerous uses. Exemplary therapeutic uses are described below. Moreover, the chimeric polypeptides may be used for diagnostic or research purposes. For example, a chimeric polypeptide of the disclosure may be detectably labeled and administered to a subject, such as an animal model of disease or a patient, and used to image the chimeric polypeptide in the subject's tissues (e.g., localization to muscle, brain and/or retina). Additionally exemplary uses include delivery to cells in a subject, such as to an animal model of disease (e.g., protein aggregation-based disorders). By way of example, chimeric polypeptides of the disclosure may be used as reagents and delivered to animals to understand neprilysin bioactivity, localization and trafficking, protein-protein interactions, enzymatic activity, and impacts on animal physiology in healthy or diseased animals.

In certain embodiments, these methods involve administering to the individual a therapeutically effective amount of a chimeric polypeptide as described above (e.g., a chimeric polypeptide comprising (i) a neprilysin polypeptide and (ii) an internalizing moiety portion). These methods are particularly aimed at therapeutic and prophylactic treatments of animals, and more particularly, humans. With respect to methods for treating protein aggregation-based disorders, the disclosure contemplates all combinations of any of the foregoing aspects and embodiments, as well as combinations with any of the embodiments set forth in the detailed description and examples. Accordingly, chimeric polypeptides of the disclosure are, in certain embodiments, suitable for treating diseases and disorders such as protein aggregation-based disorders. In certain embodiments, the chimeric polypeptide degrades offending protein aggregates and/or inclusion bodies in cells, such as muscle cells (e.g., diaphragm muscle or cardiac muscle cells), retinal cells, and/or neuronal cells, to treat protein aggregation-based disorders in a patient in need thereof.

The present disclosure provides a method of delivering a chimeric polypeptide or nucleic acid construct into a cell via an equilibrative nucleoside transporter (ENT2) pathway, comprising contacting a cell with a chimeric polypeptide or nucleic acid construct. In certain embodiments, the method comprises contacting a cell with a chimeric polypeptide, which chimeric polypeptide comprises a neprilysin polypeptide or bioactive fragment thereof and an internalizing moiety which can mediate transport across a cellular membrane via an ENT2 pathway (and optionally via another ENT transporter, such as ENT3), thereby delivering the chimeric polypeptide into the cell. In certain embodiments, the cell is a muscle cell. The muscle cells targeted using any of the methods disclosed herein may include skeletal (e.g., diaphragm), cardiac or smooth muscle cells. In other embodiments, the chimeric polypeptides are delivered to retinal or neuronal (e.g., brain) cells.

The present disclosure also provides a method of delivering a chimeric polypeptide or nucleic acid construct into a cell via a pathway that allows access to cells other than muscle cells. Other cell types that could be targeted using any of the methods disclosed herein include, for example, liver cells, neurons (e.g., of the brain), epithelial cells, uterine cells, and kidney cells.

In certain embodiments, the internalizing moiety is an antibody or antigen binding fragment, such as an antibody or antigen binding fragment that binds DNA. In certain embodiments, the internalizing moiety is an antibody, such as a full-length antibody or a Fab. In certain embodiments, the full-length antibody or Fab comprises one or more substitutions, relative to a native immunoglobulin constant region, such as to decrease effector function.

Age-Related Macular Degeneration (AMD) is a major cause of vision impairment in as many as 11 million people in the United States, and is expected to double to nearly 22 million by 2050. It is the leading cause of vision loss in people 50 years or older due to damage to the macular, a small spot near the center of the retinal required for sharp central vision. Age is the primary risk factor for AMD, with it typically occurring in patients over age 60; diagnosis typically occurs between 54 to 77 years of age. Additional risk factors include smoking (doubles the risk), race (Caucasians are more at risk than African-Americans or Hispanics/Latinos), and family history/genetics (20 genes have been correlated with a risk of developing AMD). The global cost of visual impairment due to AMD is estimated to be $343 billion, including $255 billion in direct health care costs.

Dry (GA) AMD is characterized by the gradual breakdown of the light-sensitive cells in the macula that convey images to the brain, and of the supporting tissue beneath the macula. Additional features of dry AMD include: drusen is present in or under the regional pigment epithelium (RPE); irregularities occur in the pigmentation of the RPE, and geographic atrophy (GA) is present. Normally, RPE is involved in recycling lipid-rich photoreceptor outer segments. However, impairment of normal physiological function in RPE cells can lead to the formation of cytotoxic quantities of lipofuscin, which are lipid rich fluorescent pigment granules that manifest as a fluorescent signal in fundus autofluorescence images. Preceding photoreceptor loss in GA, a number of early changes occur in the outer retina, including: thickening of Bruch's membrane and deposition of waste products; lipofuscin accumulation in the RPE; RPE hypopigmentation; and/or drusen formation beneath the RPE cell monolayer. In some aspects, drusen contain pro-inflammatory molecules that damage the RPE by inflammation. In addition, oxidative stress can contribute to AMD. For instance, CFH Y402H can no longer bind to malondialdehyde to clear it, leading to further oxidative modification of proteins and lipids in the area. See van Lookeren Campagne et al. J. Pathol (2014) 232:151-164. With large drusen and hyperpigmentation, early/intermediate AMD can progress to GA within 5-6 years and hypopigmentation within 2.5 years. Dry AMD accounts for 80%-90% of all AMD cases, causes ˜20% of all legally-blind cases, and currently has no treatment. Associated genetic mutations include: HMCN1, CFHR3, CFHR1, APOE (AMD1); ABCA4 (AMD2); FBLN5 (AMD3); and CFH (AMD4; specifically Y402H). OMIM entries include 603075 (polymorphic AMD), 153800 (ABCA4 gene), 608895 (FBLN5), and 610698 (CFH).

Inclusion Body Myositis (IBM) is characterized by the presence of inclusion bodies in muscle cells that leads to muscle inflammation (e.g., the presence of insoluble protein aggregates and a pathological autoimmune response against skeletal muscle tissue). IBM primarily affects men, although women can also be affected (ratio ˜2:1). IBM typically onsets after the age of 50, and exhibits a slow progression. The prevalence of IBM is estimated between 0.3 and 13.9 per 100,000. The underlying cause of IBM is unclear, but certain observations include: myositis is seen after viral infections like HIV and Coxsackie B; some drugs appear to be linked to myositis development; genetic IBM can be either dominant or recessive depending on the aggregating protein; and there is conflicting evidence of causation by specific protein aggregates such as β-amyloid or tau. See Alfano et al. DovePress (2015) 11: 1459-1467.

IBM typically presents with muscle weakness with characteristic pattern in quadriceps and finger flexors, and is often misdiagnosed as polymyositis or ALS. Muscle weakness progresses throughout the body over time, with patients developing requirement for wheelchair and assistance with daily tasks after 10-15 years. Typically, the heart muscle is spared from weakness.

Myofibrillar Myopathy 2 (MFM2) is characterized by adult onset of progressive muscle weakness affecting both the proximal and distal muscles and associated with respiratory insufficiency, cardiomyopathy, and cataracts. MFM2 often presents with proximal and distal limb muscle weakness often associated with neck, trunk, and velopharynx muscle involvement. In some aspects, there are signs of cardiomyopathy. Numerous desmin-reactive aggregates in the muscle fibers may be present, and intrasarcoplasmic accumulation of dense granulofilamentous material may be identified using electron microscopy.

MFM2 may be caused by an autosomal dominant mutation in the CRYAB gene. There may be phenotypic variability within patient populations, but all possess mutations, either homozygous or heterozygous, in the CRYAB gene. Other Desminopathies include mutations in the desmin, dystrophin, or myotilin genes and all of these mutations lead to protein aggregation. CryAB(R120G) causes desmin-related myopathy and is associated with decreased neprilysin activity. In some aspects, neprilysin activity increased with exercise, which in turn decreased pathogenic aggregate formation (Maloyan et al., PNAS (2007) 104(14): 5995-6000). MFM2 may include abnormal expanses of homogeneous material and myofibrillar disintegration involving the Z discs, which may indicate protein aggregation, and likely aggregation-related injury to fibers. In some aspects, small autophagic vacuoles are also present in muscle biopsies.

Nemaline Myopathy (NM) is characterized by weakness and hypotonia with depressed or absent deep tendon reflexes. In some aspects, muscle weakness, hypotonia and reduced or absent reflexes is present; with the most severe weakness occurring in muscles of the face, neck and proximal muscles. In addition, the presence of fine, thread-like or rod-like structures called “nemaline bodies” are present when muscle biopsies are viewed under the microscope. Onset of NM ranges from a severe congenital-onset (at birth) form that is usually lethal in the first few months of life, through to less severe forms with onset in childhood or adulthood. There are 6 different clinical presentations: Typical Congenital, 50%, <1 year-olds; Severe Congenital, 16%, <1 yo; Intermediate Congenital, 20%, <1 yo; Childhood-Onset, 13%, 10-20 yo; and Adult-Onset, 4%, 20-50 yo.

NM prevalence has been examined in multiple studies, one in Finland and one with American Ashkenazi Jewish population, which estimate 1:50,000 patient prevalence. Another study performed in the Amish community, estimated patient prevalence to be 1:500. To date, 10 genes, which can be autosomal dominant or recessive, have been implicated in nemaline rod formation including: Slow α-tropomyosin (TPM3) (Wallgren-Pettersson, et al. Neuromuscul Disord. (2003) 13(6):501-507); NEB; ACTA1 (Sparrow et al., Neuromuscul Disord. (2003) 13(7-8):519-531.); Muscle troponin T gene (TNNT1); and β-tropomyosin gene (TPM2). NEB accounts for about 50% of the cases, while ACTA1 accounts for about 15-25%, with the remaining genes being more rare. In addition, these are proteins that play an essential role in the normal structure and function of the contractile apparatus of skeletal muscle. These proteins together form thin filaments in the sarcomere and play a role in the formation and contractile function of skeletal muscle fibers. Therefore, if these proteins are deficient or defective, the strength of muscle contraction and in some cases the development of normal muscle structure is impaired. It is currently unclear whether nemaline rods themselves cause NM, or whether the lack of normal protein function causes the disease.

The terms “treatment”, “treating”, and the like are used herein to generally mean obtaining a desired pharmacologic and/or physiologic effect. “Treating” a condition or disease refers to curing as well as ameliorating at least one symptom of the condition or disease, and includes administration of a composition which reduces the frequency of, or delays the onset of, symptoms of a medical condition in a subject in need relative to a subject which does not receive the composition. “Treatment” as used herein covers any treatment of a disease or condition of a mammal, particularly a human, and includes: (a) preventing symptoms of the disease or condition from occurring in a subject which may be predisposed to the disease or condition but has not yet begun experiencing symptoms; (b) inhibiting the disease or condition (e.g., arresting its development); or (c) relieving the disease or condition (e.g., causing regression of the disease or condition, providing improvement in one or more symptoms). For example, “treatment” of protein aggregation-based disorders is contemplated and encompasses a complete reversal or cure of the disease, or any range of improvement in symptoms and/or adverse effects attributable to the disease.

The population of subjects treated by the method of the disclosure includes subjects suffering from the undesirable condition or disease, as well as subjects at risk for development of the condition or disease.

In certain embodiments, the disclosure provides methods of delivering neprilysin activity to cells, such as muscle and/or retinal and/or neuron cells of a subject having a protein aggregation-based disorder comprising administering a chimeric polypeptide of the disclosure or a composition comprising a chimeric polypeptide of the disclosure formulated with one or more pharmaceutically acceptable carriers and/or excipients.

By the term “therapeutically effective dose” is meant a dose that produces the desired effect for which it is administered. The exact dose will depend on the purpose of the treatment, and will be ascertainable by one skilled in the art using known techniques (see, e.g., Lloyd (1999) The Art, Science and Technology of Pharmaceutical Compounding).

In certain embodiments, administration of a chimeric polypeptide of the disclosure is via any one of the routes of administration described herein, such as subcutaneous, intravenous, or via the hepatic portal vein. In other words, the disclosure contemplates methods of delivery by administering via any such route of administration.

In certain embodiments, the method results in delivery of greater neprilysin activity to the cytoplasm, in comparison, to that following delivery of a neprilysin polypeptide that is not conjugated to an internalizing moiety and/or in comparison to that of a neprilysin polypeptide conjugated to a different internalizing moiety.

In certain embodiments, one or more chimeric polypeptides of the present disclosure can be administered, together (simultaneously) or at different times (sequentially). In addition, chimeric polypeptides of the present disclosure can be administered alone or in combination with one or more additional compounds or therapies for treating protein aggregation-based disorders. For example, one or more chimeric polypeptides can be co-administered in conjunction with one or more other therapeutic compounds. When co-administration is indicated, the combination therapy may encompass simultaneous or alternating administration. In addition, the combination may encompass acute or chronic administration. Optionally, the chimeric polypeptide of the present disclosure and additional compounds act in an additive or synergistic manner for treating protein aggregation-based disorders. Additional compounds to be used in combination therapies include, but are not limited to, small molecules, polypeptides, antibodies, antisense oligonucleotides, and siRNA molecules. Depending on the nature of the combinatory therapy, administration of the chimeric polypeptides of the disclosure may be continued while the other therapy is being administered and/or thereafter. Administration of the chimeric polypeptides may be made in a single dose, or in multiple doses. In some instances, administration of the chimeric polypeptides is commenced at least several days prior to the other therapy, while in other instances, administration is begun either immediately before or at the time of the administration of the other therapy.

In another example of combination therapy, one or more chimeric polypeptides of the disclosure can be used as part of a therapeutic regimen combined with one or more additional treatment modalities. By way of example, such other treatment modalities include, but are not limited to, dietary therapy, occupational therapy, physical therapy, ventilator supportive therapy, massage, acupuncture, acupressure, mobility aids, assistance animals, and the like.

Note that although the chimeric polypeptides described herein can be used in combination with other therapies, in certain embodiments, a chimeric polypeptide is provided as the sole form of therapy. Regardless of whether administrated alone or in combination with other medications or therapeutic regiments, the dosage, frequency, route of administration, and timing of administration of the chimeric polypeptides is determined by a physician based on the condition and needs of the patient. The disclosure contemplates that a method may comprise administration at a dose and on a dosing schedule, such as administration at specified intervals over a period of time. In such cases, each dose contributes to efficacy, and is thus effective, although improvement in symptoms may only be observed after administration of multiple doses.

Chimeric polypeptides of the disclosure have numerous uses, including in vitro and in vivo uses. In vivo uses include not only therapeutic uses but also diagnostic and research uses in, for example, any of the foregoing animal models. By way of example, chimeric polypeptides of the disclosure may be used as research reagents and delivered to animals to understand neprilysin bioactivity, localization and trafficking, protein-protein interactions, enzymatic activity, and impacts on animal physiology in healthy or diseases animals.

Chimeric polypeptides may also be used in vitro to evaluate, for example, neprilysin bioactivity, localization and trafficking, protein-protein interactions, and enzymatic activity in cells in culture, including healthy and neprilysin deficient cells in culture. The disclosure contemplates that chimeric polypeptides of the disclosure may be used to deliver neprilysin to cytoplasm, lysosome, and/or autophagic vesicles of cells, including cells in culture.

The disclosure contemplates that any of the methods described herein may be carried out by administering or contacting cells with a chimeric polypeptide of the disclosure and/or a composition of the disclosure (e.g., a composition comprising a chimeric polypeptide of the disclosure formulated with one or more pharmaceutically acceptable carriers and/or excipients).

### VI. Gene Therapy

Conventional viral and non-viral based gene transfer methods can be used to introduce nucleic acids encoding polypeptides of neprilysin (e.g., a neprilysin ectodomain) and or chimeric polypeptides comprising neprilysin in mammalian cells or target tissues. Such methods can be used to administer nucleic acids encoding polypeptides of the disclosure (e.g., neprilysin including variants thereof, and include chimeric polypeptides) to cells in vitro. The disclosure contemplates that gene transfer methods may be used to deliver nucleic acid encoding any of the chimeric polypeptides of the disclosure or neprilysin polypeptides. In some embodiments, the nucleic acids encoding neprilysin are administered for in vivo or ex vivo gene therapy uses. In other embodiments, gene delivery techniques are used to study the activity of chimeric polypeptides or neprilysin polypeptide or to study protein aggregation-based disorders in cell based or animal models, such as to evaluate cell trafficking, enzyme activity, and protein-protein interactions following delivery to healthy or diseased cells and tissues. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell. Such methods are well known in the art.

Methods of non-viral delivery of nucleic acids encoding engineered polypeptides of the disclosure include lipofection, microinjection, biolistics, virosomes, liposomes, immunoliposomes, polycation or lipid:nucleic acid conjugates, naked DNA, artificial virions, and agent-enhanced uptake of DNA. Lipofection methods and lipofection reagents are well known in the art (e.g., Transfectam™ and Lipofectin™). Cationic and neutral lipids that are suitable for efficient receptor-recognition lipofection of polynucleotides include those of Felgner, WO 91/17424, WO 91/16024. Delivery can be to cells (ex vivo administration) or target tissues (in vivo administration). The preparation of lipid:nucleic acid complexes, including targeted liposomes such as immunolipid complexes, is well known to one of skill in the art.

The use of RNA or DNA viral based systems for the delivery of nucleic acids encoding neprilysin or its variants take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of polypeptides of the disclosure could include retroviral, lentivirus, adenoviral, adeno-associated and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SW), human immuno deficiency virus (HIV), and combinations thereof, all of which are well known in the art.

In applications where transient expression of the polypeptides of the disclosure is preferred, adenoviral based systems are typically used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures. Construction of recombinant AAV vectors are described in a number of publications, including U.S. Pat. No. 5,173,414; Tratschin et al., Mol. Cell. Biol. 5:3251-3260 (1985); Tratschin, et al.; Mol. Cell. Biol. 4:2072-2081 (1984); Hermonat & Muzyczka, PNAS 81:6466-6470 (1984); and Samulski et al., J. Virol. 63:03822-3828 (1989).

Recombinant adeno-associated virus vectors (rAAV) are a promising alternative gene delivery systems based on the defective and nonpathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system.

Replication-deficient recombinant adenoviral vectors (Ad) can be engineered such that a transgene replaces the Ad E1a, E1b, and E3 genes; subsequently the replication defector vector is propagated in human 293 cells that supply deleted gene function in trans. Ad vectors can transduce multiple types of tissues in vivo, including nondividing, differentiated cells such as those found in the liver, kidney and muscle system tissues. Conventional Ad vectors have a large carrying capacity.

Packaging cells are used to form virus particles that are capable of infecting a host cell. Such cells include 293 cells, which package adenovirus, and 42 cells or PA317 cells, which package retrovirus. Viral vectors used in gene therapy are usually generated by producer cell line that packages a nucleic acid vector into a viral particle. The vectors typically contain the minimal viral sequences required for packaging and subsequent integration into a host, other viral sequences being replaced by an expression cassette for the protein to be expressed. The missing viral functions are supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In many gene therapy applications, it is desirable that the gene therapy vector be delivered with a high degree of specificity to a particular tissue type. A viral vector is typically modified to have specificity for a given cell type by expressing a ligand as a fusion protein with a viral coat protein on the viruses outer surface. The ligand is chosen to have affinity for a receptor known to be present on the cell type of interest. This principle can be extended to other pairs of virus expressing a ligand fusion protein and target cell expressing a receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences thought to favor uptake by specific target cells, such as muscle cells.

Gene therapy vectors can be delivered in vivo by administration to an individual patient, by systemic administration (e.g., intravenous, intraperitoneal, intramuscular, subdermal, or intracranial infusion) or topical application. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

Ex vivo cell transfection for diagnostics, research, or for gene therapy (e.g., via re-infusion of the transfected cells into the host organism) is well known to those of skill in the art. For example, cells are isolated from the subject organism, transfected with a nucleic acid (gene or cDNA) encoding, e.g., neprilysin or its variants, and re-infused back into the subject organism (e.g., patient). Various cell types suitable for ex vivo transfection are well known to those of skill in the art.

In certain embodiments, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Stem cells are isolated for transduction and differentiation using known methods.

Vectors (e.g., retroviruses, adenoviruses, liposomes, etc.) containing therapeutic nucleic acids can also be administered directly to the organism for transduction of cells in vivo. Alternatively, naked DNA can be administered. Administration is by any of the routes normally used for introducing a molecule into ultimate contact with blood or tissue cells. Suitable methods of administering such nucleic acids are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions of the present disclosure, as described herein.

### VII. Methods of Administration

Various delivery systems are known and can be used to administer the chimeric polypeptides of the disclosure. Any such methods may be used to administer any of the chimeric polypeptides described herein. The disclosure contemplates than any of the methods of administration disclosed herein may be used to deliver any of the chimeric polypeptides of the disclosure in the context of any of the methods described herein (e.g., methods of treatment; methods of reducing offending protein aggregates and inclusion bodies).

Methods of introduction can be enteral or parenteral, including but not limited to, intradermal, intramuscular, intraperitoneal, intramyocardial, intravenous, subcutaneous, pulmonary, intranasal, intraocular, epidural, intrathecal, intracranial, intraventricular and oral routes. The chimeric polypeptides may be administered by any convenient route, for example, by infusion or bolus injection, by absorption through epithelial or mucocutaneous linings (e.g., oral mucosa, rectal and intestinal mucosa, etc.) and may be administered together with other biologically active agents. Administration can be systemic or local.

In certain embodiments, the chimeric polypeptide is administered intravenously.

In certain embodiments, it may be desirable to administer the chimeric polypeptides of the disclosure locally to the area in need of treatment (e.g., muscle); this may be achieved, for example, and not by way of limitation, by local infusion during surgery, by means of a catheter, or by means of an implant, the implant being of a porous, non-porous, or gelatinous material, including membranes, such as sialastic membranes, fibers, or commercial skin substitutes.

In another embodiment, such local administration can be to all or a portion of the heart. For example, administration can be by intrapericardial or intramyocardial administration. Similarly, administration to cardiac tissue can be achieved using a catheter, wire, and the like intended for delivery of agents to various regions of the heart.

Note that the disclosure contemplates methods in which chimeric polypeptides are administered, at the same or different times, via one than one route of administration. For example, the disclosure contemplates a regimen in which chimeric polypeptides are administered systemically, such as by intravenous infusion, in combination with local administration via the hepatic portal vein.

In other embodiments, the chimeric polypeptides of the disclosure can be delivered in a vesicle, in particular, a liposome (see Langer, 1990, Science 249:1527-1533). In yet another embodiment, the chimeric polypeptides of the disclosure can be delivered in a controlled release system. In another embodiment, a pump may be used (see Langer, 1990, supra). In another embodiment, polymeric materials can be used (see Howard et al., 1989, J. Neurosurg. 71:105). In certain specific embodiments, the chimeric polypeptides of the disclosure can be delivered intravenously.

In certain embodiments, the chimeric polypeptides are administered by intravenous infusion. In certain embodiments, the chimeric polypeptides are infused over a period of at least 10, at least 15, at least 20, or at least 30 minutes. In other embodiments, the chimeric polypeptides are infused over a period of at least 60, 90, or 120 minutes. Regardless of the infusion period, the disclosure contemplates that each infusion is part of an overall treatment plan where chimeric polypeptide is administered according to a regular schedule (e.g., weekly, monthly, etc.).

The foregoing applies to any of the chimeric polypeptides, compositions, and methods described herein. The disclosure specifically contemplates any combination of the features of such chimeric polypeptides, compositions, and methods (alone or in combination) with the features described for the various pharmaceutical compositions and route of administration described in this section.

### VIII. Pharmaceutical Compositions

In certain embodiments, the subject chimeric polypeptides for use in any of the methods disclosed herein are formulated with a pharmaceutically acceptable carrier (e.g., formulated with one or more pharmaceutically acceptable carriers and/or excipients). One or more chimeric polypeptides can be administered alone or as a component of a pharmaceutical formulation (composition). Any of the chimeric polypeptides described herein may be formulated, as described herein, and any such compositions (e.g., pharmaceutical compositions, or preparations, or formulations) may be used in any of the methods described herein. In other embodiments, the composition comprises a chimeric polypeptide comprising a neprilysin polypeptide. The chimeric polypeptides may be formulated for administration in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the subject chimeric polypeptides include, for example, those suitable for oral, nasal, topical, parenteral, rectal, and/or intravaginal administration. The formulations may conveniently be presented in unit dosage form and may be prepared by any methods well known in the art of pharmacy. The amount of active ingredient which can be combined with a carrier material to produce a single dosage form will vary depending upon the host being treated and the particular mode of administration. The amount of active ingredient which can be combined with a carrier material to produce a single dosage form will generally be that amount of the compound which produces a therapeutic effect.

In certain embodiments, methods of preparing these formulations or compositions include combining another type of therapeutic agents and a carrier and, optionally, one or more accessory ingredients. In general, the formulations can be prepared with a liquid carrier, or a finely divided solid carrier, or both, and then, if necessary, shaping the product.

In certain embodiments, any of the pharmaceutical compositions described herein comprise concentrated amounts of any of the chimeric polypeptides described herein. In some embodiments, the compositions have 50%, 100%, 150%, 200%, 250%, 300%, 350% or 400% more concentrated levels of the chimeric polypeptide as compared to the levels of chimeric polypeptide originally purified from the cells producing the chimeric polypeptide. In some embodiments, the concentration of the chimeric polypeptide is at least 2, 3, 4, 5, 6, 7, 8, 9, 10, 11, 12, 13, 14, 15, 16, 17, 18, 19, or 20 mg/ml. In some embodiments, the concentration of the chimeric polypeptide is at least 10 mg/ml or greater. In some embodiments, the concentration of the chimeric polypeptide is at least 15 mg/ml or greater. In some embodiments, the concentration of the chimeric polypeptide is at least 20 mg/ml or greater. In some embodiments, the concentration of the chimeric polypeptide is at least 30 mg/ml or greater. In some embodiments, the concentration of the chimeric polypeptide is at least 50 mg/ml or greater. In some embodiments, the concentration of the chimeric polypeptide is at least 70 mg/ml or greater. In some embodiments, the concentration of the chimeric polypeptide is at least 90 mg/ml or greater. In some embodiments, the concentration of the chimeric polypeptide is at least 110 mg/ml or greater. In some embodiments, the concentration of the chimeric polypeptide is 10-50 mg/ml, 10-40 mg/ml, 10-30 mg/ml, 10-25 mg/ml, 10-20 mg/ml. 20-50 mg/ml, 50-70 mg/ml, 70-90 mg/ml or 90-110 mg/ml. In some embodiments, any of the compositions described herein preserve at least 80%, 90%, 95% or 100% biological activity (as defined herein) for at least 24 hours, 2 days, 4 days, 1 week, 2 weeks or 1 month when stored in a pharmaceutically acceptable formulation at 4° C. In some embodiments of any of the foregoing, the chimeric polypeptide portion of the composition is substantially pure, as described herein (e.g., greater than 85% of the neprilysin present is in association or interconnected with an internalizing moiety).

Formulations for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a subject chimeric polypeptide therapeutic agent as an active ingredient. Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

In solid dosage forms for oral administration (capsules, tablets, pills, dragees, powders, granules, and the like), one or more chimeric polypeptide therapeutic agents of the present disclosure may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like. Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

In certain embodiments, methods of the disclosure include topical administration, either to skin or to mucosal membranes such as those on the cervix and vagina. The topical formulations may further include one or more of the wide variety of agents known to be effective as skin or stratum corneum penetration enhancers. Examples of these are 2-pyrrolidone, N-methyl-2-pyrrolidone, dimethylacetamide, dimethylformamide, propylene glycol, methyl or isopropyl alcohol, dimethyl sulfoxide, and azone. Additional agents may further be included to make the formulation cosmetically acceptable. Examples of these are fats, waxes, oils, dyes, fragrances, preservatives, stabilizers, and surface active agents.

Keratolytic agents such as those known in the art may also be included. Examples are salicylic acid and sulfur. Dosage forms for the topical or transdermal administration include powders, sprays, ointments, pastes, creams, lotions, gels, solutions, patches, and inhalants. The subject polypeptide therapeutic agents may be mixed under sterile conditions with a pharmaceutically acceptable carrier, and with any preservatives, buffers (e.g., HEPES buffer), or propellants which may be required. The ointments, pastes, creams and gels may contain, in addition to a subject chimeric polypeptide agent, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays can contain, in addition to a subject chimeric polypeptides, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

Pharmaceutical compositions suitable for parenteral administration may comprise one or more chimeric polypeptides in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers (e.g., HEPES buffer), bacteriostats, solutes which render the formulation isotonic with the blood of the intended recipient or suspending or thickening agents. Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

Injectable depot forms are made by forming microencapsule matrices of one or more polypeptide therapeutic agents in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

In a preferred embodiment, the chimeric polypeptides of the present disclosure are formulated in accordance with routine procedures as a pharmaceutical composition adapted for intravenous administration to human beings. Where necessary, the composition may also include a solubilizing agent and a local anesthetic such as lidocaine to ease pain at the site of the injection. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In another embodiment, the chimeric polypeptides of the present disclosure are formulated for subcutaneous administration to human beings.

In certain embodiments, the chimeric polypeptides of the present disclosure are formulated for intrathecal, intracranial and/or intraventricular delivery. In certain embodiments, a chimeric polypeptide of the disclosure for use in treating protein aggregation-based disorders or for use in degrading offending protein aggregates and/or inclusion bodies, such as in a subject having a protein aggregation-based disorder, is formulated for intrathecal, intracranial and/or intraventricular delivery. In certain embodiments, a method of the disclosure, such as a method of treating protein aggregation-based disorders or for degrading offending protein aggregates and/or inclusion bodies in cells, comprising delivering a chimeric polypeptide of the disclosure intrathecally, intracranially and/or intraventricularly.

In certain embodiments, the chimeric polypeptides of the present disclosure are formulated for delivery to the heart, such as for intramyocardial or intrapericaridal delivery.

Note that, in certain embodiments, a particular formulation is suitable for use in the context of delivery via more than one route. However, in other embodiments, a formulation is suitable for use in the context of one route of delivery, but is not suitable for use in the context of a second route of delivery.

The amount of the chimeric polypeptides of the disclosure which will be effective in the treatment of a tissue-related condition or disease (e.g., protein aggregation-based disorders) can be determined by standard clinical techniques. In addition, in vitro assays may optionally be employed to help identify optimal dosage ranges. The precise dose to be employed in the formulation will also depend on the route of administration, and the seriousness of the condition, and should be decided according to the judgment of the practitioner and each subject's circumstances. However, suitable dosage ranges for intravenous administration are generally about 20-5000 micrograms of the active chimeric polypeptide per kilogram body weight. Suitable dosage ranges for intranasal administration are generally about 0.01 μg/kg body weight to 1 mg/kg body weight. Effective doses may be extrapolated from dose-response curves derived from in vitro or animal model test systems.

In certain embodiments, compositions of the disclosure, including pharmaceutical preparations, are non-pyrogenic. In other words, in certain embodiments, the compositions are substantially pyrogen free. In one embodiment the formulations of the disclosure are pyrogen-free formulations which are substantially free of endotoxins and/or related pyrogenic substances. Endotoxins include toxins that are confined inside a microorganism and are released only when the microorganisms are broken down or die. Pyrogenic substances also include fever-inducing, thermostable substances (glycoproteins) from the outer membrane of bacteria and other microorganisms. Both of these substances can cause fever, hypotension and shock if administered to humans. Due to the potential harmful effects, even low amounts of endotoxins must be removed from intravenously administered pharmaceutical drug solutions. The Food & Drug Administration (“FDA”) has set an upper limit of 5 endotoxin units (EU) per dose per kilogram body weight in a single one hour period for intravenous drug applications (The United States Pharmacopeial Convention, Pharmacopeial Forum 26 (1):223 (2000)). When therapeutic proteins are administered in relatively large dosages and/or over an extended period of time (e.g., such as for the patient's entire life), even small amounts of harmful and dangerous endotoxin could be dangerous. In certain specific embodiments, the endotoxin and pyrogen levels in the composition are less then 10 EU/mg, or less then 5 EU/mg, or less then 1 EU/mg, or less then 0.1 EU/mg, or less then 0.01 EU/mg, or less then 0.001 EU/mg.

In some embodiments, the disclosure provides a composition, such as a pharmaceutical composition comprising a chimeric polypeptide of the disclosure formulated with one or more pharmaceutically acceptable carriers and/or excipients. Such compositions include compositions comprising any of the internalizing moiety portions, described herein, and a neprilysin portion, as described herein. For example, the disclosure provides compositions comprising a neprilysin-containing chimeric polypeptide. In certain embodiments, any of the compositions described herein may be based on any of the neprilysin portions and/or internalizing moiety portions described herein. Moreover, any such compositions may be described based on any of the structural and/or functional features described herein. Any such compositions may be used in any of the methods described herein, such as administered to cells and/or to subjects in need of treatment, such as administered to cells and/or to subjects having protein aggregation-based disorders. Any such compositions may be used to deliver neprilysin activity into cells, such as into muscle and/or retinal cells in a patient in need thereof (e.g., a patient having protein aggregation-based disorders).

Such compositions, including any of the compositions described herein, may be provided, for example, in a bottle or syringe and stored prior to administration.

The foregoing applies to any of the chimeric polypeptides, compositions, and methods described herein. The disclosure specifically contemplates any combination of the features of such chimeric polypeptides, compositions, and methods (alone or in combination) with the features described for the various pharmaceutical compositions and route of administration described in this section.

### IX. Kits

In certain embodiments, the disclosure also provides a pharmaceutical package or kit comprising one or more containers filled with at least one chimeric polypeptide of the disclosure. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

In certain embodiments, the kit includes additional materials to facilitate delivery of the subject chimeric polypeptides. For example, the kit may include one or more of a catheter, tubing, infusion bag, syringe, and the like. In certain embodiments, the chimeric polypeptide is packaged in a lyophilized form, and the kit includes at least two containers: a container comprising the lyophilized chimeric polypeptide and a container comprising a suitable amount of water, buffer (e.g., HEPES buffer), or other liquid suitable for reconstituting the lyophilized material.

The foregoing applies to any of the chimeric polypeptides, compositions, and methods described herein. The disclosure specifically contemplates any combination of the features of such chimeric polypeptides, compositions, and methods (alone or in combination) with the features described for the various kits described in this section.

### Exemplification

The disclosure now being generally described, it will be more readily understood by reference to the following examples, which are included merely for purposes of illustration of certain aspects and embodiments of the present disclosure, and are not intended to limit the disclosure. For example, the particular constructs and experimental design disclosed herein represent exemplary tools and methods for validating proper function. As such, it will be readily apparent that any of the disclosed specific constructs and experimental plan can be substituted within the scope of the present disclosure.

A variety of human diseases arise as a result of offending protein aggregation or inclusion body (IB) formation, from aging disorders such as Age-Related Macular Degeneration (AMD) to rare genetic disorders such as Desminopathies (e.g., MFM2). IBs can impede normal protein function by removing functional proteins from their active location, and they can trigger cytotoxic protein interactions. Thus, IB clearance is an attractive therapeutic objective toward preventing disease progression, and may allow the body's natural clearance mechanisms to catch up for a return to a pre-disease state.

Toward this end, an antibody-enzyme fusion with neprilysin (Fab-NEP) thought to be capable of uniquely penetrating cells and degrading offending IBs has been generated. The humanized Fab portion has provided intracellular delivery for a variety of tested proteins. Neprilysin is a ubiquitously-expressed zinc-dependent endopeptidase that has been shown to degrade a variety of substrates, including β-amyloid. Here, it is demonstrated that Fab-NEP activity is equal to wild-type Neprilysin activity in ex vivo degradation assays. The ability of Fab-NEP to degrade a range of relevant protein aggregates in vitro in AMD and MFM2 models is currently under investigation. As there are no current treatments for AMD or MFM2 that clear drusen or muscle protein aggregates, Fab-NEP has the potential to be a novel treatment approach to a class of age-related and rare diseases with high unmet need.

### Example 1: Chemical Conjugation of Fab-Neprilysin

Chimeric polypeptides comprising a neprilysin polypeptide portion (e.g., neprilysin ectodomain) and an internalizing moiety portion were made recombinantly in HEK-293 cells. In alternative embodiments, chimeric polypeptides comprising a neprilysin polypeptide portion (e.g., neprilysin ectodomain) and an internalizing moiety portion are made recombinantly in E. coli cells. A neprilysin polypeptide comprising a neprilysin ectodomain polypeptide (e.g., a polypeptide having the amino acid sequence of SEQ ID NO: 2) was fused to a Fab of a humanized 3E10 antibody comprising the heaving chain variable domain set forth in SEQ ID NO: 6. Specifically, a neprilysin ectodomain polypeptide having the amino acid sequence of SEQ ID NO: 2 was fused to the C-terminus of the heavy chain constant region of a humanized 3E10 Fab fragment by means of a linker having the amino acid sequence of SEQ ID NO: 5 to generate a fusion polypeptide having the amino acid sequence of SEQ ID NO: 29. The light chain comprises the amino acid sequence of SEQ ID NO: 7. The resulting “Fab-neprilysin” comprising both the heavy chain and light chains is referred to in the experimental designs described below.

This Fab was made by expressing a vector encoding the light chain and a vector encoding the heavy chain-neprilysin fusion in a HEK-293 cell line. Although two separate vectors were used, a single vector encoding both the heavy and light chain could also have been employed.

A nucleotide sequence encoding the recombinant heavy chain (SEQ ID NO: 29) and a nucleotide sequence encoding the light chain (SEQ ID NO: 7) was codon optimized for mammalian cell expression and cloned into the pTT5 vector using standard methods. Low endotoxin, giga-prep scale production of the expression plasmid encoding the sequence of SEQ ID NO: 29 and the expression plasmid encoding the sequence of SEQ ID NO: 7 resulted in 7.0 mg of each plasmid DNA (each, a vector). HEK-293 cells were then transfected with these two plasmids in a manner summarized below.

Twenty, 1 L cultures of 293 cells (initial density of 2.6×10⁶ cells/mL) in 2 L shake flasks were transfected with 1 mg total (1:1 ratio HC:LC) of plasmid DNA/L culture using PolyPlus linear Q-PEI at a 1:1.5 (w/v) DNA:PEI ratio. Culture parameters were monitored using a ViCell XR for density and viability. The culture media was F17 supplemented with 0.1% Pluronic F-68, 4 mM GlutaMAX, 25 μg/mL G418. Cells were maintained at a density between 0.5-5×10⁶ cells/mL in shake flasks. The flasks were incubated at 37° C. in a humidified 5% CO₂ environment with shaking at 135 rpm. Cultures were harvested 6 days post-transfection via centrifugation for 5 minutes at 1000×g. The conditioned culture supernatant was clarified by centrifugation for 30 minutes at 9300×g.

Fab-neprilysin was purified from the HEK-293 cells using a CaptureSelect IgG-CH1 affinity matrix (Life Technologies, #194320001). The CaptureSelect IgG-CH1 affinity resin was equilibrated in buffer A (1×PBS (2.7 mM KCl, 1.7 mM KH₂PO₄, 136 mM NaCl, 10.1 mM Na₂HPO₄), pH 7.2 (23° C.)). Fab-neprilysin from 20 L of exhausted supernatant was batch bound with the CaptureSelect IgG-CH1 affinity resin (40 mL bed volume) at 4° C. overnight with stirring. The resin was collected in a 5 cm diameter Econo-column and washed with approximately 15 column volumes (CV) of Buffer A, 15 CV of Buffer B (1×PBS, 500 mM NaCl, pH 7.2 (23° C.)) and 15 CV buffer A. The resin-bound fusion protein was eluted with ˜4 CV of Buffer C (30 mM NaOAc, pH 3.5-3.6 (23° C.)) followed by ˜4 CV of Buffer D (100 mM Glycine, pH 2.7 (23° C.)) collecting the protein in 10 mL fractions diluted in 1/10th volume Buffer E (3M NaAcetate, ˜pH 9.0 (23° C.)) to neutralize. To minimize the elution volume, elution was paused for several minutes between each fraction collected. Fractions were analyzed by A₂₈₀ prior to pooling fractions 7-25. Select fractions were analyzed by SDS PAGE. Fab-neprilysin remained in the non-bound pool from the first affinity chromatography pass. The above procedure was repeated to capture remaining Fab-neprilysin. The affinity pools were combined prior to dialysis.

The combined CaptureSelect IgG-CH1 affinity pool (250 mL) was dialyzed against 3×4 L of dialysis buffer (20 mM Histidine, 150 mM NaCl, pH 6.5 (23° C.)) at 4° C. The dialyzed pool was concentrated to −10 mg/mL using a VivaCell 100 (10K MWCO, PES membrane) centrifugal device prior to final analysis and storage at −80° C. Select fractions were analyzed by SDS-PAGE and by size exclusion chromotography, where it was confirmed that the Fab-alpha-amylase was being produced and successfully purified (data not shown).

In alternative embodiments, a protein comprising a full-length humanized 3E10 antibody and the neprilysin protein may be generated. Other chimeric proteins of the disclosure may be, for example, similarly made, and any such proteins may be used in any of the methods described herein.

In alternative embodiments, chimeric polypeptides comprising a neprilysin polypeptide portion (e.g., neprilysin ectodomain) and an internalizing moiety portion can be made recombinantly in Pichia cells. An scFv-neprilysin may be made comprising a light chain (SEQ ID NO: 7)—linker (SEQ ID NO: 16)—a heavy chain (SEQ ID NO: 6)—linker (SEQ ID NO: 5)—neprilysin ectodomain (SEQ ID NO: 2).

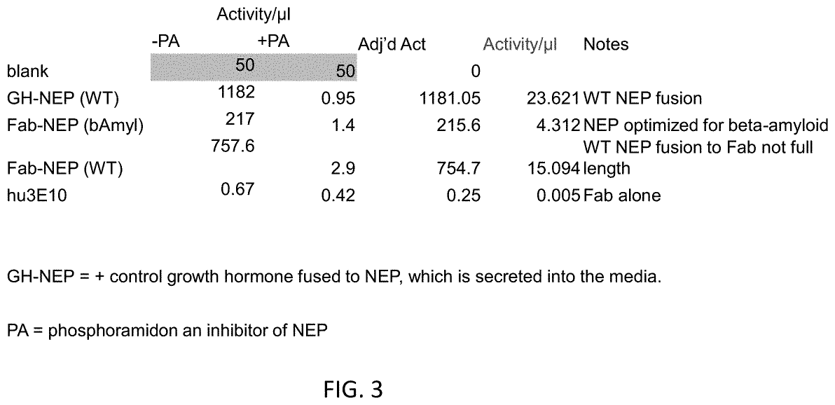

The activity of Fab-neprilysin (Fab-NEP) was examined using an activity assay with a synthetic substrate (FIG. 3). Activity was assessed for growth hormone fused to neprilysin ectodomain (GH-NEP (WT); control), Fab fused to neprilysin mutant (Fab-NEP (bAmyl)), Fab fused to wild type neprilysin ectodomain (Fab-NEP (WT)), and Fab alone (hu3E10). Concentrations were not quantitated and normalized, but activity was identified for all of the fusions. Fab-NEP (WT) appeared to demonstrate better activity than the Fab-NEP (bAmyl), but the titer of the variant Fab-NEP fusion was significantly lower than that of the wild type Fab-NEP.

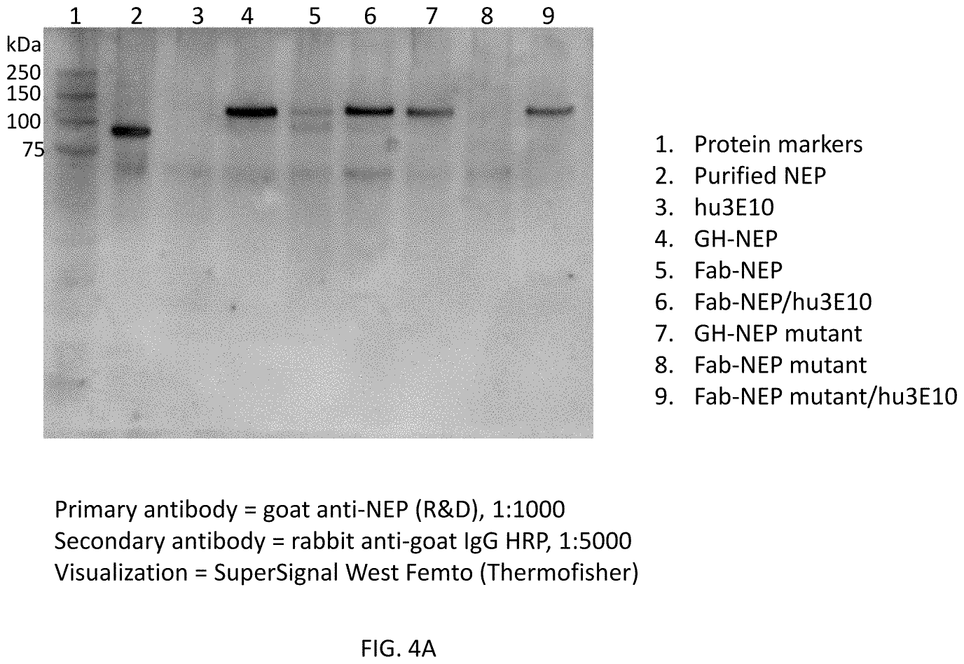

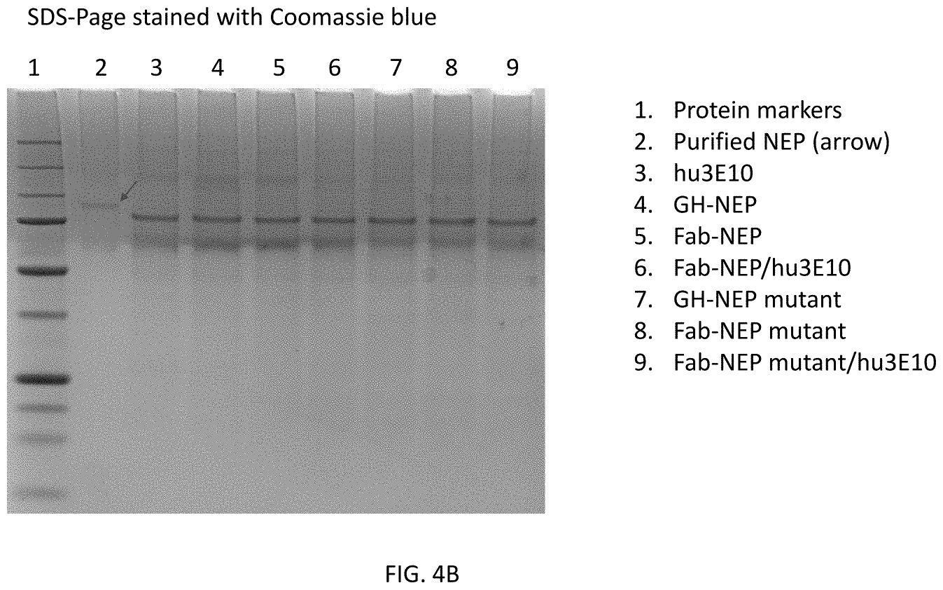

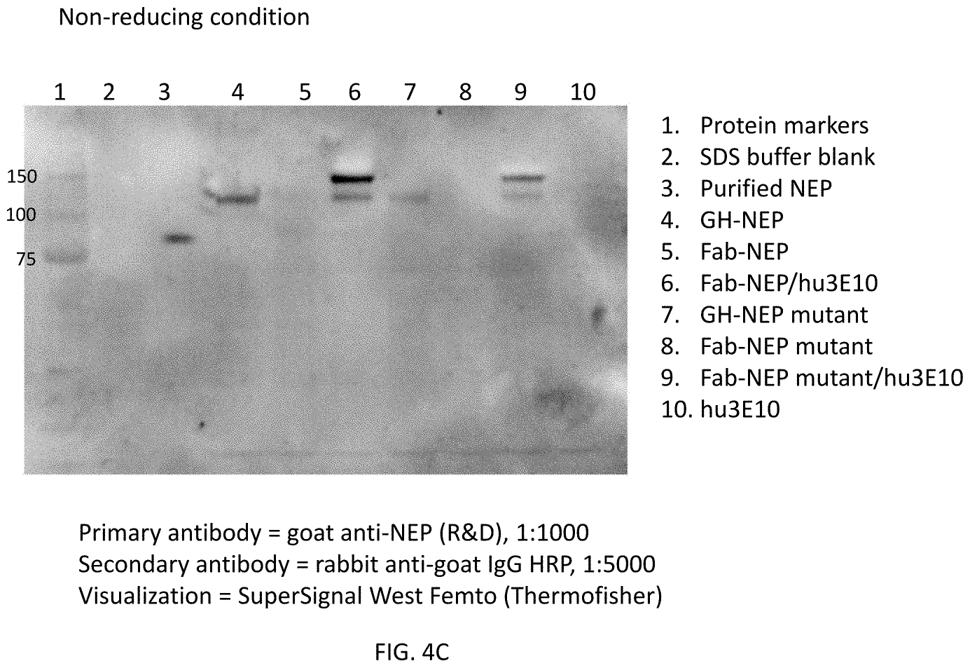

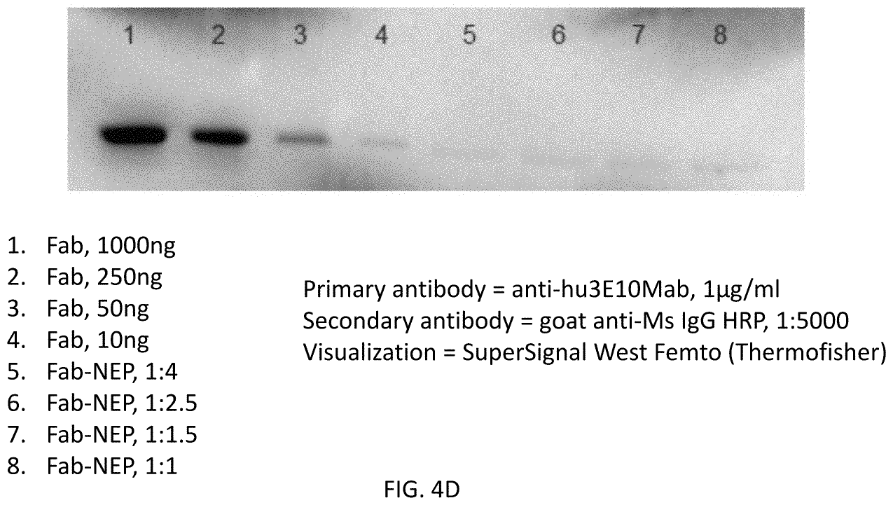

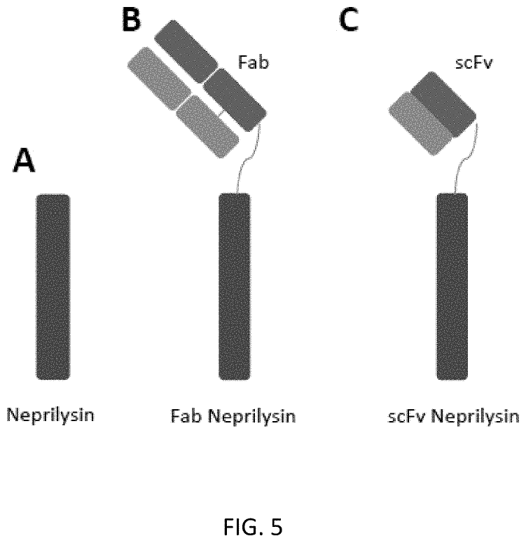

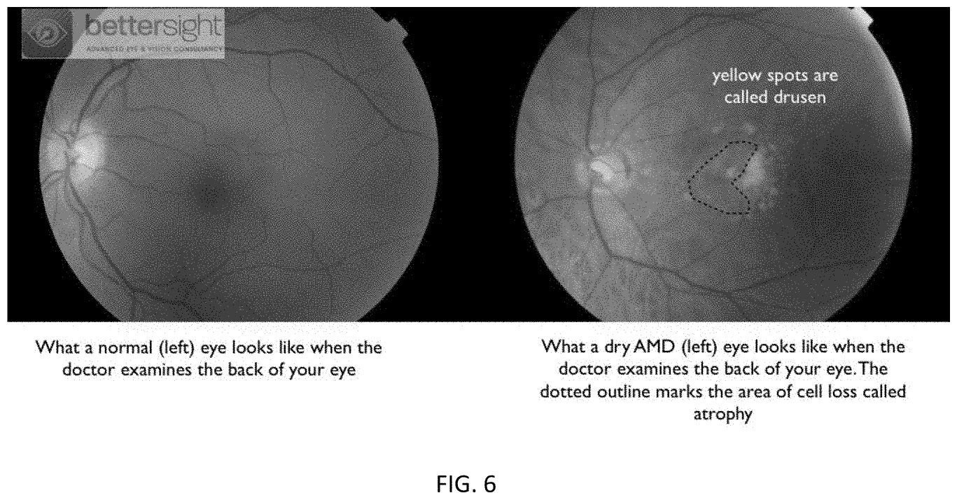

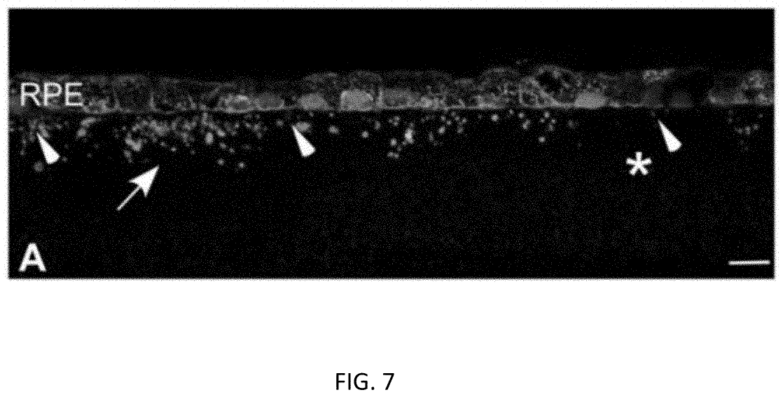

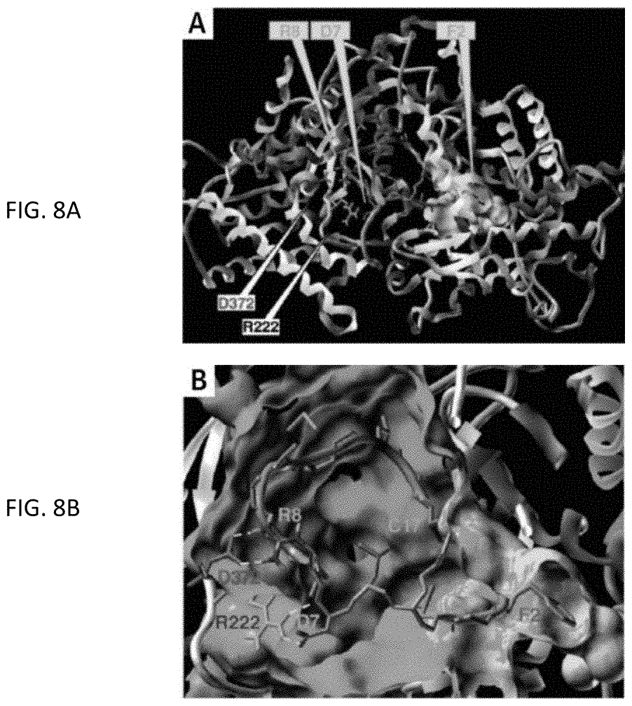

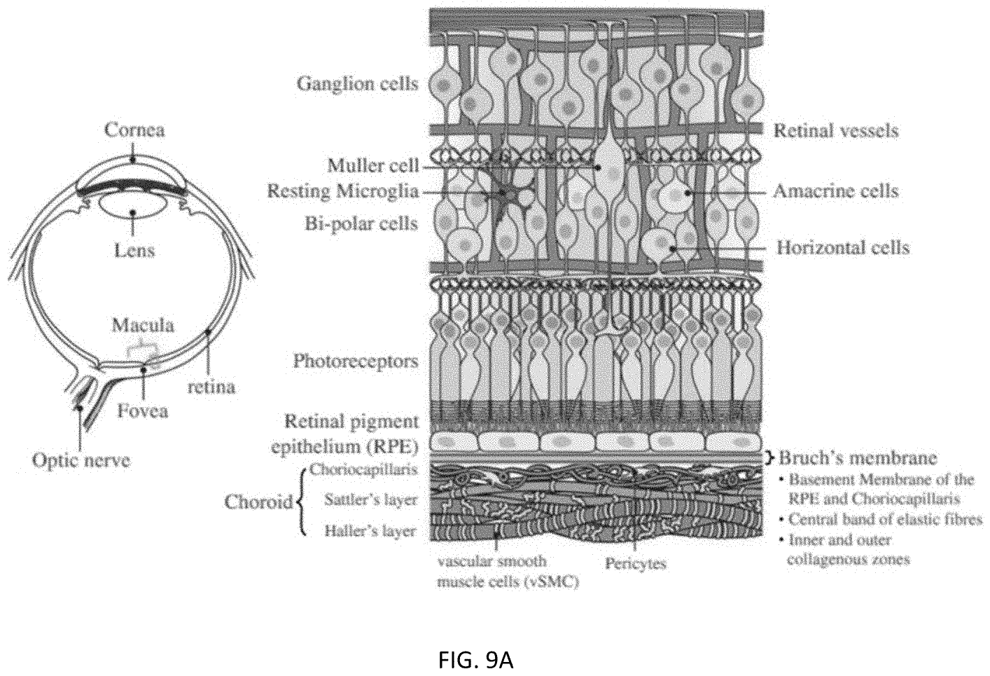

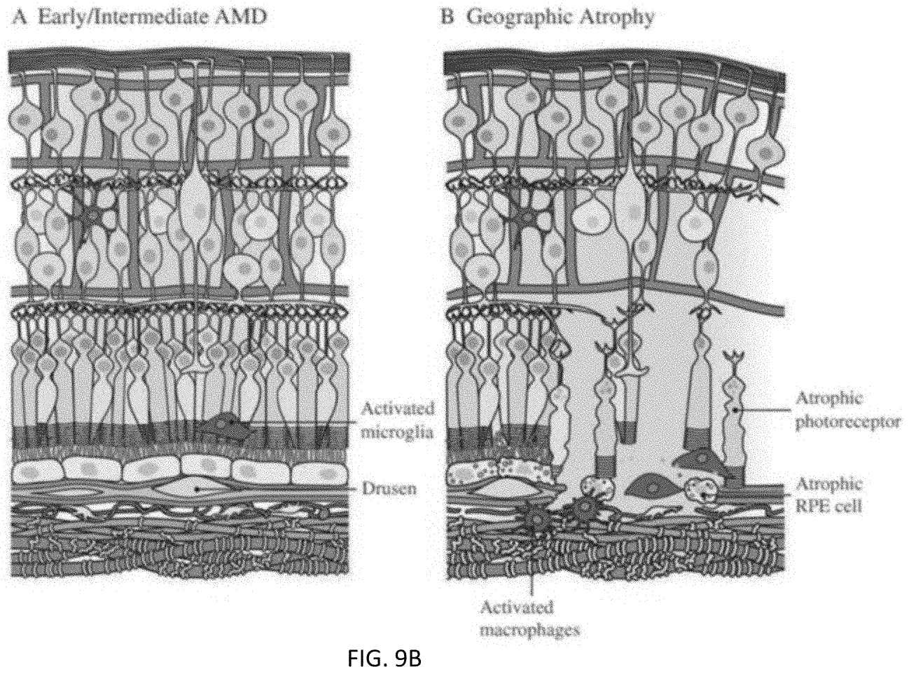

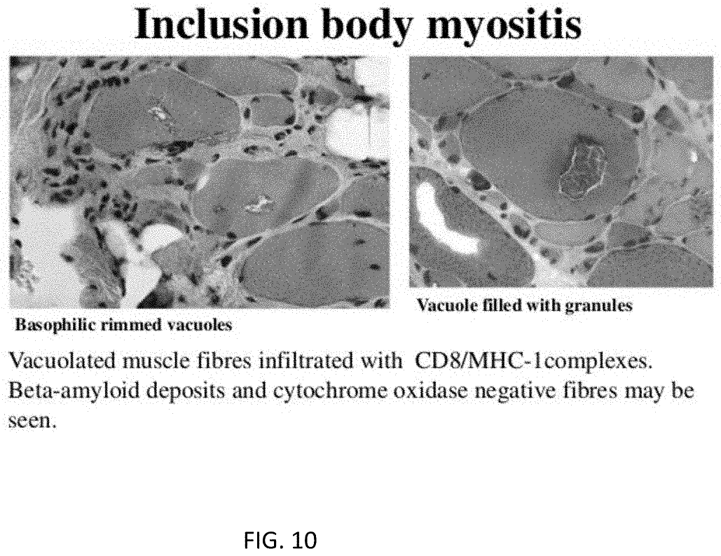

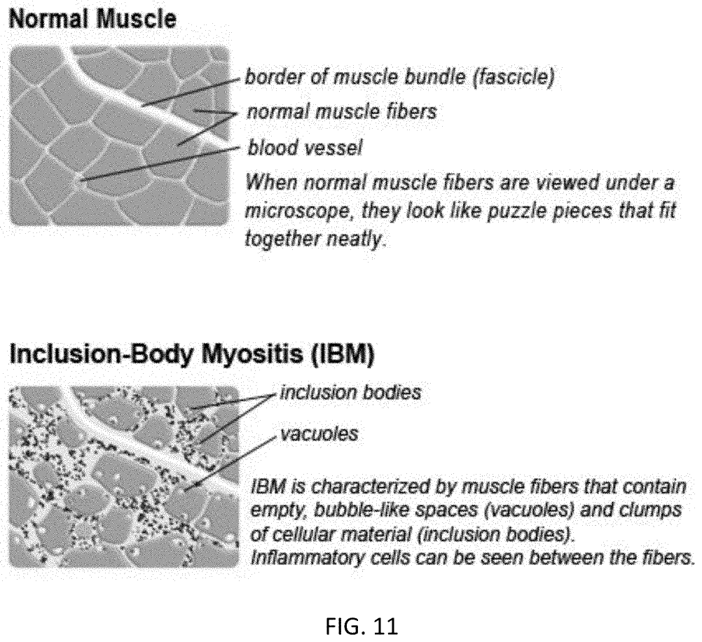

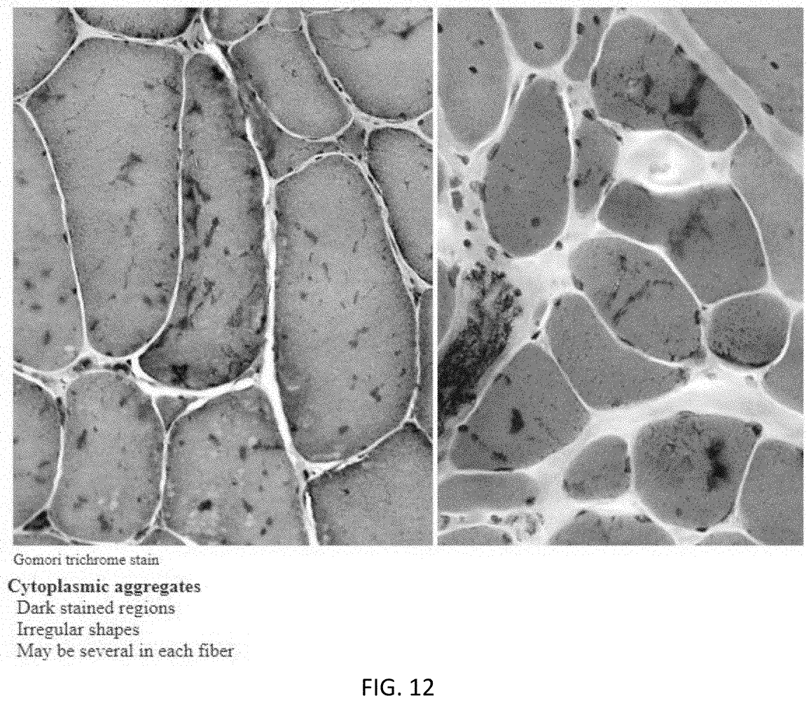

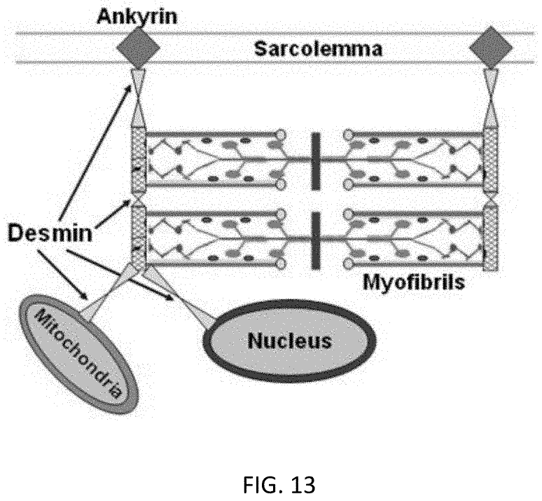

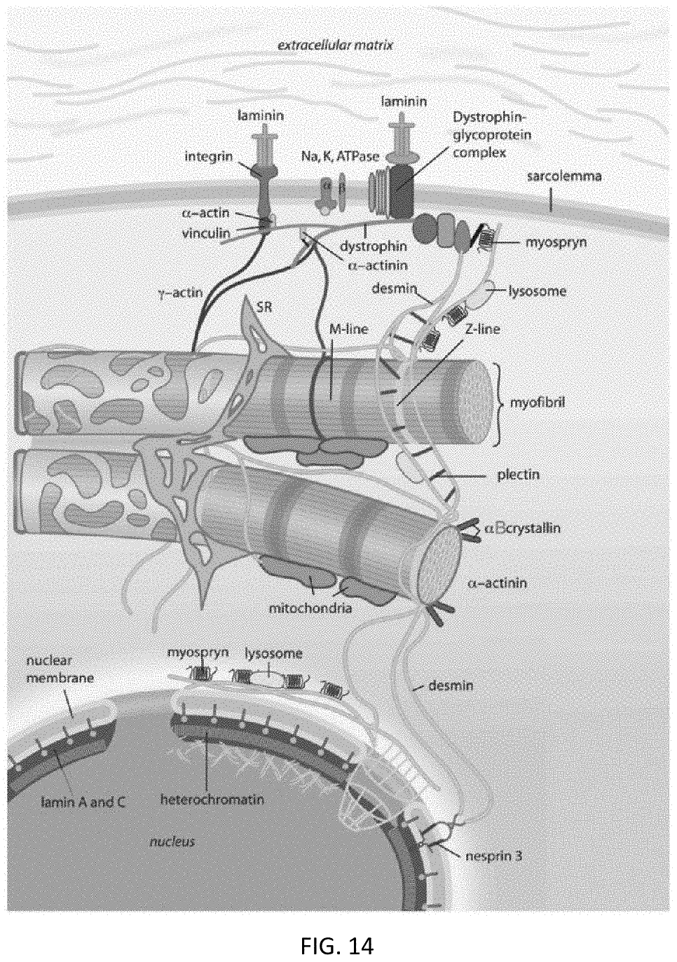

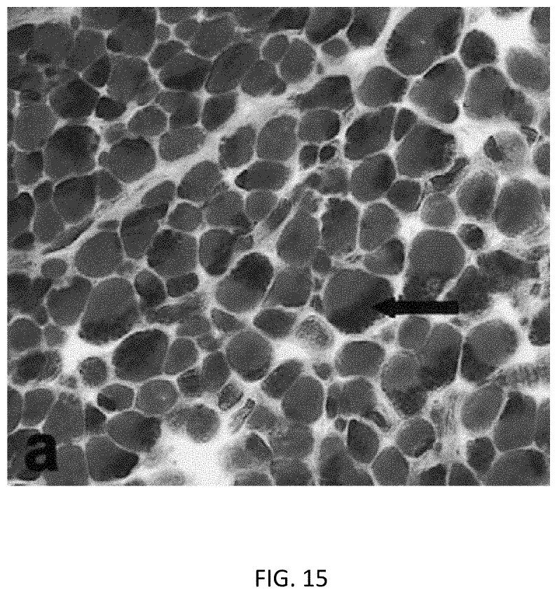

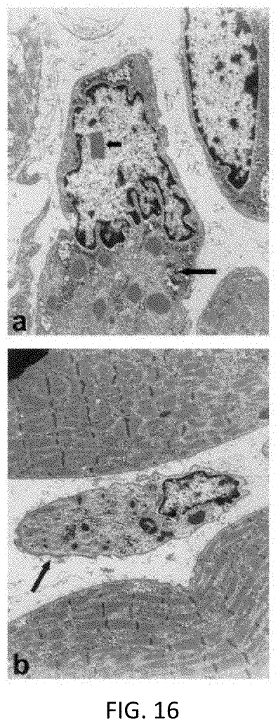

Neprilysin expression was examined in various fusions. HEK293 cells were grown in 10-cm plate with DMEM supplemented with 5% FBS. During transfection, serum free medium was used and PEI was used for transfection. PEI to DNA ratio is 3:1. Ten microgram plasmid DNA was applied. However, for the hu3E10 light chain and Fab-NEP heavy chain co-transfection, a total of 15 μg were used with 1:1 ratio. After 4 hr transfection, medium containing PEI/DNA was removed and replaced with fresh serum-free medium. After 3 days incubation at 37° C., media were collected and clarified by centrifugation. (The pH of the conditioned media dropped to 6-6.5, and 35 μl 2M Tris was added to bring back to neutral pH based on the color of phenol red). Clarified media were boiled in 4×SDS sample buffer in the presence and absence of b-ME. Forty microliter were examined on gel. The expression reports demonstrated that Fab-NEP (i.e., Fab-neprilysin ectodomain) expresses better than the Fab-NEP mutant (i.e., Fab-neprilysin mutant) (FIGS. 4A-4C). In addition, it was shown that the fusion protein (Fab-NEP) exhibited roughly equivalent expression at different transfection ratios, and that Fab-NEP secreted into the media (FIG. 4D).

### Example 2: Examination of Fab-NEP in Age-Related Macular Degeneration

The effect of Fab-neprilysin on Age-Related Macular Degeneration (AMD) can be assessed. Initially, the ability of Fab-neprilysin to breakdown muscle protein-aggregates will be assessed. One aspect for examination, is whether Fab-neprilysin get into retinal pigment epithelium (RPE) cells. ARPE-19 cells (atcc.org/Products/All/CRL-2302.aspx) may be treated with Fab-neprilysin and the cells are then pelleted, washed, lysed. The inclusion body cell lysate is then examined for neprilysin activity using anti-neprilysin antibody. Alternatively, the Fab-neprilysin is FITC labeled and flow cytometry is used to examine the effects of Fab-neprilysin. A second aspect for consideration is whether Fab-neprilysin reverses ApoE3 overexpression phenotype (increased secretion of ApoE3 that aggregates with lipids, i.e., drusen). ApoE3 may be over-expressed in ARPE-19 cells and then imaged with anti-ApoE3 antibody (Millipore) (pnas.org/content/pnas/108/45/18277.full.pdf). The cells are then treated with Fab-neprilysin and the cells may be fixed/permeabilized onto a slide, ApoE3 may be detected, and pre-versus post-treatment staining may be compared to identify any differences in appearance of protein aggregates. A third aspect for consideration is if Fab-neprilysin enters cells and/or clears protein aggregates, can it further clear lipid accumulation. Lipid accumulation can be measured in ARPE-19 cells with BODIPY dye by comparing pre-versus post-treatment.

Once Fab-neprilysin is shown to breakdown muscle protein-aggregates, production capability in bacteria and yeast will be examined. In addition, Fab-neprilysin will be characterized and formulated for TV injection. Finally, efficacy of the Fab-neprilysin will be evaluated in mouse models of AMD.

Examples of AMD animal models include:

PMID 28553324—injected Aβ into the eye and observed hallmark AMD signs due to presence of Aβ aggregates.

PMID 17888483—knocked-in hAPOE4 and fed high-fat lifestyle, generated hallmark signs of AMD. Treatment with anti-AD antibody reduced the amounts of AB in the retina and brain and ERG deficits in the retina were attenuated.

PMID 26142956-5XFAD mice that have elevated AB levels saw improvement in eye function following sNEP treatment by ERG and reduced ocular levels of AB.

### Example 3: Examination of Fab-NEP in Inclusion Body Myositis

The effect of Fab-neprilysin on Inclusion Body Myositis (IBM) can be assessed.

Initially, the ability of Fab-neprilysin to breakdown protein-aggregates using tau and 0-amyloid will be assessed. Once Fab-neprilysin is shown to breakdown protein-aggregates, production capabilities in bacteria and yeast will be examined. In addition, Fab-neprilysin will be characterized and formulated for IM injection. Finally, efficacy of the Fab-neprilysin will be evaluated in mouse models of IBM.

### Example 4: Examination of Fab-NEP in Myofibrillar Myopathy 2

The effect of Fab-neprilysin on Myofibrillar Myopathy 2 (MFM2) can be assessed. Initially, the ability of Fab-neprilysin to breakdown muscle protein-aggregates will be assessed. One aspect for examination, is whether Fab-neprilysin degrades protein aggregates in cells. CryAB R120G mutant is expressed in Sol8 cell lines (see Maloyan et al. PNAS (2007) 104(14):5995-6000) and confirm aggregate formation by Gomori trichrome staining (kit, Thermo 87020). The cells are fixed/permeabilized and then rehydrated. Fab-neprilysin is added for 1 hour at 37° C. and the cells are then stained with Gomori trichrome to identify any differences in the amount and/or size of aggregates in treated versus untreated cells. A second aspect for consideration is whether Fab-neprilysin is active in degrading aggregates in live cells. CryAB R120G mutant is expressed in Sol8 cell lines (see Maloyan et al. PNAS (2007) 104(14):5995-6000) and confirm aggregate formation by Gomori trichrome staining (kit, Thermo 87020). Fab-neprilysin is added for 24 hours at 37° C., and the cells are then stained with Gomori trichrome to identify any differences in the amount and/or size of aggregates in treated versus untreated cells. A third aspect for consideration is if Fab-neprilysin treatment rescues cell death. CryAB R120G mutant Sol8 cells are treated with Fab-neprilysin. A Western Blot is then performed for caspase cleavage. The nucleus may be observed by microscopy for fragmentation or other physical signs of cell death.

Once Fab-neprilysin is shown to breakdown muscle protein-aggregates, production capability in bacteria and yeast will be examined. In addition, Fab-neprilysin will be characterized and formulated for IM injection. Finally, efficacy of the Fab-neprilysin will be evaluated in mouse models of CryAB aggregation.

Examples of MFM2 animal models include:

010676—FVB-Tg(GFAP-CRYAB)141.6Mes/J—Mutant model. Mice express the hamster CRYAB gene under the control of the human GFAP, glial fibrillary acidic protein, promoter. Transgene expression is astrocyte specific. Mice are viable and display no phenotypic abnormalities.

027513—B6;C3-Tg(Prnp-CRYAB)Z104Dbo/J—Overexpression model. Mice express the WT human alpha B crystallin under the direction of the mouse prion protein promoter, producing approximately 6-fold overexpression of alpha beta crystallin (mouse and human) in the spinal cord, approximately 30-fold overexpression in the forebrain, and high levels of expression also detected in the cerebellum. Transgenic mice exhibit 20% more motor neurons in the spinal cord when compared to wildtype controls. Mice that are hemizygous for the transgene are viable and fertile.

### Example 5: Examination of Fab-NEP in Nemaline Myopathy

The effect of Fab-neprilysin on Nemaline Myopathy (NM) can be assessed. Initially, the ability of Fab-neprilysin to breakdown muscle protein-aggregates will be assessed. One aspect for examination, is whether Fab-neprilysin degrades thin protein filaments in cells. TPM3 M9R mutant and/or ACTA1 H40Y mutant is expressed in Sol8 cell lines (see Vandamme et al. BBA—Molecular Cell Research (2009) 1793(7):1259-1271) and confirm rod formation by Gomori trichrome staining (kit, Thermo 87020). The cells are fixed/permeabilized and then rehydrated. Fab-neprilysin is added for 1 hour at 37° C. and the cells are then stained with Gomori trichrome to identify any differences in the amount and/or size of rods in treated versus untreated cells. A second aspect for consideration is whether Fab-neprilysin is active in degrading thin protein filaments in live cells. TPM3 M9R mutant and/or ACTA1 H40Y mutant is expressed in Sol8 cell lines (see Vandamme et al. BBA—Molecular Cell Research (2009) 1793(7):1259-1271) and confirm rod formation by Gomori trichrome staining (kit, Thermo 87020). Fab-neprilysin is added for 24 hours at 37° C., and the cells are then stained with Gomori trichrome to identify any differences in the amount and/or size of rods in treated versus untreated cells. A third aspect for consideration is if Fab-neprilysin treatment rescues cell death. ACTA1 H40Y mutant Sol8 cells are treated with Fab-neprilysin. A Western Blot is then performed for caspase cleavage. The nucleus may be observed by microscopy for fragmentation or other physical signs of cell death.

Once Fab-neprilysin is shown to breakdown muscle protein-aggregates, production capability in bacteria and yeast will be examined. In addition, Fab-neprilysin will be characterized and formulated for IM injection. Finally, efficacy of the Fab-neprilysin will be evaluated in mouse models of NM.

Examples of NM animal models include:

018304—FVB/N-Tg(ACTA1-TPM3*M9R)4Hrd/MbngJ—Overexpress dominant-negative M9R mutation in TPM3. NM features: electron-dense accumulations (nemaline rods), increased slow/oxidative fiber content, and late-onset skeletal muscle weakness.

018284-129-Actaltm1Hrd/J—Endogenous α-skeletal actin sequences replaced by a mutant α-skeletal actin containing the single amino acid change (H40Y) associated with dominantly-inherited severe NM in humans.

### Example 6: Fab-NEP Production and Activity Analysis

Molecular Biology

cDNA encoding H3 Fab-linker-Neprilysin heavy chain (SEQ ID NO: 33) and cDNA encoding L2 light chain (SEQ ID NO: 35) was synthetically produced with codon optimization for mammalian cell expression and cloned into pTT5 at the indicated restriction sites (HindIII, NotI) by standard methods. Endotoxin-free plasmid DNA isolation was performed and resulted in 8.3 mg of DNA.

The DNA sequences for L2 light chain (SEQ ID NO: 35) and H3 Fab-Linker-Neprilysin heavy chain (SEQ ID NO: 33) were verified to be accurate by Sanger sequencing analysis.

The amount of DNA remaining for each following the work performed is as follows: H3 Fab-linker-Neprilysin heavy chain: 7.3 mg; L2 light chain: 6.5 mg. The theoretical pI/Mw of non-reduced secreted Fab-Neprilysin is 6.36/128481.32 Da.

Mammalian Cell Expression

Two, 1 L cultures of 293-6E cells in 2 L shake flasks were transfected with 1 mg of plasmid DNA/L culture (1:1 ratio HC:LC) using PolyPlus linear PEIpro at a 1:1 (w/v) DNA:PEI ratio. Culture parameters were monitored using a ViCell XR for density, viability, and average diameter measurements (average of 50 determinations). Culture media was F17 supplemented with 0.1% Pluronic F-68, 4 mM GlutaMAX, 25 μg/mL G418. Initial density of the 293-6E cells was 2.1×10⁶ cells/mL. The 293-6E cells were maintained at a density between 0.4-4.0×10⁶ cells/mL in shake flasks. The flasks were incubated at 37° C. in a humidified 5% CO₂ environment with shaking at 135 rpm.

TABLE 1
Culture parameters

| | Volume | Time | Density (×10⁶ | Viability |
|---|---|---|---|---|
| Sample | (L) | (days) | viable cells/mL) | (%) |
| Fab-NEP | 2 | 0 | 2.1 | 99 |
| | | 1 | 3.3 | 98 |
| | | 2 | 3.6 | 98 |
| | | 5 | 3.8 | 79 |
| | | 6 | 3.1 | 62 |

The culture was harvested 6 days post transfection via centrifugation at 4° C. for 5 minutes at 1000×g. The conditioned culture supernatant (CCS) was clarified by centrifugation at 4° C. for 30 minutes at 9300×g. Expression was then verified.

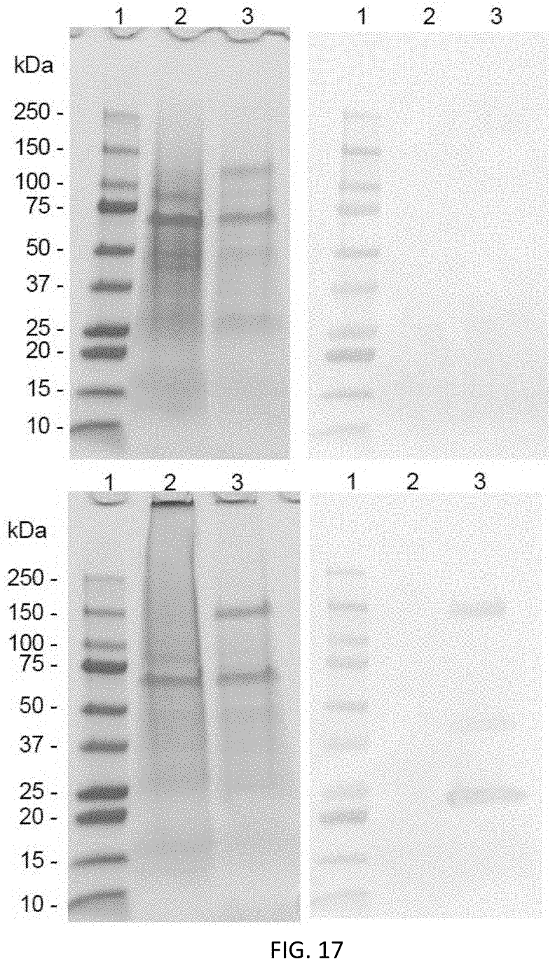

Forty microliters of CCS was evaluated via reducing (FIG. 17, top panels) and non-reducing (FIG. 17, bottom panels) SDS-PAGE on 4-20% Tris-glycine TGX gels (Bio-Rad), stained with Instant Blue gel stain (FIG. 17, left panels) or western blotted to PVDF (FIG. 17, right panels). The western blots were incubated with rabbit anti-Neprilysin 1° antibody (Millipore Sigma #AB5458) at a 1:1000 dilution and goat anti-Rabbit IgG-AP 2° antibody (Bio-Rad #170-6518) at a 1:1000 dilution. Immunoreactivity was detected using an alkaline phosphatase conjugate substrate kit (Bio-Rad).

Purification

The target protein in the conditioned culture supernatant was batch bound to 10 mL of IgG-CH1 XL resin at 4° C. overnight with stirring. The resin was collected in a 2.5 cm diameter Econo-column. Resin was washed with 15 column volumes (CV) of Buffer A (1×PBS (2.7 mM KCl, 1.7 mM KH₂PO₄, 136 mM NaCl, 10.1 mM Na₂HPO₄), pH 7.4 (23° C.)). Elution was 50 mM sodium acetate, pH 4.5 (23° C.) was attempted, but the target protein remained associated with the resin. The protein of interest was eluted in 4 CV of Buffer B (50 mM sodium acetate, pH 4.0 (23° C.)) and immediately diluted into 1/10 volume of Buffer D (1 M Tris, pH 8.0 (23° C.)) to neutralize the solution. The elution procedure was repeated with 2 CV of Buffer C (30 mM sodium acetate, pH 3.6 (23° C.)) immediately diluted into 1/10 volume of Buffer D. Fractions (5 mL) were collected and analyzed by A280 prior to separately pooling pH 4.0 elution fractions 4-6 and pH 3.6 elution fractions 2-4 (see Table 2).

TABLE 2
Affinity Chromatography

| | Pooled | Pool Volume |
|---|---|---|
| Elution Pool | Fractions | (mL) |
| pH 4.0 | 4-6 | 15 |
| pH 3.6 | 2-4 | 15 |

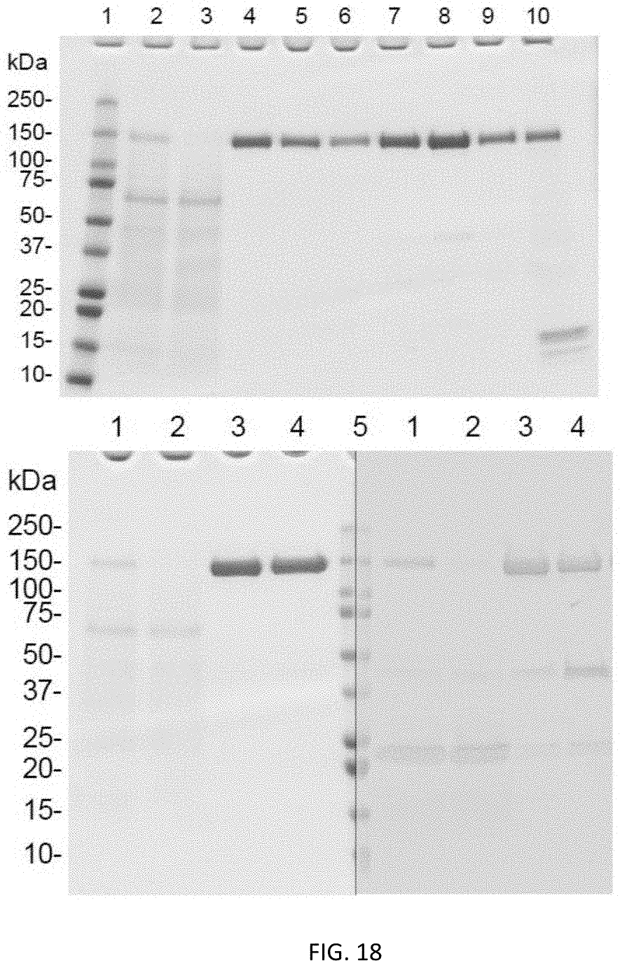

Select fractions were analyzed by SDS-PAGE (FIG. 18). 4-20% Tris-glycine TGX gels were stained with Instant Blue gel stain (FIG. 18, top panel and bottom left panel) or western blotted to PVDF (bottom right panel). The western blot was incubated with rabbit anti-Neprilysin 1° antibody (Millipore Sigma #AB5458) at a 1:1000 dilution and goat anti-Rabbit IgG-AP 2° antibody (Bio-Rad #170-6518) at a 1:1000 dilution. Immunoreactivity was detected using an alkaline phosphatase conjugate substrate kit (Bio-Rad).

The pH 4.0 and pH 3.6 elution pools were dialyzed against 3×3 L of 20 mM HEPES, 140 mM NaCl, pH 7.5 (23° C.). The dialyzed pools were concentrated (Table 3) using VivaSpin20 (10K molecular weight cutoff, PES membrane) centrifugal devices prior to final analysis and storage.

TABLE 3
Dialysis and Concentration

| | Final concentration |
|---|---|
| Fab-Neprilysin pool | (mg/mL) |
| pH 4.0 elution | 3.2 |
| pH 3.6 elution | 5.4 |

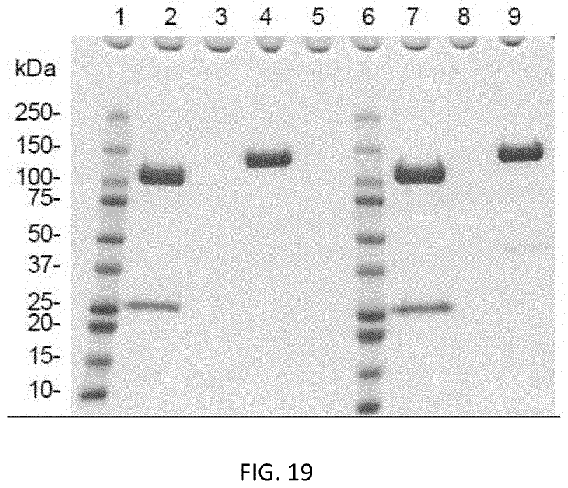

Final samples were analyzed with 4-20% Tris-glycine TGX gel, reducing and non-reducing SDS-PAGE, stained with Instant Blue stain (FIG. 19).

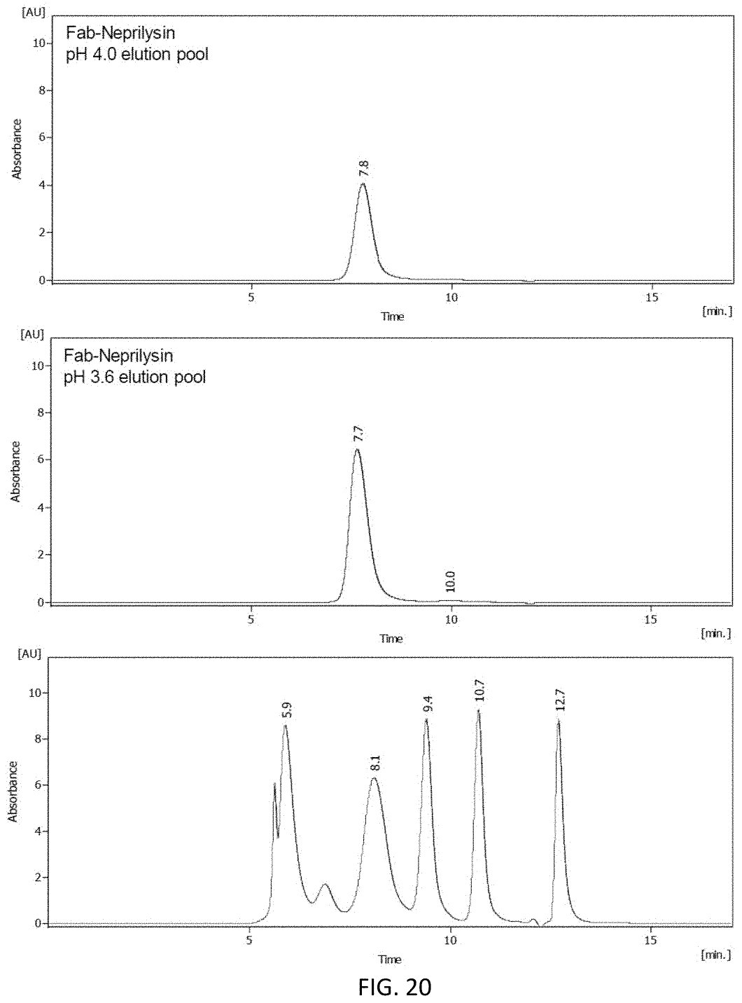

In additional, analytical size exclusion chromatography was performed using an Agilent HP1100 unit #2 to provide a freeze/thaw analysis. Samples of the final protein pools were injected on the equilibrated column (7.8×300 mm) (10 μL injection volume) and the protein was eluted isocratically over 1.1 CV. Samples that had been frozen at −80° C. and thawed at room temperature were analyzed using the same method. Resin was a TSKgel G3000SW×1 equilibrated in 50 mM NaH₂PO₄, 0.2 M L-arginine, pH 6.8 (23° C.). Buffer was 50 mM NaH2PO4, 0.2 M L-arginine, pH 6.8 (23 C). Flow rate was 1 mL/min. Detection wavelength was 230 nm (standards and samples). Analytical SEC chromatograms were obtained with final samples in blue (FIG. 20, upper and middle panels), final samples after freeze/thaw in red (FIG. 20, upper and middle panels; nearly indistinguishable from final sample) and molecular weight standard in blue (FIG. 20, lower panel). Molecular weight standards are provided in Table 4 and monodispersity data is provided in Table 5.

TABLE 4
Molecular Weight Standards
MW Standards

| | Peak Retention | |
|---|---|---|
| Peak # | Time (min) | MW (Da) |
| 1 | 5.9 | 670,000 |
| 2 | 8.1 | 158,000 |
| 3 | 9.4 | 44,000 |
| 4 | 10.7 | 17,000 |
| 5 | 12.7 | 1,350 |

TABLE 5
Monodispersity Data

| | % non-monodisperse | % non-monodisperse |
|---|---|---|
| Protein Sample | pre-freeze/thaw | post-freeze/thaw |
| Fab-Neprilysin | <0.1% | <0.1% |
| pH 4.0 elution pool | | |
| Fab-Neprilysin | 1.4% | 1.4% |
| pH 3.6 elution pool | | |

TABLE 6
Fab-Neprilysin pH 4.0 Elution Summary Table

| Fab-Neprilysin | Volume | Concentration | Total protein |
|---|---|---|---|
| pH 4.0 elution | (mL) | (mg/mL)* | (mg) |
| IgG-CH1 XL pool | 15.00 | 0.3 | 4.5 |
| Dialyzed pool | 14.50 | 0.3 | 4.4 |
| Concentrated final pool | 1.10 | 3.2 | 3.5 |
| Delivered pool | 1.04 | 3.2 | 3.2 |

*Extinction coefficient A280 of 1-1.5 mg/mL

TABLE 7
Fab-Neprilysin pH 3.6 Elution Summary Table

| Fab-Neprilysin | Volume | Concentration | Total protein |
|---|---|---|---|
| pH 4.0 elution | (mL) | (mg/mL)* | (mg) |
| IgG-CH1 XL pool | 15.00 | 0.5 | 7.5 |
| Dialyzed pool | 14.70 | 0.5 | 7.4 |
| Concentrated final pool | 1.20 | 5.4 | 6.5 |
| Delivered pool | 1.15 | 5.4 | 6.2 |

*Extinction coefficient A280 of 1-1.5 mg/mL
Note:
~3.4 mg of protein remained associated with the IgG-CH1 XL resin, as evidenced by stripping with 100 mM Glycine, pH 2.6 (23° C.).

TABLE 8
Aliquot Information

| Delivered protein | Aliquots |
|---|---|
| Fab-Neprilysin | 3 × 0.313 mL |
| pH 4.0 elution pool | 1 × 0.100 mL |
| Fab-Neprilysin | 6 × 0.185 |
| pH 3.6 elution pool | 1 × 0.0.40 |

Note:
storage temperature was −80° C. (flash frozen in liquid nitrogen); final destination buffer was 20 mM HEPES, 140 mM NaCl, pH 7.5 (prepared at 23° C.).

Fab-NEP Activity Assay

Activity assay data was obtained for three resulting Fab-NEP elution pools produced, as described above.

TABLE 9
GAAF Assay

| | conc | | assay vol. | dilution | | | |
|---|---|---|---|---|---|---|---|
| Sample | (mg/mL) | rate | (μL) | fold | units/μL | units/μg | ratio |
| yNEP | 3.6 | 890 | 10 | 400 | 35600 | 9889 | 21672 |
| FabNEP 2.5 | 0.125 | 445.1 | 5 | 10 | 890 | 7122 | 14205 |
| FabNEP 3.6 | 5.4 | 1484.05 | 2 | 100 | 74203 | 13741 | 22604 |
| FabNEP 4.0 | 3.2 | 1427.55 | 3 | 100 | 47585 | 14870 | 22660 |

*rate = flu/min

TABLE 10
Abeta HPLC

| | conc | | assay vol. | dilution | | | |
|---|---|---|---|---|---|---|---|
| Sample | (mg/mL) | rate | (μL) | fold | units/μL | unils/μg | per hr. |
| yNEP | 3.6 | 0.0137 | 0.5 | 1 | 0.0274 | 0.008 | 0.46 |
| FabNEP 2.5 | 0.125 | 0.0104 | 10 | 1 | 0.001 | 0.008 | 0.5 |
| FabNEP 3.6 | 5.4 | 0.0137 | 2.5 | 10 | 0.0547 | 0.01 | 0.61 |
| FabNEP 4.0 | 3.2 | 0.0175 | 5 | 10 | 0.035 | 0.011 | 0.66 |

*rate = million sq unt/min

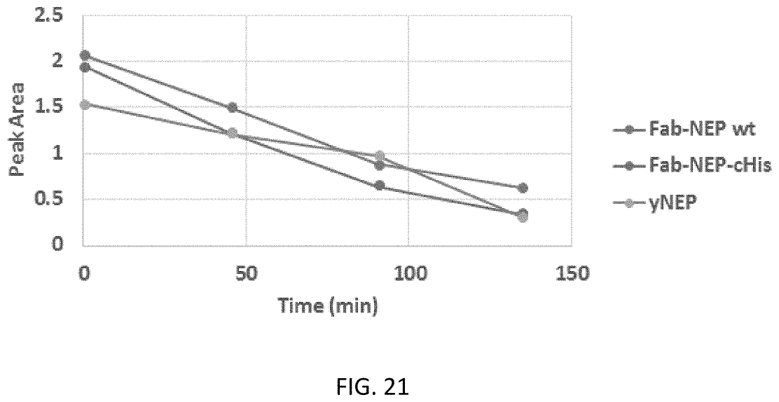

Neprilysin degradation of β-amyloid was quantified by monitoring the disappearance of the β-amyloid peak by HPLC measured by absorbance at 214 nm. Fab-NEP, Fab-NEP-cHis, and yNEP were individually mixed with 10 μM β-amyloid and incubated for 0, 45, 90, or 135 minutes at 37° C. The samples were then injected onto a C4 RP-HPLC column and eluted with an increasing gradient of acetonitrile. Peak area was determined using Waters Empower 2 software (FIG. 21).

Fab-NEP Cell Uptake Assay

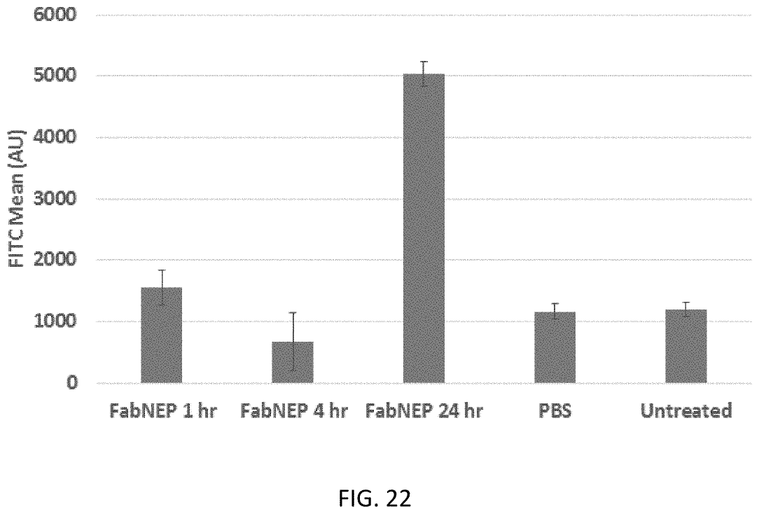

T47D cells were plated at 150K cells/well in 96 well plates. Fab-NEP, conjugated to FITC at 0.1% v/v, was added to the media and incubated for the noted time period. Cells were fixed in 1% PFA prior to recording 10,000 events on LSR II (BD Biosciences). Events were gated according to size, live/dead stain intensity, and FITC signal (FIG. 22).

Conclusions

Fab-NEP can be expressed and purified from HEK cell media with a high degree of purity. The Fab portion does not inhibit degradation of β-amyloid (1-42). Fab-NEP is internalized by T47D cells after 24 hour incubation.

### Example 7: Examination of Fab-NEP Effect on β-Amyloid

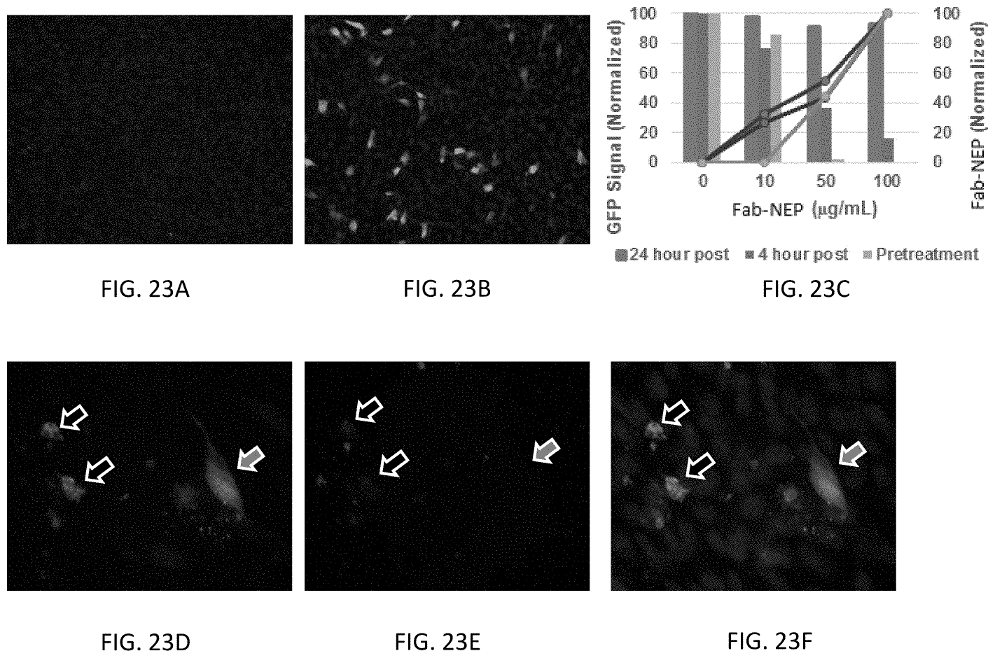

C2C12 cells were either pre-treated with 100 μg/mL Fab-NEP for 24 hours, or were not pre-treated with Fab-NEP. The cells were then transfected with 500 ng GFP reporter plasmid (β-amyloid (1-42) fused to a GFP reporter). After 24 hours of incubation, the cells were washed and fixed in PFA. Images were taken at 10× (FIGS. 23A-23B) and 40× (FIGS. 23D-23F) to visual the accumulation of β-amyloid.

Fab-NEP was shown to prevent the accumulation of β-amyloid(1-42) in C2C12 cells in a dose-dependent manner, but does not appear to clear existing agreegates. Neprilysin has been shown to degrade single-chain polypeptides (e.g., monomers or small oligomers), but it is generally understood that it may not be able to penetrate/degrade larger aggregates that have few, if any, accessible solvent-exposed single chains for neprilysin to attach to.

### Exemplary Sequences

SEQ ID NO: 1
Neprilysin amino acid sequence (GenBank Accession No:
NP_001341571)
MGKSESQMDITDINTPKPKKKQRWTPLEISLSVLVLLLTIIAVTMIALYATYDDGIC
KSSDCIKSAARLIQNMDATTEPCTDFFKYACGGWLKRNVIPETSSRYGNFDILRDEL
EVVLKDVLQEPKTEDIVAVQKAKALYRSCINESAIDSRGGEPLLKLLPDIYGWPVA
TENWEQKYGASWTAEKAIAQLNSKYGKKVLINLFVGTDDKNSVNHVIHIDQPRLG
LPSRDYYECTGIYKEACTAYVDFMISVARLIRQEERLPIDENQLALEMNKVMELEK
EIANATAKPEDRNDPMLLYNKMTLAQIQNNFSLEINGKPFSWLNFTNEIMSTVNISI
TNEEDVVVYAPEYLTKLKPILTKYSARDLQNLMSWRFIMDLVSSLSRTYKESRNAF
RKALYGTTSETATWRRCANYVNGNMENAVGRLYVEAAFAGESKHVVEDLIAQIR
EVFIQTLDDLTWMDAETKKRAEEKALAIKERIGYPDDIVSNDNKLNNEYLELNYKE
DEYFENIIQNLKFSQSKQLKKLREKVDKDEWISGAAVVNAFYSSGRNQIVFPAGILQ
PPFFSAQQSNSLNYGGIGMVIGHEITHGFDDNGRNFNKDGDLVDWWTQQSASNFK
EQSQCMVYQYGNFSWDLAGGQHLNGINTLGENIADNGGLGQAYRAYQNYIKKNG
EEKLLPGLDLNHKQLFFLNFAQVWCGTYRPEYAVNSIKTDVHSPGNFRIIGTLQNS
AEFSEAFHCRKNSYMNPEKKCRVW
SEQ ID NO: 2
Neprilysin polypeptide amino acid sequence comprising Neprilysin
ectodomain (residues 52-750)
YDDGICKSSDCIKSAARLIQNMDATTEPCTDFFKYACGGWLKRNVIPETSSRYGNF
DILRDELEVVLKDVLQEPKTEDIVAVQKAKALYRSCINESAIDSRGGEPLLKLLPDI
YGWPVATENWEQKYGASWTAEKAIAQLNSKYGKKVLINLFVGTDDKNSVNHVIH
IDQPRLGLPSRDYYECTGIYKEACTAYVDFMISVARLIRQEERLPIDENQLALEMNK
VMELEKEIANATAKPEDRNDPMLLYNKMTLAQIQNNFSLEINGKPFSWLNFTNEIM
STVNISITNEEDVVVYAPEYLTKLKPILTKYSARDLQNLMSWRFIMDLVSSLSRTYK
ESRNAFRKALYGTTSETATWRRCANYVNGNMENAVGRLYVEAAFAGESKHVVED
LIAQIREVFIQTLDDLTWMDAETKKRAEEKALAIKERIGYPDDIVSNDNKLNNEYLE
LNYKEDEYFENIIQNLKFSQSKQLKKLREKVDKDEWISGAAVVNAFYSSGRNQIVF
PAGILQPPFFSAQQSNSLNYGGIGMVIGHEITHGFDDNGRNFNKDGDLVDWWTQQ
SASNFKEQSQCMVYQYGNFSWDLAGGQHLNGINTLGENIADNGGLGQAYRAYQN
YIKKNGEEKLLPGLDLNHKQLFFLNFAQVWCGTYRPEYAVNSIKTDVHSPGNFRII
GTLQNSAEFSEAFHCRKNSYMNPEKKCRVW
SEQ ID NO: 3
humanized variable heavy chain amino acid sequence
EVQLQESGGGVVQPGGSLRLSCAASGFTFSNYGMHWIRQAPGKGLEWVSYISSGSS
TIYYADSVKGRFTISRDNSKNTLYLQMNSLRSEDTAVYYCARRGLLLDYWGQGTL
VTVSS
SEQ ID NO: 4
humanized variable light chain amino acid sequence
DIQMTQSPSSLSASVGDRVTISCRASKSVSTSSYSYMHWYQQKPEKAPKLLIKYAS
YLQSGVPSRFSGSGSGTDFTLTISSLQPEDVATYYCQHSREFPWTFGAGTKLELK
SEQ ID NO: 5
glycine-serine linker amino acid sequence
GGSGGGSGGGSGG
SEQ ID NO: 6
humanized heavy chain Fd amino acid sequence (including human IgG1
CH1 and truncated hinge constant regions)
EVQLQESGGGVVQPGGSLRLSCAASGFTFSNYGMHWIRQAPGKGLEWVSYISSGSS
TIYYADSVKGRFTISRDNSKNTLYLQMNSLRSEDTAVYYCARRGLLLDYWGQGTL
VTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKKVEPKSCDKTHT
SEQ ID NO: 7
humanized light chain amino acid sequence (including human kappa
light chain region)
DIQMTQSPSSLSASVGDRVTISCRASKSVSTSSYSYMHWYQQKPEKAPKLLIKYAS
YLQSGVPSRFSGSGSGTDFTLTISSLQPEDVATYYCQHSREFPWTFGAGTKLELKRT
VAAPSVFIFPPSDEQLKSGTASVVCLLNNFYPREAKVQWKVDNALQSGNSQESVTE
QDSKDSTYSLSSTLTLSKADYEKHKVYACEVTHQGLSSPVTKSFNRGEC
SEQ ID NO: 8
heavy chain IgG1 CH1 and truncated hinge constant domain
ASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDKTHT
SEQ ID NO: 9
human kappa constant domain of Km3 allotype
RTVAAPSVFIFPPSDEQLKSGTASVVCLLNNFYPREAKVQWKVDNALQSGNSQESV
TEQDSKDSTYSLSSTLTLSKADYEKHKVYACEVTHQGLSSPVTKSFNRGEC
SEQ ID NO: 10
heavy chain variable (VH) domain CDR1 of exemplary 3E10 molecule,
in accordance with CDRs as defined by the IMGT system
GFTFSNYG
SEQ ID NO: 11
heavy chain variable (VH) domain CDR2 of exemplary 3E10 molecule,
in accordance with CDRs as defined by the IMGT system
ISSGSSTI
SEQ ID NO: 12
heavy chain variable (VH) domain CDR3 of exemplary 3E10 molecule,
in accordance with CDRs as defined by the IMGT system
ARRGLLLDY
SEQ ID NO: 13
light chain variable (VL) domain CDR1 of exemplary 3E10 molecule, in
accordance with CDRs as defined by the IMGT system
KSVSTSSYSY
SEQ ID NO: 14
light chain variable (VL) domain CDR2 of exemplary 3E10 molecule, in
accordance with CDRs as defined by the IMGT system
YAS
SEQ ID NO: 15
light chain variable (VL) domain CDR3 of exemplary 3E10 molecule, in
accordance with CDRs as defined by the IMGT system
QHSREFPWT
SEQ ID NO: 16
G53 linker
GGGGSGGGGSGGGGS
SEQ ID NO: 17
amino acid sequence of murine 3E10 heavy chain variable domain (VH)
EVQLVESGGGLVKPGGSRKLSCAASGFTFSNYGMHWVRQAPEKGLEWVAYISSGS
STIYYADTVKGRFTISRDNAKNTLFLQMTSLRSEDTAMYYCARRGLLLDYWGQGT
TLTVSS
SEQ ID NO: 18
amino acid sequence of murine 3E10 light chain variable domain (VL)
DIVLTQSPASLAVSLGQRATISCRASKSVSTSSYSYMHWYQQKPGQPPKLLIKYASY
LESGVPARFSGSGSGTDFHLNIHPVEEEDAATYYCQHSREFPWTFGGGTKLELK
SEQ ID NO: 19
heavy chain variable domain CDR1 of 3E10 VH (as that VH is defined
with reference to SEQ ID NO: 17), in accordance with Kabat system
NYGMH
SEQ ID NO: 20
heavy chain variable domain CDR2 of 3E10 VH (as that VH is defined
with reference to SEQ ID NO: 17), in accordance with Kabat system
YISSGSSTIYYADTVKG
SEQ ID NO: 21
heavy chain variable domain CDR3 of 3E10 VH (as that VH is defined
with reference to SEQ ID NO: 17), in accordance with Kabat system
RGLLLDY
SEQ ID NO: 22
light chain variable domain CDR1 of 3E10 VL (as that VL is defined
with reference to SEQ ID NO: 18), in accordance with Kabat system
RASKSVSTSSYSYMH
SEQ ID NO: 23
light chain variable domain CDR2 of 3E10 VL (as that VL is defined
with reference to SEQ ID NO: 18), in accordance with Kabat system
YASYLES
SEQ ID NO: 24
light chain variable domain CDR3 of 3E10 VL (as that VL is defined
with reference to SEQ ID NO: 18), in accordance with Kabat system
QHSREFPWT
SEQ ID NO: 25
heavy chain variable domain CDR2 of certain antibodies of the
disclosure, in accordance with CDRs as defined by Kabat
YISSGSSTIYYADSVKG
SEQ ID NO: 26
light chain variable domain CDR1 of certain antibodies of the
disclosure, in accordance with CDRs as defined by Kabat
RASKSVSTSSYSYLA
SEQ ID NO: 27
light chain variable domain CDR2 of certain antibodies of the
disclosure, in accordance with CDRs as defined by Kabat
YASYLQS
SEQ ID NO: 28
reference humanized Fv3E10
DIQMTQSPSSLSASVGDRVTISCRASKSVSTSSYSYMHWYQQKPEKAPKLLIKYAS
YLQSGVPSRFSGSGSGTDFTLTISSLQPEDVATYYCQHSREFPWTFGAGTKLELKGG
GGSGGGGSGGGGSEVQLQESGGGVVQPGGSLRLSCAASGFTFSNYGMHWIRQAPG
KGLEWVSYISSGSSTIYYADSVKGRFTISRDNSKNTLYLQMNSLRSEDTAVYYCAR
RGLLLDYWGQGTLVTVSS
SEQ ID NO: 29
heavy chain + neprilysin ectodomain polypeptide amino acid sequence
EVQLQESGGGVVQPGGSLRLSCAASGFTFSNYGMHWIRQAPGKGLEWVSYISSGSS
TIYYADSVKGRFTISRDNSKNTLYLQMNSLRSEDTAVYYCARRGLLLDYWGQGTL
VTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKKVEPKSCDKTHT
GGSGGGSGGGSGGYDDGICKSSDCIKSAARLIQNMDATTEPCTDFFKYACGGWLK
RNVIPETSSRYGNFDILRDELEVVLKDVLQEPKTEDIVAVQKAKALYRSCINESAIDS
RGGEPLLKLLPDIYGWPVATENWEQKYGASWTAEKAIAQLNSKYGKKVLINLFVG
TDDKNSVNHVIHIDQPRLGLPSRDYYECTGIYKEACTAYVDFMISVARLIRQEERLP
IDENQLALEMNKVMELEKEIANATAKPEDRNDPMLLYNKMTLAQIQNNFSLEING
KPFSWLNFTNEIMSTVNISITNEEDVVVYAPEYLTKLKPILTKYSARDLQNLMSWRF
IMDLVSSLSRTYKESRNAFRKALYGTTSETATWRRCANYVNGNMENAVGRLYVE
AAFAGESKHVVEDLIAQIREVFIQTLDDLTWMDAETKKRAEEKALAIKERIGYPDDI
VSNDNKLNNEYLELNYKEDEYFENIIQNLKFSQSKQLKKLREKVDKDEWISGAAV
VNAFYSSGRNQIVFPAGILQPPFFSAQQSNSLNYGGIGMVIGHEITHGFDDNGRNFN
KDGDLVDWWTQQSASNFKEQSQCMVYQYGNFSWDLAGGQHLNGINTLGENIAD
NGGLGQAYRAYQNYIKKNGEEKLLPGLDLNHKQLFFLNFAQVWCGTYRPEYAVN
SIKTDVHSPGNFRIIGTLQNSAEFSEAFHCRKNSYMNPEKKCRVW
SEQ ID NO: 30
scFv-neprilysin
DIQMTQSPSSLSASVGDRVTISCRASKSVSTSSYSYMHWYQQKPEKAPKLLIKYAS
YLQSGVPSRFSGSGSGTDFTLTISSLQPEDVATYYCQHSREFPWTFGAGTKLELKGG
GGSGGGGSGGGGSEVQLQESGGGVVQPGGSLRLSCAASGFTFSNYGMHWIRQAPG
KGLEWVSYISSGSSTIYYADSVKGRFTISRDNSKNTLYLQMNSLRSEDTAVYYCAR
RGLLLDYWGQGTLVTVSSGGSGGGSGGGSGGYDDGICKSSDCIKSAARLIQNMDA
TTEPCTDFFKYACGGWLKRNVIPETSSRYGNFDILRDELEVVLKDVLQEPKTEDIVA
VQKAKALYRSCINESAIDSRGGEPLLKLLPDIYGWPVATENWEQKYGASWTAEKAI
AQLNSKYGKKVLINLFVGTDDKNSVNHVIHIDQPRLGLPSRDYYECTGIYKEACTA
YVDFMISVARLIRQEERLPIDENQLALEMNKVMELEKEIANATAKPEDRNDPMLLY
NKMTLAQIQNNFSLEINGKPFSWLNFTNEIMSTVNISITNEEDVVVYAPEYLTKLKPI
LTKYSARDLQNLMSWRFIMDLVSSLSRTYKESRNAFRKALYGTTSETATWRRCAN
YVNGNMENAVGRLYVEAAFAGESKHVVEDLIAQIREVFIQTLDDLTWMDAETKK
RAEEKALAIKERIGYPDDIVSNDNKLNNEYLELNYKEDEYFENIIQNLKFSQSKQLK
KLREKVDKDEWISGAAVVNAFYSSGRNQIVFPAGILQPPFFSAQQSNSLNYGGIGM
VIGHEITHGFDDNGRNFNKDGDLVDWWTQQSASNFKEQSQCMVYQYGNFSWDL
AGGQHLNGINTLGENIADNGGLGQAYRAYQNYIKKNGEEKLLPGLDLNHKQLFFL
NFAQVWCGTYRPEYAVNSIKTDVHSPGNFRIIGTLQNSAEFSEAFHCRKNSYMNPE
KKCRVW
SEQ ID NO: 31
G399V/G714K human neprilysin variant (described by Webster et al.,
(2014), Plos One 9: e104001).
YDDGICKSSDCIKSAARLIQNMDATTEPCTDFFKYACGGWLKRNVIPETSSRYGNF
DILRDELEVVLKDVLQEPKTEDIVAVQKAKALYRSCINESAIDSRGGEPLLKLLPDI
YGWPVATENWEQKYGASWTAEKAIAQLNSKYGKKVLINLFVGTDDKNSVNHVIH
IDQPRLGLPSRDYYECTGIYKEACTAYVDFMISVARLIRQEERLPIDENQLALEMNK
VMELEKEIANATAKPEDRNDPMLLYNKMTLAQIQNNFSLEINGKPFSWLNFTNEIM
STVNISITNEEDVVVYAPEYLTKLKPILTKYSARDLQNLMSWRFIMDLVSSLSRTYK
ESRNAFRKALYVTTSETATWRRCANYVNGNMENAVGRLYVEAAFAGESKHVVED
LIAQIREVFIQTLDDLTWMDAETKKRAEEKALAIKERIGYPDDIVSNDNKLNNEYLE
LNYKEDEYFENIIQNLKFSQSKQLKKLREKVDKDEWISGAAVVNAFYSSGRNQIVF
PAGILQPPFFSAQQSNSLNYGGIGMVIGHEITHGFDDNGRNFNKDGDLVDWWTQQ
SASNFKEQSQCMVYQYGNFSWDLAGGQHLNGINTLGENIADNGGLGQAYRAYQN
YIKKNGEEKLLPGLDLNHKQLFFLNFAQVWCGTYRPEYAVNSIKTDVHSPKNFRII
GTLQNSAEFSEAFHCRKNSYMNPEKKCRVW
SEQ ID NO: 32
Native human immunoglobulin signal sequence (VH3-49)
MEFGLSWLFLVAILKGVQC
SEQ ID NO: 33
H3 Fab-linker-Neprilysin heavy chain
MEFGLSWLFLVAILKGVQCEVQLQESGGGVVQPGGSLRLSCAASGFTFSNYGMHW
IRQAPGKGLEWVSYISSGSSTIYYADSVKGRFTISRDNSKNTLYLQMNSLRSEDTAV
YYCARRGLLLDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKP
SNTKVDKKVEPKSCDKTHTGGSGGGSGGGSGGYDDGICKSSDCIKSAARLIQNMD
ATTEPCTDFFKYACGGWLKRNVIPETSSRYGNFDILRDELEVVLKDVLQEPKTEDIV
AVQKAKALYRSCINESAIDSRGGEPLLKLLPDIYGWPVATENWEQKYGASWTAEK
AIAQLNSKYGKKVLINLFVGTDDKNSVNHVIHIDQPRLGLPSRDYYECTGIYKEACT
AYVDFMISVARLIRQEERLPIDENQLALEMNKVMELEKEIANATAKPEDRNDPMLL
YNKMTLAQIQNNFSLEINGKPFSWLNFTNEIMSTVNISITNEEDVVVYAPEYLTKLK
PILTKYSARDLQNLMSWRFIMDLVSSLSRTYKESRNAFRKALYGTTSETATWRRCA
NYVNGNMENAVGRLYVEAAFAGESKHVVEDLIAQIREVFIQTLDDLTWMDAETK
KRAEEKALAIKERIGYPDDIVSNDNKLNNEYLELNYKEDEYFENIIQNLKFSQSKQL
KKLREKVDKDEWISGAAVVNAFYSSGRNQIVFPAGILQPPFFSAQQSNSLNYGGIG
MVIGHEITHGFDDNGRNFNKDGDLVDWWTQQSASNFKEQSQCMVYQYGNFSWD
LAGGQHLNGINTLGENIADNGGLGQAYRAYQNYIKKNGEEKLLPGLDLNHKQLFF
LNFAQVWCGTYRPEYAVNSIKTDVHSPGNFRIIGTLQNSAEFSEAFHCRKNSYMNP
EKKCRVW
SEQ ID NO: 34
Native human immunoglobulin signal sequence
MDMRVPAQLLGLLLLWLRGARC
SEQ ID NO: 35
L2 light chain
MDMRVPAQLLGLLLLWLRGARCDIQMTQSPSSLSASVGDRVTISCRASKSVSTSSY
SYMHWYQQKPEKAPKLLIKYASYLQSGVPSRFSGSGSGTDFTLTISSLQPEDVATY
YCQHSREFPWTFGAGTKLELKRTVAAPSVFIFPPSDEQLKSGTASVVCLLNNFYPRE
AKVQWKVDNALQSGNSQESVTEQDSKDSTYSLSSTLTLSKADYEKHKVYACEVT
HQGLSSPVTKSFNRGEC

### INCORPORATION BY REFERENCE

All publications and patents mentioned herein are hereby incorporated by reference in their entirety as if each individual publication or patent was specifically and individually indicated to be incorporated by reference.

While specific embodiments of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

We claim:

1. A chimeric polypeptide comprising: (i) a neprilysin polypeptide, and (ii) an internalizing moiety.

2. The chimeric polypeptide of claim 1, wherein the neprilysin polypeptide is a functional neprilysin polypeptide.

3. The chimeric polypeptide of claim 1 or claim 2, wherein the neprilysin polypeptide comprises a functional portion of a neprilysin polypeptide.

4. The chimeric polypeptide of any one of claims 1-3, wherein the neprilysin polypeptide comprises a functional fragment of a neprilysin polypeptide.

5. The chimeric polypeptide of any one of claims 1-4, wherein the neprilysin polypeptide does not comprise the full-length neprilysin polypeptide set forth in SEQ ID NO: 1.

6. The chimeric polypeptide of any one of claims 1-5, wherein neither the neprilysin polypeptide nor the chimeric polypeptide comprises the portion of neprilysin polypeptide set forth in residues 1-51 of SEQ ID NO: 1.

7. The chimeric polypeptide of any one of claims 1-6, wherein the neprilysin polypeptide comprises the portion of neprilysin polypeptide set forth in residues 52-750 of SEQ ID NO: 1.

8. The chimeric polypeptide of any one of claims 1-7, wherein the neprilysin polypeptide comprises the amino acid sequence of SEQ ID NO: 2.

9. The chimeric polypeptide of any one of claims 1-8, wherein the chimeric polypeptide has neprilysin activity.

10. The chimeric polypeptide of any one of claims 1-9, wherein the internalizing moiety comprises an antibody or antigen binding fragment.

11. The chimeric polypeptide of claim 10, wherein the antibody or antigen binding fragment is a chimeric, humanized, or fully human antibody or antigen binding fragment.

12. The chimeric polypeptide of any one of claims 1-11, wherein the antibody or antigen binding fragment comprises a heavy chain variable domain and a light chain variable domain.

13. The chimeric polypeptide of claim 12, wherein the heavy chain variable (VH) domain comprises the amino acid sequence of SEQ ID NO: 3.

14. The chimeric polypeptide of claim 13, wherein the VH domain comprises:
a VH CDR1 having the amino acid sequence of SEQ ID NO: 10;
a VH CDR2 having the amino acid sequence of SEQ ID NO: 11;
a VH CDR3 having the amino acid sequence of SEQ ID NO: 12,
which CDRs are according to the IMGT system.

15. The chimeric polypeptide of claim 12, wherein the light chain variable (VL) domain comprises the amino acid sequence of SEQ ID NO: 4.

16. The chimeric polypeptide of claim 15, wherein the VL domain comprises:
a VL CDR1 having the amino acid sequence of SEQ ID NO: 13;
a VL CDR2 having the amino acid sequence of SEQ ID NO: 14;
a VL CDR3 having the amino acid sequence of SEQ ID NO: 15,
which CDRs are according to the IMGT system.

17. The chimeric polypeptide of any one of claims 1-16, wherein the internalizing moiety comprises an antibody.

18. The chimeric polypeptide of claim 17, wherein the antibody is a monoclonal antibody.

19. The chimeric polypeptide of any one of claims 1-18, wherein the internalizing moiety comprises an antigen-binding fragment.

20. The chimeric polypeptide of claim 19, wherein the antigen-binding fragment is a Fab.

21. The chimeric polypeptide of claim 19, wherein the antigen-binding fragment is a Fab′.

22. The chimeric polypeptide of claim 19, wherein the antigen-binding fragment is an scFv.

23. The chimeric polypeptide of any one of claims 1-22, wherein the internalizing moiety promotes delivery of the chimeric polypeptide into cells via an equilibrative nucleoside transporter (ENT) transporter.

24. The chimeric polypeptide of any one of claims 1-23, wherein the internalizing moiety promotes delivery of the chimeric polypeptide into cells via ENT2.

25. The chimeric polypeptide of any one of claims 1-24, wherein the internalizing moiety promotes delivery of the chimeric polypeptide into a muscle cell.

26. The chimeric polypeptide of claim 25, wherein the muscle cell is a skeletal muscle cell.

27. The chimeric polypeptide of any one of claims 1-24, wherein the internalizing moiety promotes delivery of the chimeric polypeptide into a neuronal cell.

28. The chimeric polypeptide of any one of claims 1-24, wherein the internalizing moiety promotes delivery of the chimeric polypeptide into a retinal cell.

29. The chimeric polypeptide of any one of claims 1-28, wherein the neprilysin polypeptide is chemically conjugated to the internalizing moiety.

30. The chimeric polypeptide of any one of claims 1-29, wherein the chimeric polypeptide comprises a fusion protein comprising the neprilysin polypeptide and all or a portion of the internalizing moiety.

31. The chimeric polypeptide of claim 30, wherein the chimeric polypeptide does not include a linker interconnecting the neprilysin polypeptide to the internalizing moiety.

32. The chimeric polypeptide of claim 30, wherein the fusion protein comprises a linker.

33. The chimeric polypeptide of claim 32, wherein the linker conjugates or joins the neprilysin polypeptide to the internalizing moiety.

34. The chimeric polypeptide of claim 32 or 33, wherein the linker is a cleavable linker.

35. The chimeric polypeptide of any one of claims 1-28, wherein all or a portion of the internalizing moiety is conjugated or joined, directly or via a linker, to the N-terminal amino acid of the neprilysin polypeptide.

36. The chimeric polypeptide of any one of claims 1-28, wherein all or a portion of the internalizing moiety is conjugated or joined, directly or via a linker, to the C-terminal amino acid of the neprilysin polypeptide.

37. The chimeric polypeptide of any one of claims 1-28, wherein all or a portion of the internalizing moiety is conjugated or joined, directly or indirectly to an internal amino acid of the neprilysin polypeptide.

38. The chimeric polypeptide of any one of claims 1-37, wherein the chimeric polypeptide is capable of degrading protein aggregates.

39. The chimeric polypeptide of any one of claims 1-38, wherein the chimeric polypeptide is capable of degrading protein aggregates in a cell.

40. The chimeric polypeptide of any one of claims 1-39, wherein the chimeric polypeptide is capable of degrading protein aggregates in a cell from a subject having Age-related Macular Degeneration (AMD), Inclusion Body Myositis (IBM), Myofibrillar Myopathy 2 (MFM2), Nemaline Myopathy (NM), or Alzheimer's Disease.

41. The chimeric polypeptide of claim 40, wherein the subject is a non-human animal.

42. The chimeric polypeptide of claim 41, wherein the non-human animal is a mouse.

43. The chimeric polypeptide of claim 40, wherein the subject is a human.

44. The chimeric polypeptide of any one of claims 40-43, wherein the cell is in vitro.

45. The chimeric polypeptide of any one of claims 40-43, wherein the cell is a muscle cell.

46. The chimeric polypeptide of any one of claims 40-43, wherein the cell is a skeletal muscle cell.

47. The chimeric polypeptide of any one of claims 40-43, wherein the cell is a neuronal cell.

48. The chimeric polypeptide of any one of claims 40-43, wherein the cell is a retinal cell.

49. The chimeric polypeptide of any one of claims 1-48, wherein the chimeric polypeptide is produced recombinantly.

50. The chimeric polypeptide of claim 49, wherein the chimeric polypeptide is produced in a prokaryotic or eukaryotic cell.

51. The chimeric polypeptide of claim 50, wherein the eukaryotic cell is selected from a yeast cell, an avian cell, an insect cell, or a mammalian cell.

52. A nucleic acid construct, comprising a nucleotide sequence that encodes the chimeric polypeptide of any one of claims 1-51 as a chimeric polypeptide comprising a fusion protein.

53. The nucleic acid construct of claim 52, wherein the nucleotide sequence is codon optimized for expression in a mammalian cell.

54. The nucleic acid construct of claim 53, wherein the mammalian cell is a CHO cell or a HEK-293 cell.

55. A set of nucleic acid constructs, together comprising nucleotide sequences that encode the chimeric polypeptide of any one of claims 1-51.

56. The set of nucleic acid constructs of claim 55, wherein the nucleotide sequences are codon optimized for expression in a mammalian cell.

57. The set of nucleic acid constructs of claim 56, wherein the mammalian cell is a CHO cell or a HEK-293 cell.

58. A vector comprising the nucleic acid construct of any one of claims 52-54.

59. A set of vectors comprising the set of nucleic acid constructs of any one of claims 55-57.

60. A host cell comprising the vector of claim 58 or 59.

61. A method for delivering neprilysin activity into a cell from or of a subject having Age-related Macular Degeneration (AMD), comprising contacting the cell with the chimeric polypeptide of any one of claims 1-51.

62. A method for delivering neprilysin activity into a cell from or of a subject having Inclusion Body Myositis (IBM), comprising contacting the cell with the chimeric polypeptide of any one of claims 1-51.

63. A method for delivering neprilysin activity into a cell from or of a subject having Myofibrillar Myopathy 2 (MFM2), comprising contacting the cell with the chimeric polypeptide of any one of claims 1-51.

64. A method for delivering neprilysin activity into a cell from or of a subject having Nemaline Myopathy (NM), comprising contacting the cell with the chimeric polypeptide of any one of claims 1-51.

65. The method of any one of claims 61-64, wherein the subject is a non-human animal.

66. The method of claim 65, wherein the non-human animal is a mouse.

67. The method of any one of claims 61-64, wherein the subject is a human.

68. The method of any one of claims 61-67, wherein the cell is in the subject.

69. The method of any one of claims 61-68, wherein the cell is a muscle cell.

70. The method of any one of claims 61-68, wherein the cell is a skeletal muscle cell.

71. The method of any one of claims 61-68, wherein the cell is a brain cell.

72. The method of any one of claims 61-68, wherein the cell is a retinal cell.

73. The method of any one of claims 61-64, wherein the cell is in vitro.

74. A method for treating a subject having Age-related Macular Degeneration (AMD), comprising administering to the subject a therapeutically effective amount of the chimeric polypeptide of any one of claims 1-51.

75. A method for treating a subject having Inclusion Body Myositis (IBM), comprising administering to the subject a therapeutically effective amount of the chimeric polypeptide of any one of claims 1-51.

76. A method for treating a subject having Myofibrillar Myopathy 2 (MFM2), comprising administering to the subject a therapeutically effective amount of the chimeric polypeptide of any one of claims 1-51.

77. A method for treating a subject having Nemaline Myopathy (NM), comprising administering to the subject a therapeutically effective amount of the chimeric polypeptide of any one of claims 1-51.

78. The method of any one of claims 74-77, wherein the subject is a non-human animal.

79. The method of claim 78, wherein the subject is a mouse.

80. The method of any one of claims 74-77, wherein the subject is a human.
